(19) United States
(12) Patent Application Publication
(10) Pub. No.: US 2024/0287176 A1
(43) Pub. Date: Aug. 29, 2024

(54) METHODS OF TREATMENT OF AUTOIMMUNE DISORDERS USING ILT7 BINDING PROTEINS

(71) Applicant: Viela Bio, Inc., Deerfield, IL (US)

(72) Inventors: Jorn DRAPPA, La Jolla, CA (US); John Nolan RATCHFORD, Lutherville, MD (US); Edward R. HAMMOND, Baltimore, MD (US); William A. REES, North Bethesda, MD (US); Gabor G. ILLEI, Rockville, MD (US); Li YAN, Palo Alto, CA (US)

(21) Appl. No.: 18/558,505

(22) PCT Filed: May 4, 2022
(86) PCT No.: PCT/US2022/027620

§ 371 Date: Nov. 1, 2023

Related U.S. Application Data
(60) Provisional application No. 63/183,886, filed on May 4, 2021.
(60) Provisional application No. 63/197,789, filed on Jun. 7, 2021.
(60) Provisional application No. 63/242,768, filed on Sep. 10, 2021.
(60) Provisional application No. 63/249,953, filed on Sep. 29, 2021.
(60) Provisional application No. 63/326,424, filed on Apr. 1, 2022.

(51) Int. Cl. C07K 16/28; A61K 31/573; A61K 39/00; A61P 17/14; A61P 37/02

(52) U.S. Cl. CPC C07K 16/2803; A61K 31/573; A61P 17/14; A61P 37/02; A61K 2039/505; A61K 2039/545

(57) ABSTRACT

The present disclosure is related to methods of treating an autoimmune disorder in a subject in need thereof comprising administering an immunoglobulin-like transcript 7 (ILT7) binding protein. The present disclosure also relates to methods of reducing plasmacytoid dendritic cells (pDCs), or reducing a type I interferon gene signature (IFNGS) in a tissue, of a subject in need thereof by administering an ILT7-binding protein to the subject.

### CROSS-REFERENCE TO RELATED APPLICATIONS

This application claims priority to U.S. Provisional Patent Application No. 63/183,886 filed May 4, 2021, U.S. Provisional Patent Application No. 63/197,789 filed Jun. 7, 2021, U.S. Provisional Patent Application No. 63/242,768 filed Sep. 10, 2021, U.S. Provisional Patent Application No. 63/249,953 filed Sep. 29, 2021, and U.S. Provisional Patent Application No. 63/326,424 filed Apr. 1, 2022, each of which is incorporated by reference herein in their entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing filename: HOPA_033_05 WO_SeqList_ST25.txt, date recorded: May 2, 2022, file size 12,288 bytes.

### TECHNICAL FIELD

The present disclosure is related to methods of treating an autoimmune disorder in a subject in need thereof comprising administering an immunoglobulin-like transcript 7 (ILT7) binding protein.

### BACKGROUND

The type I interferon (IFN) axis is one of the most significant pathways in human disease, and its dysregulation is central to the pathogenesis of many chronic autoimmune diseases, such as systemic lupus erythematosus (SLE). Although the precise etiology of SLE and other autoimmune diseases is not fully resolved, it is believed that a combination of environmental and genetic factors, together with an accumulation of cellular debris, leads to a breakdown in peripheral immune tolerance, characterized by high levels of circulating autoreactive antibodies. Currently available methods are directed towards treating autoimmune diseases and not towards preventing such diseases. Further, conventional treatment options for autoimmune diseases include immunosuppressant drugs that are associated with a wide range of side effects. Thus, there is a need for better therapeutic alternatives for treating and preventing autoimmune diseases. The present disclosure addresses these needs.

### SUMMARY

Provided are methods of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is from about 100-350 mg. In aspects, the pharmaceutically effective amount is administered in one or more administrations (e.g., 1, 2, or 3) in the same day. In aspects, the autoimmune disease is selected from the group consisting of discoid lupus erythematosus (DLE), systemic lupus erythematosus (SLE), lupus nephritis, Dermatomyositis, Anti-Synthetase Inflammatory Myositis, and alopecia areata. In aspects, the autoimmune disease is SLE, and wherein the pharmaceutically effective amount is about 200 mg. In aspects, the administering is effective in reducing: a) a level of plasmacytoid dendritic cells (pDCs) in a tissue of the subject; b) a type I interferon gene signature (IFNGS); or c) the level of plasmacytoid dendritic cells (pDCs) in the tissue of the subject and the type I IFNGS, each of each is as compared to a baseline level of the subject before the administering. In aspects, the IFNGS comprises the collective expression levels of SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18. In aspects, the reduction in the level of pDCs in the tissue compared to the baseline value is at least about 10%, 20%, 25%, 30%, 40%, 50%, 60%, or 70%. In aspects, the autoimmune disease is lupus nephritis, and wherein the pharmaceutically effective amount is about 300 mg. In aspects, wherein after the administering, the subject achieves a positive renal response as determined by one or more of an improvement in a Glomerular Filtration Rate (eGFR) or 24-hour Urine Protein to Creatinine Ratio (UPCR) as compared to a baseline level of the subject before the administering. In aspects, the autoimmune disease is discoid lupus erythematosus (DLE), and wherein the pharmaceutically effective amount is about 150-300 mg. In aspects, the administering is effective in reducing one or more of: Cutaneous Lupus Erythematosus Disease Area and Severity Index-Activity (CLASI-A) score, Cutaneous Lupus Erythematosus Disease Area and Severity Index-Damage (CLASI-D) score, Cutaneous Lupus Activity Investigator's Global Assessment (CLA-IGA) scale, Discoid Lupus Erythematosus Classification Criteria (DLECC) score, Score of Activity and Damage in Discoid Lupus Erythematosus (SADDLE), onset of new discoid lesions, size of lesions, or dyspigmentation of a discoid lesion, as compared to a baseline level of the subject before the administering. In aspects, the autoimmune disease is alopecia areata, and wherein the pharmaceutically effective amount is about 300 mg. In aspects, the administering is effective in stabilizing or reducing hair loss in the subject as determined by a stable or reduced Severity of Alopecia Tool (SALT) score and/or Alopecia Density and Extent (ALODEX) score. In aspects, the ILT7-binding protein is administered with one or more additional therapies. In aspects, the one or more additional therapies comprises a corticosteroid. In aspects, the corticosteroid is prednisone. In aspects, the administration of the one or more additional therapies is tapered. In aspects, the pharmaceutically effective amount of the ILT7-binding protein is from about 150-300 mg. In aspects, the pharmaceutically effective amount of the ILT7-binding protein is from about 200-300 mg. In aspects, the ILT7-binding protein is administered once about every four weeks or once about every twelve weeks. In aspects, the pharmaceutically effective amount of the ILT7-binding protein is about 300 mg and the 300 mg is administered as two doses of 150 mg each.

Provided are methods of treating discoid lupus erythematosus (DLE) in a subject in need thereof, the method comprising administering to the subject a pharmaceutically effective amount of an immunoglobulin-like transcript 7 (ILT7)-binding protein, wherein the pharmaceutically effective amount of the ILT7-binding protein is from about 100 to about 300 mg.

Provided are methods of treating systemic lupus erythematosus (SLE) in a subject in need thereof, the method comprising administering to the subject a pharmaceutically effective amount of an immunoglobulin-like transcript 7 (ILT7)-binding protein, wherein the pharmaceutically effective amount of the ILT7-binding protein is from about 200 mg.

Provided are methods of treating lupus nephritis in a subject in need thereof, the method comprising administering to the subject a pharmaceutically effective amount of an immunoglobulin-like transcript 7 (ILT7)-binding protein, wherein the pharmaceutically effective amount of the ILT7-binding protein is from about 300 mg.

Provided are methods of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is from about 300 mg.

Provided are methods of treating dermatomyositis in a subject in need thereof, the method comprising administering to the subject a pharmaceutically effective amount of an immunoglobulin-like transcript 7 (ILT7)-binding protein.

Provided are methods of treating Anti-Synthetase Inflammatory Myositis in a subject in need thereof, the method comprising administering to the subject a pharmaceutically effective amount of an immunoglobulin-like transcript 7 (ILT7)-binding protein.

In aspects, the pharmaceutically effective amount of the ILT7-binding protein of any of the provided methods of treatment is from about 100 mg to about 300 mg. In aspects, ILT7 binding protein is an antibody that comprises heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOs: 3, 4, 5, 6, 7, and 8, respectively. In aspects, ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2. In aspects, the ILT7-binding protein is an antibody comprising a heavy chain variable region (VH) of SEQ ID NO: 1 and a light chain variable region (VL) of SEQ ID NO:2. In aspects, the ILT7 binding protein is afucosylated. In aspects, the ILT7 binding protein is Daxdilimab. In aspects, the administration is subcutaneous. In aspects, the subject is administered the ILT7-binding protein every 4 weeks. In aspects, the subject is administered the ILT7-binding protein every 12 weeks. In aspects, prior to the administering, the subject is administered at least one initial dose of the ILT7-binding protein. In aspects, the at least one initial dose is administered every 2 weeks for 1, 2, 3, 4, 5, or more doses. In aspects, the at least one initial dose is about 100-300 mg.

Provided are methods of treating lupus nephritis in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 100 mg every 2 weeks for up to 4 weeks followed by 100 mg every 4 weeks for 1, 2, 3, 4, 5, 6, 7, 8, 9, 10 11, 12, 13, 14, 15 or more doses. In aspects, the subject is further administered: 100 mg of the ILT7 binding protein every 12 weeks; or 300 mg of the ILT7 binding protein every 12 weeks following the every 4 weeks administration. In aspects, the 100 mg every 12 weeks or the 300 mg every 12 weeks are continued for at least about 104 weeks.

Provided are methods of treating lupus nephritis in a subject in need thereof, the method comprising administering to the subject a pharmaceutically effective amount of immunoglobulin-like transcript 7 (ILT7)-binding protein, wherein the pharmaceutically effective amount of the ILT7-binding protein is about 300 mg every 2 weeks for up to 4 weeks followed by 300 mg every 12 weeks for 1, 2, 3, 4, 5, 6, 7, 8, 9, 10 11, 12, 13, 14, 15 or more doses.

The methods of the present disclosure can be used for treating autoimmune disorders in a subject in need thereof, reducing pDCs in a tissue of a subject in need thereof, and for reducing a type I IFNGS in a subject in need thereof. The present disclosure also relates to methods of reducing plasmacytoid dendritic cells (pDCs), or reducing a type I interferon gene signature (IFNGS) in a tissue, of a subject in need thereof by administering an ILT7-binding protein to the subject.

The disclosure provides methods of treating an autoimmune disorder in a subject in need thereof. In the method, a pharmaceutically effective amount of an immunoglobulin-like transcript 7 (ILT7)-binding protein is administered to the subject. In aspects, the effective amount of the ILT7-binding protein is about 200 mg. In aspects, the subject is administered: (a) a first dose, (b) a second dose about four weeks after the first dose, (c) a third dose about twelve weeks after the first dose, and (d) subsequent doses about twelve weeks after the third dose. In aspects, the ILT7-binding protein is administered once about every four weeks.

The disclosure also provides methods of reducing pDCs in a tissue of a subject in need thereof by administering an effective amount of an ILT7-binding protein to a subject. In aspects, the effective amount of the ILT7-binding protein is about 200 mg.

The disclosure also provides methods for reducing a type I IFNGS in a subject in need thereof. In the method, a pharmaceutically effective amount of an ILT7-binding protein is administered to the subject in need thereof when the subject in need thereof's type I IFNGS is elevated relative to a normal subject's type I IFNGS. The pharmaceutically effective amount of the ILT7-binding protein is about 200 mg.

In aspects, the disclosure provides a method of treating an autoimmune disorder in a subject in need thereof. In the method, a pharmaceutically effective amount of an ILT7-binding protein is administered to the subject. The pharmaceutically effective amount of the ILT7-binding protein is about 200 mg. The ILT7-binding protein is administered once about every twelve weeks. Additionally, the subject is administered a 200 mg dose of the ILT7-binding protein about four weeks after an initial dose.

In aspects, the disclosure provides a method of reducing pDCs in a tissue of a subject in need thereof. In the method, a pharmaceutically effective amount of an ILT7-binding protein is administered to the subject. The pharmaceutically effective amount of the ILT7-binding protein is about 200 mg. The subject is administered: (a) a first dose, (b) a second dose about four weeks after the first dose, (c) a third dose about twelve weeks after the first dose, and (d) subsequent doses about twelve weeks after the third dose.

In aspects, the disclosure provides a method of reducing pDCs in a tissue of a subject in need thereof. In the method, a pharmaceutically effective amount of an ILT7-binding protein is administered to the subject. The pharmaceutically effective amount of the ILT7-binding protein is about 200 mg. The ILT7-binding protein is administered once about every four weeks.

In aspects, the disclosure provides a method of reducing pDCs in a tissue of a subject in need thereof. In the method, a pharmaceutically effective amount of an ILT7-binding protein is administered to the subject. The pharmaceutically effective amount of the ILT7-binding protein is about 200 mg. The ILT7-binding protein is administered once about every twelve weeks. Additionally, the subject is administered a 200 mg dose of the ILT7-binding protein about four weeks after an initial dose.

In aspects, the disclosure provides a method of treating an autoimmune disorder in a subject in need thereof. In the method, a pharmaceutically effective amount of an ILT7-binding protein is administered to the subject. The pharmaceutically effective amount of the ILT7-binding protein is about 200 mg. The ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2. The subject is administered: (a) a first dose, (b) a second dose about four weeks after the first dose, (c) a third dose about twelve weeks after the first dose, and (d) subsequent doses about twelve weeks after the third dose.

In aspects, the disclosure provides a method of treating systemic lupus erythematosus (SLE) in a subject in need thereof. In the method, a pharmaceutically effective amount of an ILT7-binding protein is administered to the subject. The pharmaceutically effective amount of the ILT7-binding protein is about 200 mg. The ILT7 binding protein is an antibody comprising a VH that is at least 85%, 90%, 95%, 96%, 97%, 98% or 99% identical to SEQ ID NO:1 and/or a VL that is at least 85%, 90%, 95%, 96%, 97%, 98% or 99% identical to SEQ ID NO:2. The subject is administered: (a) a first dose, (b) a second dose about four weeks after the first dose, (c) a third dose about twelve weeks after the first dose, and (d) subsequent doses about twelve weeks after the third dose.

In aspects, the disclosure provides a method of treating an autoimmune disorder in a subject in need thereof. In the method, a pharmaceutically effective amount of an ILT7-binding protein is administered to the subject. The pharmaceutically effective amount of the ILT7-binding protein is about 200 mg. The ILT7 binding protein is an antibody comprising a VH that is at least 85%, 90%, 95%, 96%, 97%, 98% or 99% identical to SEQ ID NO: 1 and/or a VL that is at least 85%, 90%, 95%, 96%, 97%, 98% or 99% identical to SEQ ID NO:2. The ILT7-binding protein is administered once about every four weeks.

In aspects, the disclosure provides for a method of treating SLE in a subject in need thereof. In the method, a pharmaceutically effective amount of an ILT7-binding protein is administered to the subject. The pharmaceutically effective amount of the ILT7-binding protein is about 200 mg. The ILT7 binding protein is an antibody comprising a VH that is at least 85%, 90%, 95%, 96%, 97%, 98% or 99% identical to SEQ ID NO: 1 and/or a VL that is at least 85%, 90%, 95%, 96%, 97%, 98% or 99% identical to SEQ ID NO:2. The ILT7-binding protein is administered once about every four weeks.

In aspects, the disclosure provides a method of treating an autoimmune disorder in a subject in need thereof. In the method, a pharmaceutically effective amount of an ILT7-binding protein is administered to the subject. The pharmaceutically effective amount of the ILT7-binding protein is about 200 mg. The ILT7 binding protein is an antibody comprising a VH that is at least 85%, 90%, 95%, 96%, 97%, 98% or 99% identical to SEQ ID NO: 1 and/or a VL that is at least 85%, 90%, 95%, 96%, 97%, 98% or 99% identical to SEQ ID NO:2. The ILT7-binding protein is administered once about every twelve weeks. Additionally, the subject is administered a 200 mg dose of the ILT7-binding protein about four weeks after an initial dose.

In aspects, the disclosure provides a method of treating SLE in a subject in need thereof. In the method, a pharmaceutically effective amount of an ILT7-binding protein is administered to the subject. The pharmaceutically effective amount of the ILT7-binding protein is about 200 mg. The ILT7 binding protein is an antibody comprising a VH that is at least 85%, 90%, 95%, 96%, 97%, 98% or 99% identical to SEQ ID NO:1 and/or a VL that is at least 85%, 90%, 95%, 96%, 97%, 98% or 99% identical to SEQ ID NO:2. The ILT7-binding protein is administered once about every twelve weeks. Additionally, the subject is administered a 200 mg dose of the ILT7-binding protein about four weeks after an initial dose.

In aspects, the disclosure provides a method of treating an autoimmune disorder in a subject in need thereof. In the method, a pharmaceutically effective amount of an ILT7-binding protein is administered to the subject. The pharmaceutically effective amount of the ILT7-binding protein is about 200 mg. The ILT7 binding protein is an antibody comprising a VH that is at least 85%, 90%, 95%, 96%, 97%, 98% or 99% identical to SEQ ID NO: 1 and/or a VL that is at least 85%, 90%, 95%, 96%, 97%, 98% or 99% identical to SEQ ID NO:2. These and other embodiments are described below.

Provided is a method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 250 mg to 350 mg.

Provided is a method of reducing plasmacytoid dendritic cells (pDCs) in a tissue of a subject with alopecia areata, the method comprising administering to the subject a pharmaceutically effective amount of an immunoglobulin-like transcript 7 (ILT7)-binding protein, wherein the pharmaceutically effective amount of the ILT7-binding protein is about 250 mg to 350 mg.

In aspects, the administering of the ILT7-binding protein reduces plasmacytoid dendritic cells (pDCs) in the subject. In aspects, the pDCs are circulating pDCs. In aspects, the reduction in the pDCs is reversible. In aspects, the ILT7-binding protein is administered once about every four weeks.

Provided is a method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 250 mg to 350 mg, and wherein the ILT7-binding protein is administered once about every four weeks.

Provided is a method of reducing plasmacytoid dendritic cells (pDCs) in a tissue of a subject with alopecia areata, the method comprising administering to the subject a pharmaceutically effective amount of an immunoglobulin-like transcript 7 (ILT7)-binding protein, wherein the pharmaceutically effective amount of the ILT7-binding protein is about 250 mg to 350 mg, and wherein the ILT7-binding protein is administered once about every four weeks.

In aspects, the reduction in pDCs in the tissue compared to the baseline value ranges from about 1% to about 99%. In aspects, the decrease in pDCs in the tissue compared to the baseline value is at least about 50%. In aspects, the ILT7-binding protein induces antibody-dependent cell-mediated cytotoxicity (ADCC) activity against pDCs. In aspects, the ILT7-binding protein at least suppresses release of type I interferon (IFN) from pDCs. In aspects, the type I IFN is IFNα. In aspects, the ILT7-binding protein binds to ILT7. In aspects, the ILT7-binding protein is an antibody comprising heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOS: 3, 4, 5, 6, 7, and 8, respectively. In aspects, the ILT7 binding protein is an antibody comprising a variable heavy chain (VH) that is at least 85%, 90%, 95%, 96%, 97%, 98% or 99% identical to SEQ ID NO: 1 and/or a variable light chain (VL) that is at least 85%, 90%, 95%, 96%, 97%, 98% or 99% identical to SEQ ID NO:2. In aspects, the ILT7-binding protein is an antibody comprising a heavy chain variable region (VH) of SEQ ID NO:1 and a light chain variable region (VL) of SEQ ID NO:2. In aspects, the ILT7 binding protein is afucosylated.

Provided is a method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 250 mg to 350 mg, wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2, and wherein the ILT7-binding protein is administered once about every four weeks.

Provided is a method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 250 mg-350 mg, and wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2.

In aspects, the ILT7-binding protein is administered with one or more additional therapies. In aspects, one of the one or more additional therapies is a standard of care therapy. In aspects, the ILT7-binding protein is administered subcutaneously. In aspects, the administering is effective in stabilizing or reducing hair loss in the subject as determined by a stable or reduced Severity of Alopecia Tool (SALT) score and/or Alopecia Density and Extent (ALODEX) score. In aspects, the ILT7-binding protein is administered to a mouse with alopecia acreata, the administering is effective in reducing a level of type 1 interferon-inducible myxovirus protein A (MxA) in a biopsy comprising a hair follicle of the mouse as determined by immunohistochemistry. In aspects, the pharmaceutically effective amount of the ILT7-binding protein is about 300 mg. In aspects, the 300 mg amount is administered as two separate doses of 150 mg each that are administered on the same day. In aspects, the ILT7 binding protein is Daxdilimab.

### BRIEF DESCRIPTION OF THE FIGURES

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 shows the overall study design. COVID-19: coronavirus disease 2019; OGC: oral glucocorticoid; Q4W: every 4 weeks; Q12W: every 12 weeks; SC: subcutaneous(ly); SLE: systemic lupus erythematosus; SLEDAI-2K: SLE Disease Activity Index 2000; W: week.

FIG. 2. Shows a clinical study diagram of Example 2.

FIG. 3 shows an exemplary oral glucocorticoid tapering schedule.

FIG. 4 depicts an exemplary study diagram of Example 3.

FIG. 5 shows the Severity of Alopecia Tool (SALT) assessment tool from Olsen E A et al., Alopecia areata investigational assessment guidelines—Part II. National Alopecia Areata Foundation. J Am Acad Dermatol. 2004; 51(3):440-447.

FIG. 6 shows the Alopecia Density and Extent (ALODEX) assessment tool from Olsen E A et al., SALT II: A new take on the Severity of Alopecia Tool (SALT) for determining percentage scalp hair loss. J Am Acad Dermatol. 2016; 75(6): 1268-1270 and Olsen E A et al., Objective outcome measures: Collecting meaningful data on alopecia areata. J Am Acad Dermatol. 2018; 79(3):470-478 e473.

FIG. 7 shows the overall study design of Example 4. CRR: complete renal response; D: day; IP: investigational product; MMF: mycophenolate mofetil; MPA: mycophenolic acid; OCS: oral corticosteroids; PRR: partial renal response; Q4W: once every 4 weeks; Q12W: once every 12 weeks; SC: subcutaneously; SFU: Safety Follow-Up; SOC: standard-of-care; W: week.

FIG. 8 shows the overall study design of Example 5. LTE: long-term extension; N: total population; Q4W: once every 4 weeks; SC: subcutaneously; SFU: Safety Follow-up; W: week.

### DETAILED DESCRIPTION

The disclosure provides methods for of treating an autoimmune disorder in a subject with an ILT7-binding protein. In particular aspects, the ILT-7 binding protein is administered at a dose of 200 mg. In aspects, the ILT7-binding protein is administered once about every four weeks. In other aspects, the ILT-7 binding protein is administered once about every twelve weeks. In aspects, the methods provide treating an autoimmune disorder in a subject in need thereof, wherein the subject is determined to have a high blood type I interferon gene signature (IFNGS) level. The disclosure also provides methods for reducing the IFNGS in a subject in need thereof. In aspects, the ILT7 binding protein is administered to the subject when the type I IFNGS is elevated in the subject relative to the type I IFNGS in a normal subject. In aspects, for example, the ILT7 binding protein is administered to subjects with elevated baseline type I IFNGS relative to the type I IFNGS in a normal subject. In particular aspects, the ILT7-binding protein is an antibody. In aspects, the antibody is daxdilimab.

In certain diseases (e.g., autoimmune diseases), activated pDCs secrete significant amounts of type I and type III interferons (IFNs). Type I IFNs are a large group of IFN proteins that help regulate the immune system. The mammalian IFNs are designated IFNα, IFNβ, IFN®, IFNε, IFNκ, IFNτ, IFNδ, IFNζ, and IFNν. In aspects, the type I IFN that generates the type I IFNGS is IFNα. Type I IFN protein levels cannot be directly measured in a reliable way; however, measurement of IFN-inducible genes serves as a robust surrogate to Type 1 IFN protein levels. The expression levels of these type I IFN-inducible genes can be measured in biological samples (e.g., blood, skin, skeletal muscles, etc.) and analyzed as a composite outcome referred to as the “type I interferon gene signature” or “type I IFNGS” or “IFNGS.”

### Definitions

Unless defined otherwise, all technical and scientific terms used herein have the same meaning as commonly understood by one of ordinary skill in the art to which the subject matter pertains. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

As used in this specification and the appended claims, the singular forms “a,” “an” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5. The term “about” also accounts for typical error or imprecision in measurement of values.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

By “test biological sample” is intended any biological sample obtained from an individual affected, likely to be affected, or suspected to be affected with a disease or condition such as an autoimmune disorder and/or from an individual exhibiting one or more symptoms thereof, such as but not limited to elevated type I IFNGS.

By “normal biological sample” is intended any biological sample obtained from a normal subject.

As used herein, the term “subject” refers to any individual, e.g., a human or a non-human mammal, for whom diagnosis, prognosis, or therapy is desired. The term “subject” may mean a human or non-human mammal affected, likely to be affected, or suspected to be affected with a disease, e.g., an autoimmune disease or condition. The terms “subject” and “patient” are used interchangeably herein. Although the ILT7-binding protein compositions provided herein are principally directed to compositions which are suitable for administration to humans, the skilled artisan will understand that such compositions are generally suitable for administration to subjects of all sorts. In aspects, the subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile, fish, or the like.

As used herein, the term “a subject in need thereof” includes subjects that could or would benefit from the methods described herein. Subjects in need of treatment include, without limitation, those already with the condition or disorder, those prone to having the condition or disorder, those in which the condition or disorder is suspected, as well as those in which the condition or disorder is to be prevented, ameliorated, or reversed.

As used herein, the term “normal subject” refers to any healthy individual, e.g., a human or a non-human mammal, not affected with any disease or suspected of being affected with a disease or condition. The term “normal subject” also refers to an individual e.g., a human or a non-human mammal, prior to exhibiting any symptoms associated with an autoimmune disorder, such as elevated type I IFNGS. The normal subject can be the same subject as the subject in need of treatment, prior to the subject exhibiting any symptoms of an autoimmune disorder, such as but not limited to elevated type I IFNGS. In other aspects, the normal subject and the subject in need of treatment are two different individuals.

As used herein, “treating” or “treat” describes the management and care of a subject for the purpose of combating a disease, condition, or disorder and includes the administration of an ILT7-binding protein used in the methods described herein to alleviate the symptoms or complications of a disease, condition or disorder, or to eliminate the disease, condition or disorder. Thus, the term “treat” or “treating” refers to both therapeutic measures and prophylactic or preventative measures, wherein the objective is to prevent, slow down (lessen), or ameliorate the progression of a disease (e.g., an autoimmune disease). Beneficial or desired clinical results include, but are not limited to, alleviation of symptoms, diminishing the extent of the disease, stabilized (i.e., not worsening) state of the disease, delaying or slowing of disease progression, amelioration or palliation of the disease state, and reversing the disease (whether partial or total). The term “treat” can also include treatment of a cell in vitro or an animal model.

In aspects, treatment includes the application or administration of the ILT7-binding protein used in the methods described herein to a subject in need thereof or to a subject that is suspected of needing treatment thereof, or application or administration of the ILT7-binding protein used in the methods described herein to an isolated tissue or cell line from a subject, where the subject has a disease, a symptom of a disease, or a predisposition toward a disease (e.g., an autoimmune disease). A subject may be suspected of needing the treatments described herein when the subject is exhibiting symptoms of a condition or disease by excess pDC numbers or activity, even though a formal diagnosis, has not been ascertained. In aspects, the subject suspected of needing treating has a high baseline blood type I IFNGS level. In other aspects, treatment is also intended to include the application or administration of a pharmaceutical composition comprising a ILT7-binding protein used in the methods described herein to a subject in need thereof or to a subject that is suspected of needing treatment thereof, or application, or administration of a pharmaceutical composition comprising a ILT7-binding protein used in the methods described herein to an isolated tissue or cell line from a subject who has a disease, a symptom of a disease, or a predisposition toward a disease (e.g., an autoimmune disease).

The term “reduce,” “reducing,” or “reduction” means to diminish in extent, level, amount, activity, or degree compared to an initial value. The reduction need not be statistically significant from one value over the next.

As used herein, the term “reduction in pDCs” or “reducing pDCs” refers to diminished levels of activated pDCs in a subject or in a biological sample (e.g., blood and/or other tissues such as skin cells, skin biopsy samples, etc.) taken from the subject, diminished levels of the total number of pDCs in a subject or in a biological sample taken from the subject, or both.

As used herein, the term “high” or “elevated” when used in conjunction with IFNGS means that the type I IFNGS is a fold change of at least about 1.1 to about 1000 compared to normal type I IFNGS. By “normal type I IFNGS” is intended a type I IFNGS obtained from a normal subject. The terms “high” or “elevated” when used in conjunction with type I IFNGS are used interchangeably. In aspects, the type I IFNGS is “high” or “elevated” when the type I IFNGS used in the methods described herein is at least about 1.1, 1.2, 1.3, 1.4, 1.5, 2, 3, 4, 5, 6, 7, 8, 9, 10, 15, 20, 30, 40, 50, 60, 70, 80, 90, or 100-fold relative to the type I IFNGS in a normal subject. In aspects, the methods of treatment described herein are applied when type I IFNGS is elevated by at least about 4-fold relative to normal type I IFNGS.

The terms “administer,” “administration,” “administering” and the like, as they apply to, for example, a subject, cell, tissue, organ, or biological sample, refer to contact of a compound or reagent to the subject, cell, tissue, organ, or biological sample. In the context of a cell, administration includes contact (e.g., in vitro or ex vivo) of a reagent to the cell, as well as contact of a reagent to a fluid, where the fluid is in contact with the cell. The ILT7-binding proteins used in the method described herein may be administered to a subject via a variety of routes known in the art. Exemplary routes of administering of the ILT7-binding proteins used in the methods described herein include, but are not limited to, parenteral, oral, mucosal, topical, transdermal, inhalation, sublingual, buccal, rectal, vaginal, and intranasal. The term parenteral, as used herein, includes subcutaneous injections, intravenous, intramuscular, intrasternal injection or infusion techniques. In aspects, the ILT7-binding proteins used in the methods described herein are administered intravenously. In aspects, the ILT7-binding proteins used in the methods described herein are administered by subcutaneous injection. The term “administer,” “administration,” or “administering” may involve a single administration or multiple administrations of an ILT7-binding protein used in the methods described herein. For example, multiple administration involves at least two (i.e., 2, 3, 4, 5, 6, 7, 8, 9, 10, or more) administrations to a subject of an ILT7-binding protein used in the methods described herein.

A “therapeutically effective amount,” or “pharmaceutically effective amount,” or “effective amount” of a compound (e.g., an ILT7-binding protein used in the methods described herein) refers to an amount that is sufficient to produce a desired prophylactic, therapeutic or ameliorative response in a subject, or an amount that is sufficient to result in prevention or amelioration of one or more symptoms of a disease or condition in a statistically significant manner. When referring to an individual active ingredient administered alone, a therapeutically effective dose refers to that ingredient alone. When referring to a combination, a therapeutically effective dose refers to combined amounts of the active ingredients that result in the therapeutic effect, whether administered serially or simultaneously. As used herein, the term “therapeutically effective amount” means that the ILT7-binding proteins used in the methods described herein are able to exert a medically beneficial effect (e.g., cause a reduction in an elevated type I IFNGS and/or reduction in pDCs in a subject in need thereof) when used as prescribed or directed, as compared to a placebo. The therapeutically effective amount will vary depending upon the species and weight of the subject to be administered, but may be ascertained using standard techniques.

### Immunoglobulin-Like Transcript 7 (ILT7)-Binding Protein

Generally, the terms “ILT7-binding protein,” “ILT7-binding molecule,” and “ILT7-binding protein used in the methods described herein” are used interchangeably to refer to a protein or molecule that specifically binds to immunoglobulin-like transcript 7 (ILT7). The terms protein and peptide can be used interchangeably herein. In aspects, the ILT7-binding proteins used in the methods described herein bind to full-length ILT7. In aspects, the ILT7-binding proteins used in the methods described herein bind to a fragment of ILT7. In aspects, the fragment of ILT7 to which the ILT7 binding proteins bind comprises the extracellular domain of ILT7.

In aspects, the ILT7-binding proteins used in the methods disclosed herein bind to any mammalian ILT7. In aspects, the ILT7-binding proteins used in the methods disclosed herein bind to human ILT7 or a fragment thereof, for example the extracellular portion of human ILT7. In other aspects, the ILT7-binding proteins used in the methods disclosed herein bind to cynomolgus ILT7 or a fragment thereof, for example the extracellular portion of cynomolgus ILT7. In aspects, the ILT7 binding protein binds to the Ig1 region of ILT7. In aspects, the ILT7 binding protein binds to the Ig2 region of ILT7. In aspects, the ILT7 binding protein binds to human and cynomolgus ILT7.

In aspects, the ILT7 binding protein comprises an antibody or antigen-binding fragment thereof. In aspects, the antibody or antigen-binding fragment thereof is afucosylated.

ILT7 is a cell-surface protein that is unique to plasmacytoid dendritic cells (pDCs) in human and the nonhuman primates. Provided are ILT7 binding proteins, compositions comprising the same, and methods of using the same. In aspects, a composition may comprise an effective amount of an anti-ILT7 binding protein.

Examples of ILT7-binding proteins are disclosed and described in PCT Publication No. WO 2017/156298, U.S. Pat. No. 8,084,585B2, PCT Publication No. WO 2021/113702 all of which are incorporated by reference herein in their entirety. In aspects, the ILT7 to which the ILT7 binding protein binds is located on pDCs. In aspects, clone ILT70137 in PCT Publication No. WO 2017/156298 is provided and further described below in Table 1. In aspects, the anti-ILT7 binding protein comprises a sequence that comprises at least 85%, 90%, 95%, 96%, 97%, 98% or 99% identity to any one of the sequences of Table 1. In aspects, the anti-ILT7 binding protein comprises any one of the sequences of Table 1. daxdilimab is an antibody comprising a heavy chain variable region (VH) of SEQ ID NO: 1 and a light chain variable region (VL) of SEQ ID NO:2. VIB7734 and HZN7734 refer to Daxdilimab.

TABLE 1
Exemplary Anti-ILT7 mAb Sequence

| | SEQ | | |
|---|---|---|---|
| Clone | ID NO. | Description | Sequence |
| ILT70137 | 1 | Heavy chain | QVQLQQSGAEVKKPGASVKVSCKASGYTFTSYGI |
| | | variable | SWVRQAPGQGLEWMGWISAYNGNTNYAQKLQG |
| | | region (VH) | RVTMTTDTSTSTAYMELRSLRSDDTAVYYCARN |
| | | | GLWGWDSDAFDIWGRGTLVTVSS |
| | 2 | Light chain | QSALTQPASVSGSPGQSITISCTGTSSDVGGYNYV |
| | | variable | SWYQQHPGKAPKLMIYDVSNRPSGVSNRFSGSKS |
| | | region (VL) | GNTASLTISGLQAEDEADYYCSSYTSSSTVVFGGG |
| | | | TKVTVL |
| | 3 | HCDR1 | SYGIS |
| | 4 | HCDR2 | WISAYNGNTNYAQKLQG |
| | 5 | HCDR3 | NGLWGWDSDAFDI |
| | 6 | LCDR1 | TGTSSDVGGYNYVS |
| | 7 | LCDR2 | DVSNRPS |
| | 8 | LCDR3 | SSYTSSSTVV |
| | 9 | Light Chain | QSALTQPASVSGSPGQSITISCTGTSSDVGGYNYV |
| | | | SWYQQHPGKAPKLMIYDVSNRPSGVSNRFSGSKS |
| | | | GNTASLTISGLQAEDEADYYCSSYTSSSTVVFGGG |
| | | | TKVTVLGQPKAAPSVTLFPPSSEELQANKATLVCL |
| | | | ISDFYPGAVTVAWKADSSPVKAGVETTTPSKQSN |
| | | | NKYAASSYLSLTPEQWKSHRSYSCQVTHEGSTVE |
| | | | KTVAPTECS |
| | 10 | Heavy | QVQLQQSGAEVKKPGASVKVSCKASGYTFTSYGI |
| | | Chain | SWVRQAPGQGLEWMGWISAYNGNTNYAQKLQG |
| | | | RVTMTTDTSTSTAYMELRSLRSDDTAVYYCARN |
| | | | GLWGWDSDAFDIWGRGTLVTVSSASTKGPSVFPL |
| | | | APSSKSTSGGTAALGCLVKDYFPEPVTVSWNSGA |
| | | | LTSGVHTFPAVLQSSGLYSLSSVVTVPSSSLGTQT |
| | | | YICNVNHKPSNTKVDKRVEPKSCDKTHTCPPCPA |
| | | | PELLGGPSVFLFPPKPKDTLMISRTPEVTCVVVDV |
| | | | SHEDPEVKFNWYVDGVEVHNAKTKPREEQYNST |
| | | | YRVVSVLTVLHQDWLNGKEYKCKVSNKALPAPI |
| | | | EKTISKAKGQPREPQVYTLPPSREEMTKNQVSLTC |
| | | | LVKGFYPSDIAVEWESNGQPENNYKTTPPVLDSD |
| | | | GSFFLYSKLTVDKSRWQQGNVFSCSVMHEALHN |
| | | | HYTQKSLSLSPGK |

In aspects, the ILT7 binding protein comprises a VH and a VL at least 85%, 90%, 95%, 96%, 97%, 98% or 99% identical to: SEQ ID NO: 1 and SEQ ID NO: 2, respectively. In aspects, the VH and VL of an ILT7 binding protein comprises SEQ ID NO: 1 and SEQ ID NO: 2, respectively. In aspects, the VH and VL of an ILT7 binding protein comprises a sequence with at least about 85%, 90%, 95%, 96%, 97%, 98% or 99% identity to SEQ ID NO: 1 and/or SEQ ID NO: 2, respectively. In aspects, an isolated ILT7 binding protein is an ILT7 binding protein that can bind to the same ILT7 epitope as an antibody comprising a VH and a VL selected from the group consisting of SEQ ID NO: 1 and SEQ ID NO: 2, respectively. In aspects, an isolated ILT7 binding protein is an ILT7 binding protein that competitively inhibits the binding to ILT7 of an antibody comprising a VH and VL selected from the group consisting of SEQ ID NO: 1 and SEQ ID NO: 2, respectively. In aspects, an isolated ILT7 binding protein comprises a VH comprising SEQ ID NO: 1. In aspects, an ILT7 binding protein comprises a VH comprising a sequence having at least about 85%, 90%, 95%, 96%, 97%, 98% or 99% identity to SEQ ID NO: 1.

In aspects, an isolated ILT7 binding protein comprises a VL comprising SEQ ID NO: 2. In aspects, an ILT7 binding protein comprises a VL comprising a sequence having at least about 85%, 90%, 95%, 96%, 97%, 98% or 99% identity to SEQ ID NO: 2.

In aspects, an isolated ILT7 binding protein is an ILT7 binding protein comprising CDRs: HCDR1, HDR2, HCDR3, LCDR1, LCDR2, and LCDR3 selected from the group consisting of SEQ ID NOs: 3-8, respectively. In aspects, an isolated ILT7 binding protein is an ILT7 binding protein comprising CDRs: HCDR1, HDR2, HCDR3, LCDR1, LCDR2, and LCDR3 selected from the group consisting of SEQ ID NOs: 3-8, respectively, with at least 1-2, 2-3, or 2-4 residue modifications.

In aspects, the ILT7 binding protein comprises or consists of a VH at least 80%, 85%, 90%, 95%, 96%, 97%, 98% or 99% identical to SEQ ID NO: 1 and/or a VL at least 80%, 85%, 90%, 95%, 96%, 97%, 98% or 99% identical to SEQ ID NO:2. In aspects, an isolated ILT7 binding protein is an ILT7 binding protein comprising Complementarity-Determining Regions (CDRs) HCDR1, HDR2, HCDR3, LCDR1, LCDR2, and LCDR3 comprising the sequences of SEQ ID NOs: 3-8, respectively.

In aspects, the ILT7-binding proteins used in the methods described herein comprise heavy chain Complementarity-Determining Regions (HCDRs), HCDR1, HDR2, HCDR3, and light chain Complementarity Determining Regions (LCDRs), LCDR1, LCDR2, and LCDR3 having the amino acid sequences of SEQ ID NOs: 3, 4, 5, 6, 7, and 8, respectively. In other aspects, the ILT7-binding proteins used in the methods described herein comprise heavy chain Complementarity-Determining Regions (HCDRs), HCDR1, HDR2, HCDR3, and light chain Complementarity Determining Regions (LCDRs), LCDR1, LCDR2, and LCDR3, that are at least 85%, 90%, 95%, 96%, 97%, 98% or 99% identical to SEQ ID NOs: 3, 4, 5, 6, 7, and 8, respectively.

In aspects, the ILT7-binding proteins used in the methods described herein comprise a heavy chain of SEQ ID NO:9. In other aspects, the ILT7-binding proteins used in the methods described herein comprise a light chain of SEQ ID NO:10. In aspects, the ILT7-binding proteins used in the methods described herein comprise a heavy chain of SEQ ID NO:9 and a light chain of SEQ ID NO:10. In other aspects, the ILT7-binding proteins used in the methods described herein comprise a heavy chain that is at least 85%, 90%, 95%, 96%, 97%, 98% or 99% identical to SEQ ID NO:9 and/or a light chain that is at least 85%, 90%, 95%, 96%, 97%, 98% or 99% identical to SEQ ID NO:10.

In aspects, the ILT7 binding protein comprises a VH comprising SEQ ID NO: 1 and a VL comprising SEQ ID NO:2. In aspects, an isolated ILT7 binding protein is an ILT7 binding protein comprising a VH comprising SEQ ID NO:1. In aspects, an isolated ILT7 binding protein is an ILT7 binding protein comprising a VL comprising SEQ ID NO:2. In aspects, the ILT7 binding protein comprises a sequence with at least about 80%, 85%, 90%, 95%, 96%, 97%, 98% or 99% identity to SEQ ID NO: 9 and/or SEQ ID NO: 10. In aspects, the ILT7 binding protein comprises or consists of SEQ ID NO: 9 and/or SEQ ID NO: 10. In aspects, the ILT7 binding protein is Daxdilimab, a human immunoglobulin (Ig) G1 lambda (IgG12) afucosylated monoclonal antibody (mAb). Daxdilimab binds to ILT7 on the surface of pDCs, which leads to recruitment of macrophages and natural killer (NK) cells, thus inducing apoptosis and depletion of pDCs in vivo. The afucosylation of Daxdilimab is designed to enhance the potency of Daxdilimab for antibody-dependent cellular cytotoxicity (ADCC) against pDCs. Since pDCs are the major cell type that secretes type I interferons (IFNs) in response to nucleic acid-containing immune complexes, it is hypothesized that depletion of pDCs will reduce disease activity for subjects with autoimmune diseases (e.g., alopecia acreata) that are partially driven by abnormally high levels of type I IFNs.

In aspects, the ILT7 binding protein has ADCC activity against plasmacytoid dendritic cells (pDCs) in PMBCs.

In aspects, the ILT7 binding protein comprises a murine, human, chimeric, humanized, or resurfaced antibody or antigen-binding fragment thereof. In aspects, the ILT7 binding protein comprises an antibody, Fab, Fab′, F(ab′)2, Fd, single chain Fv or scFv, disulfide linked Fv, V-NAR domain, IgNar, intrabody, IgGACH2, minibody, F(ab′)3, tetrabody, triabody, diabody, single-domain antibody, DVD-Ig, Fcab, mAb2, (scFv)2, or scFv-Fc. In aspects, the ILT7 binding protein comprises a monoclonal antibody or an antigen binding fragment thereof. In aspects, the ILT7 binding protein comprises Daxdilimab. In aspects, the ILT7 binding protein is Daxdilimab.

In aspects, the ILT7 binding protein comprises a heavy chain immunoglobulin constant domain selected from the group consisting of: (a) an IgA constant domain; (b) an IgD constant domain; (c) an IgE constant domain; (d) an IgG1 constant domain; (e) an IgG2 constant domain; (f) an IgG3 constant domain; (g) an IgG4 constant domain; and (h) an IgM constant domain. In aspects, the ILT7 binding protein comprises a light chain immunoglobulin constant domain selected from the group consisting of: (a) an Ig kappa constant domain; and (b) an Ig lambda constant domain. In aspects, the ILT7 binding protein comprises a human IgG1 constant domain and a human lambda constant domain.

In aspects, provided herein is a host cell producing the ILT7 binding molecule.

In aspects, provided herein is an isolated polynucleotide comprising a nucleic acid sequence encoding a VH, wherein the VH comprises an amino acid sequence at least 85%, 90%, 95% identical, or identical to the VH of SEQ ID NO: 1.

In aspects, provided herein is an isolated polynucleotide comprising a nucleic acid sequence encoding a VL, wherein the VL comprises an amino acid sequence at least 85%, 90%, 95% identical, or identical to the VL of SEQ ID NO: 2.

In aspects, the nucleic acid is operably linked to a control sequence. In aspects, an antibody or antigen-binding fragment thereof comprising the VH or the VL encoded by the nucleic acid can specifically bind to ILT7.

In aspects, a polynucleotide encodes an ILT7 binding molecule provided herein.

In aspects, provided herein is a vector comprising the polynucleotide.

In aspects, provided herein is a polypeptide encoded by the polynucleotide.

In aspects, provided herein is a host cell transformed with a polynucleotide provided herein (e.g., a polynucleotide comprising a nucleic acid encoding a VH and a polynucleotide comprising a nucleic acid encoding a VL).

In aspects, provided herein is a host cell comprising a polynucleotide provided herein (e.g., a polynucleotide comprising a nucleic acid encoding a VH and a polynucleotide comprising a nucleic acid encoding a VL), a vector provided herein, or a polypeptide provided herein. In aspects, the host cell is a mammalian host cell. In aspects, the host cell is a NS0 murine myeloma cell, a PER.C6® human cell, or a Chinese hamster ovary (CHO) cells. In aspects, the host cell lacks the enzyme α-1,6-fucosyltransferase.

In aspects, the ILT7-binding proteins used in the methods described herein may contain fucose moieties or they may be afucosylated.

### Autoimmune Disease

In aspects, an autoimmune disease can be prevented or treated with any of the ILT7-binding proteins provided herein.

Examples of autoimmune disorders include but are not limited to systemic lupus erythematosus (SLE), discoid lupus erythematosus (DLE), lupus nephritis, cutaneous lupus erythematosus (CLE), Sjögren's syndrome, inflammatory myositis, Dermatomyositis, Anti-Synthetase Inflammatory Myositis (ASIM), inclusion body myositis, juvenile myositis and polymyositis, systemic sclerosis, diabetes, Hashimoto's disease, autoimmune adrenal insufficiency, pure red cell anemia, multiple sclerosis, rheumatic carditis, psoriasis, psoriatic arthritis, rheumatoid arthritis, chronic inflammation, chronic rheumatism, vitiligo, alopecia areata, hidradenitis suppurativa, celiac disease, acute and chronic graft versus host disease (GVHD), vascular inflammation, myocardial infarction, and type-1 interferonopathies. In aspects, the subject exhibits elevated type 1 IFNGS.

In aspects, the autoimmune disease is SLE. In further aspects, the autoimmune disease is CLE. In aspects, the autoimmune disease is lupus. In aspects, the lupus is not discoid lupus erythematosus (DLE). In aspects, the lupus is DLE. In aspects, the autoimmune disorder is primary DLE. In aspects, the subject with DLE does not have systemic lupus. In aspects, the DLE is refractory to other treatments. In aspects, the subject with DLE has previously been treated with one or more therapies selected from the group consisting of: a topical steroid, an intralesional steroids, an antimalarial, tacrolimus, thalidomide, optical tacrolimus, azathioprine, cyclosporin, mycophenolate mofetil, methotrexate, and acitretin.

In aspects, the autoimmune disease is discoid lupus erythematosus (DLE). Discoid lupus erythematosus generally manifests as disk-shaped, round lesions mostly on the scalp, face, and ears, and are often red, scaly, and thick. Over time, these can produce scarring, irreversible hair loss, and skin discoloration. Aside from hydroxychloroquine, there are no approved therapies for the treatment of DLE and current management consists of unapproved systemic therapy, like thalidomide, and local therapy with topical steroids or calcineurin inhibitors. Moreover, DLE is unresponsive to current standard of care is estimated to occur in approximately 30% of subjects and remains the most challenging scarifying skin manifestation to treat. Primary DLE lesions are known to contain increased numbers of pDCs, which have shown an elevated type I IFNs production. Interferon gene expression scores in the blood of DLE subjects are also significantly elevated compared to healthy controls. Moreover, PDCs express immunoglobulin-like transcript 7 (ILT7), a cell-surface protein unique to pDCs in human and the nonhuman primate.

In aspects, the autoimmune disease is Sjögren's syndrome. In aspects, the autoimmune disease is dermatomyositis. In aspects, the autoimmune disease is Dermatomyositis, In aspects, the autoimmune disease is Anti-Synthetase Inflammatory Myositis. In aspects, the autoimmune disease is polymyositis. In aspects, the autoimmune disease is systemic sclerosis. In aspects, the autoimmune disease is hidradenitis suppurativa. In aspects, the autoimmune disease is vitiligo.

In aspects, the autoimmune disease is alopecia areata. Alopecia areata is an acute onset autoimmune disorder characterized by transient, non-scarring hair loss in well-defined areas. It is the second most common form of Alopecia after the androgenetic type. The extent of hair loss defines the three main AA forms, namely Patchy AA, Alopecia Totalis (loss of all scalp hair) and Alopecia Universalis (loss of all body hair). Patchy AA is the most common form. Clinically, the patch of AA is usually completely bald, smooth, and round or oval in shape. In the periphery of the lesions, short, less than 4 mm exclamation mark hairs and black dots are commonly observed, especially in acute stages of AA. The scalp, the beard area and the eyebrow are most commonly involved, however any hair-bearing area can be affected. In aspects, alopecia does not destroy hair follicles.

The exclamation hair and the bald areas of alopecia areata can be caused by the inhibition of the hair follicle cycling, which represents one of the end results of the alopecia pathophysiologic process that starts with the loss of immune privilege of the hair follicle and a subsequent autoimmune attack of the bulbar region of anagen phase hair follicles by CD8+ T cells and Th1 cytokines. Genome-wide association studies have identified human leukocyte antigen (HLA) region genes and other immune function genes to be associated with alopecia areata. In aspects, CD8+NKG2D+T cells are effectors of AA pathogenesis. Effector CD8 T cells can get activated by expression of MHC I and NKG2DL on epithelial cells and can exert their cytotoxic effects through IFNg. Additionally, CD4 T cells and NK cells can also present around epithelial cells of afflicted subjects and secrete numerous pro-inflammatory cytokines, including IFNg. In aspects, the recruitment and activation of the peri- and intra-lesional NK and T cells can be caused by pDCs and/or Type I IFNs.

In aspects, the autoimmune disease is Dermatomyositis. In aspects, a subject with dermatomyositis comprises a skin rash, muscle weakness, dysphagia, tiredness, scaly skin, calcium deposits, inflammation, and combinations thereof.

In aspects, the autoimmune disease is Anti-Synthetase Inflammatory Myositis. In aspects, symptoms include inflammation of the muscles (myositis), inflammation of several joints (polyarthritis), interstitial lung disease, thickening and cracking of the skin of the hands, and a condition called Raynaud phenomenon, in which the fingers or toes are numb or have a prickly sensation in response to cold. Affected subjects also have nonspecific symptoms like fatigue, unexplained fevers, and unintended weight loss.

In aspects, the autoimmune disease is lupus nephritis (LN). LN is the most common major organ manifestation of SLE. LN affects approximately 31-60% of lupus patients and is more prevalent in certain ethnic groups, such as African Americans, Asians, and Hispanics. LN occurs when lupus autoantibodies affect structures in the kidneys that filter out waste. This causes kidney inflammation and may lead to blood in the urine, protein in the urine, high blood pressure, impaired kidney function, or even kidney failure.

In aspects, a subject with an autoimmune disease described herein comprises increased levels of interferon as compared to an otherwise comparable subject without the autoimmune disease or the same subject prior to diagnosis with the autoimmune disease. For example, in moderate to severe SLE about 80% of subjects are IFN^{hi}, even a higher percentage of moderate to severe dermatomyositis positive subjects, anti-synthetase myositis positive subjects, and lupus nephritis positive subjects are IFN^{hi}. Perhaps only about half of Sjogren's syndrome patients are IFN^{hi} however, and for RA it may only be about 15%. So there are subgroups of patients in a variety of these diseases that are IFN^{hi}. In aspects, a majority of subjects with an autoimmune disease comprise IFN^{hi}

### Methods

Provided are methods that comprise an ILT7 binding protein. In aspects, methods comprise using an ILT7 binding protein in a method of treatment. In aspects, a method of treatment comprises administering an ILT7 binding protein to a subject with an autoimmune disease. In aspects, a method comprises treating a subject with an autoimmune disease that is IFN^{hi} as determined by an in vitro assay, wherein the treating comprises administration of an ILT7 binding protein of the disclosure.

Without having to be bound by theory, the ILT7-binding proteins used in the methods described herein induce antibody-dependent cell-mediated cytotoxicity (ADCC) activity against plasmacytoid dendritic cells (pDCs), thereby depleting pDCs. In aspects, ILT7-binding protein-mediated ADCC causes a reduction in circulating pDCs. In aspects, ILT7-binding protein-mediated ADCC causes a reduction in local or tissue pDCs. In aspects, the tissue in which the pDCS are reduced includes, but is not limited to, skin cells, skin biopsy samples, kidney cells, lung cells, liver cells, heart cells, brain cells, nervous tissue, thyroid cells, eye cells, skeletal muscle cells, cartilage, bone tissue, and cells from airway passages. In aspects, the tissue is a skin biopsy sample. In more specific aspects, administering the ILT7-binding proteins will cause a reduction in skin pDCs. In aspects, the tissue in which the pDCS are reduced includes, but is not limited to scalp, eyebrow, nail, and eyelash. In aspects, the tissue is a scalp biopsy sample. In aspects, administering the ILT7-binding proteins will cause a reduction in pDCs in a tissue comprising a hair follicle.

Normally, pDCs are not present in skin tissue, and immature pDCs are typically only found in blood, thymus lymphoid tissue, tonsils and lung tissue. Thus the presence of pDCs in skin biopsy samples is indicative of an abnormal condition in which pDCs are recruited to the skin. Accordingly, the methods of the present disclosure include administering an ILT7-binding protein to a subject in need of treatment of a condition marked by the presence of pDCs in the subject's skin. The methods of the present disclosure include reducing the levels of pDCs in a subject's skin by administering an ILT7-binding protein to the subject in need of treatment thereof.

In aspects, the presence of pDCs in skin (e.g., scalp) biopsy samples is indicative of an abnormal condition in which pDCs are recruited to the skin. Accordingly, the methods of the present disclosure include administering an ILT7-binding protein to a subject in need of treatment of a condition marked by the presence of pDCs in a subject's skin. In certain aspects, the pDCs are located in a subject's hair follicle. In aspects, the pDCs are located at the bottom of the hair follicle. The methods of the present disclosure include reducing the levels of pDCs in a subject's skin by administering an ILT7-binding protein to a subject in need of treatment thereof.

In aspects, subjects have an elevated or high level of pDCs in skin tissue prior to treatment. In aspects, subjects with a high pDC level in skin tissue prior to treatment are more responsive to the treatment. In aspects, the subjects with a high pDC level in skin tissue have a pDC level of at least about 50 pDC/mm² of skin tissue, at least about 60 pDC/mm² of skin tissue, at least about 70 pDC/mm² of skin tissue, at least about 80 pDC/mm² of skin tissue, at least about 90 pDC/mm² of skin tissue, at least about 100 pDC/mm² of skin tissue, at least about 110 pDC/mm² of skin tissue, at least about 120 pDC/mm² of skin tissue, at least about 125 pDC/mm² of skin tissue, at least about 150 pDC/mm² of skin tissue, at least about 175 pDC/mm² of skin tissue, at least about 200 pDC/mm² of skin tissue, or higher. In aspects, a low pDC level in skin tissue is considered ≤10 pDC/mm² of skin tissue. In aspects a high pDC level in skin tissue is considered at least about 100 pDC/mm² of skin tissue.

In aspects, the methods of the present disclosure comprise administering an ILT7-binding proteins used in the methods described herein to suppress release of type I IFN from pDCs, regardless of the location of the pDCs. In other aspects, the methods of the present disclosure comprise administering an ILT7-binding protein to suppress release of type I IFN from pDCs in the blood or circulation. In other aspects, the methods of the present disclosure comprise administering an ILT7-binding protein to suppress release of type I IFN from local pDCs. In other aspects, the methods of the present disclosure comprise administering an ILT7-binding protein to suppress release of type I IFN from pDCs in the skin of the subject. In aspects, the type I IFN that suppressed in its release is IFNα. In aspects, ILT7-binding protein-mediated suppression of release of type I IFN from pDCs causes a reduction in type I IFNGS.

In aspects, when an autoimmune disease is alopecia treatment includes the application or administration of the ILT7-binding protein used in the methods described herein to a subject in need thereof or to a subject that is suspected of needing treatment thereof, or application or administration of the ILT7-binding protein used in the methods described herein to an isolated tissue (e.g., scalp, eyebrow, nail, and/or eyelash), where the subject has a disease, a symptom of a disease, or a predisposition toward a disease (e.g., alopecia areata). A subject may be suspected of needing the treatments described herein when the subject is exhibiting symptoms of a condition or disease by excess pDC numbers or activity, even though a formal diagnosis has not been ascertained. In aspects, a subject in need of treatment has a high baseline blood type I IFNG level as compared to an otherwise comparable subject that is not in need of treatment or otherwise healthy. In aspects, treatment is also intended to include the application or administration of a pharmaceutical composition comprising a ILT7-binding protein used in the methods described herein to a subject in need thereof or to a subject that is suspected of needing treatment thereof, or application, or administration of a pharmaceutical composition comprising a ILT7-binding protein used in the methods described herein to an isolated tissue or cell line from a subject who has a disease, a symptom of a disease, or a predisposition toward a disease (e.g., alopecia areata). In aspects, treatment includes the application or administration of the ILT7-binding protein used in the methods described herein to a subject in need thereof or to a subject that is suspected of needing treatment thereof, or application or administration of the ILT7-binding protein used in the methods described herein to an isolated tissue (e.g., scalp, eyebrow, nail, and/or eyelash), where the subject has a disease, a symptom of a disease, or a predisposition toward a disease (e.g., alopecia areata). A subject may be suspected of needing the treatments described herein when the subject is exhibiting symptoms of a condition or disease by excess pDC numbers or activity, even though a formal diagnosis has not been ascertained. In aspects, a subject in need thereof has a high baseline blood type I interferon level (e.g., IFNα) as compared to an otherwise comparable healthy subject. In aspects, treatment is also intended to include the application or administration of a pharmaceutical composition comprising a ILT7-binding protein used in the methods described herein to a subject in need thereof or to a subject that is suspected of needing treatment thereof, or application, or administration of a pharmaceutical composition comprising a ILT7-binding protein used in the methods described herein to an isolated tissue or cell line from a subject who has a disease, a symptom of a disease, or a predisposition toward a disease (e.g., alopecia areata). In aspects, the methods provided herein comprise evaluating the effect of Daxdilimab as compared with placebo to reduce hair loss in a subject with alopecia areata. The evaluating can occur at any time. In aspects, the evaluating occurs before administration, concurrent with administration, or after administration. In aspects, the evaluating occurs at week 1, 2, 3, 4, 5, 6, 7, 8, 9, 10, 20, 30, 40, 50, or 60 after administration of the ILT7 binding protein. In aspects, the evaluating occurs at weeks 12-36 and/or 40-48. In aspects, administration of an ILT7 binding protein is effective in reducing hair loss in a treated subject as compared to an otherwise comparable subject lacking the administration. Administration of an ILT7 binding protein is effective in reducing hair loss in a treated subject through about 1, 2, 3, 4, 5, 6, 7, 8, 9, 10, 11, 1 year, 1.5 years, 2 years, 3 years, 4 years, or up to about 5 years. In aspects, a treated subject experiences reduced hair loss through about 9 months of treatment with an ILT7 binding protein of the disclosure.

The disclosure provides methods of treating a subject with elevated type I IFNGS comprising administering the ILT7 binding proteins described herein. Subjects may exhibit an elevated type I IFNGS when suffering from an autoimmune disorder. Accordingly, the present disclosure provides methods of treating an autoimmune disorder when the subject is exhibiting an elevated type I IFNGS. In aspects, the autoimmune disorder is otherwise asymptomatic. In aspects, the methods provide selecting a subject for treatment with an ILT7-binding protein, the method comprising: (i) determining the baseline blood type I IFNGS level of the subject, and (ii) selecting those subjects with high baseline blood type I IFNGS levels for treatment with the ILT7-binding protein.

In aspects, the type I IFNGS is a 21-gene signature. In aspects, the type I IFNGS in the subject is at elevated by at least 1.5-fold relative to a normal score prior to treatment. In aspects, the type I IFNGS in the subject is at elevated by at least 2-fold relative to a normal score prior to treatment. In aspects, subjects with elevated type I IFNGS prior to treatment are more responsive to the treatment. In aspects, type I IFNGS is at least about 4-fold, at least about 5-fold, at least about 6-fold, at least about 7-fold, at least about 8-fold, at least about 9-fold, at least about 10-fold, at least about 11-fold, at least about 12-fold or higher relative to a normal score prior to treatment with an ILT7-binding protein used in the methods described herein. In particular aspects, the tissue type I IFNGS is determined from a skin biopsy. In other aspects, the tissue type I IFNGS is determined using the IFN-inducible Myxovirus protein A (MxA) immunohistochemistry (IHC) test. In further aspects, the IFN-inducible gene expression in the epidermis is determined using skin tape stripping, RNA isolation and gene expression profiling (https://dermtech.com/wp-content/uploads/Lupus-Reference.pdf).

In aspects, the disclosure is directed to a method for reducing a type I interferon gene signature (IFNGS) in a subject in need thereof, the method comprising administering to the subject a pharmaceutically effective amount of an immunoglobulin-like transcript 7 (ILT7)-binding protein, wherein the ILT7 binding protein is administered to the subject when the type I IFNGS is elevated in the subject relative to the type I IFNGS in a normal subject. In aspects, the pharmaceutically effective amount of the ILT7-binding protein is about 200 mg.

In aspects, the type I IFNGS comprises expression levels of all type I IFN-inducible genes in a biological sample. In other aspects, the type I IFNGS comprises expression levels of a subset of type I IFN-inducible genes in a biological sample.

In aspects, the type I IFNGS is determined by assaying the expression levels of at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 200, at least 300, at least 400, or at least 500 type I IFN-inducible genes in a biological sample. In aspects, the type I IFNGS comprises the collective expression levels of two or more type I IFN-inducible genes. In aspects, the two or more type I interferon (IFN)-inducible genes include, but are not limited to, two or more genes chosen from SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, or USP18. In aspects, the type I IFNGS is determined by assaying the collective expression levels of SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18. These gene symbols are well-known in the art and refer to human and non-human orthologs of the listed genes.

TABLE 2
Gene Symbols and Their Correlating Names.

| Gene Symbol | Correlating Name |
|---|---|
| SPATS2L | Spermatogenesis Associated Serine Rich 2 Like |
| EPSTI | Epithelial Stromal Interaction 1 |
| HERC5 | HECT and RLD Domain Containing E3 |
| | Ubiquitin Protein Ligase 5 |
| IFI27 | Interferon Alpha Inducible Protein 27 |
| IFI44 | Interferon Induced Protein 44 |
| IFI44L | Interferon Induced Protein 44-like |
| IFI6 | Interferon Alpha Inducible Protein 6 |
| IFIT1 | Interferon Induced Protein With |
| | Tetratricopeptide Repeats 1 |
| IFIT3 | Interferon Induced Protein With |
| | Tetratricopeptide Repeats 3 |
| ISG15 | Interferon Stimulated gene 15 |
| LAMP3 | Lysosomal Associated Membrane Protein 3 |
| LY6E | Lymphocyte Antigen 6 Family Member E |
| MX1 | MX Dynamin Like GTPase 1 |
| OAS1 | 2′-5′-Oligoadenylate Synthetase 1 |
| OAS2 | 2′-5′-Oligoadenylate Synthetase 2 |
| OAS3 | 2′-5′-Oligoadenylate Synthetase 3 |
| PLSCR1 | Phospholipid Scramblase 1 |
| RSAD2 | Radical S-Adenosyl Methionine Domain Containing 2 |
| RTP4 | Receptor Transporter Protein 4 |
| SIGLEC1 | Sialic Acid Binding Ig Like Lectin 1 |
| USP18 | Ubiquitin Specific Peptidase 18 |

In aspects, the expression levels of the type I interferon (IFN)-inducible genes are determined by measuring the DNA levels (e.g., complementary DNA or cDNA levels) of the type I interferon (IFN)-inducible genes in a biological sample. In aspects, the expression levels of the type I interferon (IFN)-inducible genes are determined by measuring the messenger RNA (mRNA) levels of the type I interferon (IFN)-inducible genes in a biological sample. In aspects, the type I IFNGS comprises mRNA levels of all type I IFN-inducible genes in the biological sample. In other aspects, the type I IFNGS comprises mRNA levels of a subset of type I IFN-inducible genes in the biological sample taken from a subject affected, likely to be affected, or suspected to be affected with a disease, e.g., an autoimmune disease. In aspects, the type I IFNGS is determined by assaying the mRNA levels of the two or more type I interferon (IFN)-inducible genes in a biological sample. In aspects, the type I IFNGS is determined by assaying the mRNA levels of the 21 type I interferon (IFN)-inducible genes in a biological sample (e.g. SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18). In aspects, the biological sample is a test biological sample. In other aspects, the biological sample is a normal biological sample.

In aspects, the type I IFNGS is measured in test biological samples taken from the subject. In other aspects, the pDCs are measured in test biological samples taken from the subject. The biological sample includes, but is not limited to, blood, sputum, saliva, skin cells, skin biopsy samples, kidney cells, lung cells, liver cells, heart cells, brain cells, nervous tissue, thyroid cells, eye cells, skeletal muscle cells, cartilage, bone tissue, cells from airway passages, and cultured cells. In aspects, the biological sample is blood. In other aspects, the biological sample is tissue. In more specific aspects, the sample is a tissue comprising skin cells. In other aspects, the sample is a skin biopsy sample.

In aspects, administration of a therapeutically effective amount of an ILT7-binding protein used in the methods described herein to a subject in need thereof leads to about 1% to about 100% reduction in type I IFNGS in the subject compared to the type I IFNGS prior to administration of the ILT7-binding protein used in the methods described herein. In aspects, administration of a therapeutically effective amount of an ILT7-binding protein used in the methods described herein to a subject in need thereof leads to at least about 1%, at least about 2%, at least about 5%, at least about 10%, at least about 20%, at least about 30%, at least about 30%, at least about 40%, at least about 50%, at least about 60%, at least about 70%, at least about 80%, at least about 90%, at least about 95%, at least about 96%, at least about 97%, at least about 98%, at least about 99%, or about 100% reduction in type I IFNGS in the subject, compared to the type I IFNGS prior to administration of the ILT7-binding protein used in the methods described herein. In aspects, administration of a therapeutically effective amount of the ILT7-binding protein leads to at least about 50% reduction in the type I IFNGS in the subject.

In aspects, administration of the ILT7-binding protein used in the methods described herein to a subject in need thereof leads to at least about 50% reduction in the type I IFNGS in the subject, compared to the type I IFNGS prior to administration of the ILT7-binding protein used in the methods described herein. In aspects, administration of a therapeutically effective amount of an ILT7-binding protein used in the methods described herein to a subject in need thereof leads to the at least about 50% reduction in type I IFNGS in the subject at about 8 hours, about 12 hours, about 24 hours, or about 48 hours following administration of the ILT7-binding protein.

In aspects, a subject who has been administered a therapeutically effective amount of an ILT7-binding protein used in the methods described herein shows a reduction in type I IFNGS of at least about 50% at about 24 hours following administration of the ILT7-binding protein, compared to the type I IFNGS in the subject prior to administration of the ILT7-binding protein.

In aspects, the reduction in type I IFNGS persists for at least about 1 day, at least about 2 days, at least about 3 days, at least about 4 days, at least about 5 days, at least about 6 days, at least about 7 days, at least about 14 days, at least about 21 days, at least about 28 days, at least about 30 days, at least about 45 days, at least about 60 days, at least about 90 days, or at least about 180 days or longer following administration of a therapeutically effective amount of an ILT7-binding protein used in the methods described herein to a subject in need thereof. In aspects, the reduction in type I IFNGS persists for up to about 30 days following administration of a therapeutically effective amount of an ILT7-binding protein used in the methods described herein to a subject in need thereof. In additional aspects, the reduction in type I IFNGS persists for up to about 60 days following administration of a therapeutically effective amount of an ILT7-binding protein used in the methods described herein to a subject in need thereof.

In aspects, the methods of the present disclosure can be used to monitor the effectiveness of treatment of conditions or disorders by monitoring levels of type I IFNGS and/or activated pDCs. As noted above, autoimmune conditions are often marked by elevated type I IFNGS and/or elevated pDCs, thus monitoring the effectiveness of treatments can include monitoring type I IFNGS and/or pDC levels.

Thus, in aspects, the disclosure provides a method of monitoring effectiveness of treatment of an autoimmune disorder or condition, comprising the steps of: (a) measuring a type I interferon gene signature (IFNGS) in a biological sample taken from the subject to obtain a baseline value of the type I IFNGS; and (b) measuring the type I IFNGS in a biological sample taken from the subject after administering a treatment, wherein the treatment comprises administering an ILT7-binding protein, and wherein a decrease in the type I IFNGS in step (b) compared to the baseline value indicates that the treatment is effective in the subject.

In aspects, the treatment results in a decrease in the type I IFNGS compared to the baseline value. In aspects, the decrease in the type I IFNGS compared to the baseline value ranges from about 1% to about 99%. In aspects, the decrease in the type I IFNGS compared to the baseline value is at least about 5%, at least about 10%, at least about 20%, at least about 30%, at least about 40%, at least about 50%, at least about 60%, at least about 70%, at least about 80%, at least about 90%, at least about 95%, or at least about 99%. In aspects, the decrease in the type I IFNGS compared to the baseline value is at least about 30%. In aspects, the decrease in the type I IFNGS compared to the baseline value is at least about 50%.

In aspects, the elevation in type I IFNGS in a test biological sample relative to a normal biological sample, or in a subject in need of treatment with an ILT7 binding protein relative to a normal subject is at least a fold change of about 1.1 to about 1000. Thus, in aspects, the type I IFNGS is elevated by at least about 1.1, 1.2, 1.3, 1.4, 1.5, 2, 3, 4, 5, 6, 7, 8, 9, 10, 15, 20, 30, 40, 50, 60, 70, 80, 90, or 100-fold in a test biological sample relative to a normal biological sample, or in a subject in need of treatment with an ILT7 binding protein relative to a normal subject. In aspects, the type I IFNGS is elevated by at least about 4-fold in the test biological sample relative to the normal biological sample, or in a subject in need of treatment with an ILT7 binding protein relative to a normal subject.

In aspects, the disclosure is directed to a method of reducing plasmacytoid dendritic cells (pDCs) in a tissue of a subject in need thereof, the method comprising administering to the subject a pharmaceutically effective amount of an immunoglobulin-like transcript 7 (ILT7)-binding protein. In aspects, the pharmaceutically effective amount of the ILT-7 binding protein is 150 mg. In aspects, the pharmaceutically effective amount of the ILT-7 binding protein is 200 mg. In aspects, the pharmaceutically effective amount of the ILT-7 binding protein is 300 mg. In aspects, the ILT7-binding protein is administered once about every four weeks. In aspects, the ILT7-binding protein is administered once about every twelve weeks. In aspects, the ILT7-binding protein is administered once about every twelve weeks and the subject is administered an additional 200 mg dose of the ILT7-binding protein about four weeks after the initial dose. In aspects, the disclosure is directed to a method of reducing plasmacytoid dendritic cells (pDCs) in a tissue of a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, and wherein the subject is administered: a first dose, a second dose about four weeks after the first dose, a third dose about twelve weeks after the first dose, and subsequent doses about twelve weeks after the third dose.

In aspects, the disclosure is directed to a method of reducing plasmacytoid dendritic cells (pDCs) in a tissue of a subject in need thereof, the method comprising administering to the subject a pharmaceutically effective amount of an immunoglobulin-like transcript 7 (ILT7)-binding protein. In aspects, the pharmaceutically effective amount of the ILT7-binding protein is about 100 mg, 150 mg, 200 mg, 250 mg, or 300 mg. In aspects, the ILT7-binding protein is administered once about every four weeks, every twelve weeks, or combinations thereof. In aspects, the ILT7-binding protein is administered every week, every two weeks, every three weeks, every four weeks, every twelve weeks, or combinations thereof. In aspects, the ILT-7 binding protein is daxdilimab. In aspects, the autoimmune disease is lupus. In aspects, the autoimmune disease is SLE. In aspects, the autoimmune disease is DLE. In aspects, the autoimmune disease is alopecia areata. In aspects, the autoimmune disease is dermatomyositis.

In aspects, the reduction in pDCs in the subject is about 1% to about 100% compared to the pDCs in the subject prior to administration of an ILT7-binding protein used in the methods described herein. In aspects, the a reduction in pDCs in the subject is at least about 1%, at least about 2%, at least about 5%, at least about 10%, at least about 20%, at least about 30%, at least about 30%, at least about 40%, at least about 50%, at least about 60%, at least about 70%, at least about 80%, at least about 90%, at least about 95%, at least about 96%, at least about 97%, at least about 98%, at least about 99%, or about 100% compared to pDCs in the subject prior to administration of an ILT7-binding protein used in the methods described herein. In aspects, the reduction in pDCs in the subject is at least about 50% compared to pDCs in the subject prior to administration of an ILT7-binding protein used in the methods described herein. Thus, in aspects, administration of a therapeutically effective amount of the ILT7-binding protein leads to at least about 10% reduction in total number of pDCs in the subject. In additional aspects, administration of a therapeutically effective amount of the ILT7-binding protein leads to at least about 10% reduction in activated pDCs in the subject. In aspects, the pDCs are measured in a test biological sample taken from the subject. Therefore, in aspects, administering a therapeutically effective amount of an ILT7-binding protein used in the methods described herein to a subject in need thereof leads to a reduction in pDCs in a test biological sample taken from the subject. In aspects, the reduction in pDCs in a test biological sample taken from the subject is at least about 10% compared to pDCs in the test biological sample prior to administration of an ILT7-binding protein used in the methods described herein. In aspects, the test biological sample is blood. In aspects, the test biological sample is tissue, including, but not limited to, skin cells and skin biopsy specimens. In aspects, the pDCs are circulating pDCs. In other aspects, the pDCs are pDCs in the skin. In additional aspects, the reduction in pDCs is reversible.

In aspects, administration of a therapeutically effective amount of an ILT7-binding protein used in the methods described herein to a subject in need thereof causes at least about 10% reduction in pDCs in the subject at about 5 minutes, at about 10 minutes, about 15 minutes, about 30 minutes, about 1 hour, about 2 hours, about 3 hours, about 6 hours, about 12 hours, about 24 hours, or about 48 hours following administration of the ILT7-binding protein. In other aspects, administration of a therapeutically effective amount of an ILT7-binding protein used in the methods described herein to a subject in need thereof causes at least about 10% reduction in pDCs in a test biological sample taken from the subject at about 5 minutes, at about 10 minutes, about 15 minutes, about 30 minutes, about 1 hour, about 2 hours, about 3 hours, about 6 hours, about 12 hours, about 24 hours, or about 48 hours following administration of the ILT7-binding protein. In aspects, administration of a therapeutically effective amount of an ILT7-binding protein used in the methods described herein to a subject in need thereof causes at least about 10% reduction in pDCs in the subject at about 1 week, 2 weeks, 3 weeks, 1 month, 3 months, 5 months, 8 months, 12 months, or 1.5 years following administration of the ILT7-binding protein. In aspects, administration of a therapeutically effective amount of an ILT7-binding protein used in the methods described herein to a subject in need thereof causes at least about 10% reduction in pDCs in a test biological sample taken from the subject at about 12 hours, about 24 hours, or about 48 hours following administration of the ILT7-binding protein.

In aspects, the reduction in pDCs persists for at least about 1 day, at least about 2 days, at least about 3 days, at least about 4 days, at least about 5 days, at least about 6 days, at least about 7 days, at least about 14 days, at least about 21 days, at least about 28 days, at least about 30 days, at least about 45 days, at least about 60 days, at least about 90 days, or at least about 180 days or longer following administration of a therapeutically effective amount of an ILT7-binding protein used in the methods described herein to a subject in need thereof. In aspects, the reduction in pDCs persists for at least about 30 days following administration of a therapeutically effective amount of an ILT7-binding protein used in the methods described herein to a subject in need thereof. In additional aspects, the reduction in pDCs persists for at least about 60 days following administration of a therapeutically effective amount of an ILT7-binding protein used in the methods described herein to a subject in need thereof.

In aspects, provided is a method for preventing or treating dermatomyositis. In aspects, a method comprises administering an effective amount of a composition comprising daxdilimab to treat dermatomyositis. In aspects, administration is effective in reducing or eliminating a symptom of dermatomyositis selected from the group consisting of: skin rash, muscle weakness, dysphagia, tiredness, scaly skin, calcium deposits, and inflammation. In aspects, the symptom is reduced by at least about 5%, 10%, 20%, 30%, 40%, 50%, 60%, 70%, 90%, 120%, 140%, 160%, 180%, or 200% as compared to an otherwise comparable subject lacking the administering.

### Assessments

In aspects, the methods provided herein comprise evaluating the effect of daxdilimab as compared with placebo to reduce symptoms of or eliminate an autoimmune disease. Evaluation of an autoimmune disease can occur at any time. In aspects, the evaluating occurs before administration, concurrent with administration, or after administration of any composition provided herein. In aspects, the evaluating occurs at week 1, 2, 3, 4, 5, 6, 7, 8, 9, 10, 20, 24, 30, 40, 45, 48, 50, 52, 60, 64, 70, 80, 90, 100, 104, or 110 after administration of the daxdilimab. In aspects, the evaluating occurs at week 48 post administration.

In aspects, the methods provided herein comprise evaluating sustained oral glucocorticoid reduction after administration of daxdilimab. In aspects, evaluation comprises measuring the proportion of subjects at oral glucocorticoid ≥10 mg prednisone or equivalent at baseline (Day 1) who achieve an OCG of ≤7.5 mg/day prednisone or equivalent. The evaluating can occur at any time. In aspects, the evaluating occurs before administration, concurrent with administration, or after administration. In aspects, the evaluating occurs from week 1 to week 10, week 20-week 40, week 30-week 50, or from week 1-week 50. In aspects, the evaluating occurs from week 36 to week 48.

In other aspects, the methods of the present disclosure can be used for reducing Cutaneous Lupus Erythematosus Disease Activity and Severity Index (CLASI) in a tissue of a subject in need thereof. The methods comprise administering to the subject a pharmaceutically effective amount of an ILT7-binding protein. In aspects, the ILT7-binding protein is administered at a dose of 200 mg. In particular aspects, the ILT7-binding protein is administered once about every four weeks. In aspects, the ILT7-binding protein is administered once about every twelve weeks. In one aspect, the ILT7-binding protein is administered once about every twelve weeks and the subject is administered an additional 200 mg dose of the ILT7-binding protein about four weeks after the initial dose.

As used herein, the term “CLASI” refers to Cutaneous Lupus Erythematosus Disease Activity and Severity Index. The CLASI is a validated instrument for measuring skin manifestations of CLE. The CLASI consists of two scores: the first summarizes the inflammatory activity of the disease; the second is a measure of the damage done by the disease. The activity score includes erythema (0-3), scale/hypertrophy (0-2), mucous membrane lesions (0-1), recent hair loss (0-1) and non-scarring alopecia (0-3). The damage score represents dyspigmentation (0-1), scarring/atrophy/panniculitis (0-2), and scarring of the scalp (0-6). Subjects are asked if their dyspigmentation lasts 12 months or longer, in which case, the dyspigmentation score is doubled. Each of the above parameters is measured in 13 different anatomical locations, included specifically because they are most often involved in CLE. The most severe lesion in each area is measured.

As used herein, the term “reduction CLASI” refers diminished levels of CLASI-Activity (CLASI-A) score in a subject or in a biological sample (e.g., issues such as skin cells, skin biopsy samples, etc.) taken from the subject, or diminished levels of CLASI-Damage (CLASI-D) score in a subject or in a biological sample taken from the subject, or both.

In aspects, the methods of the disclosure comprise evaluating the effect of administration of daxdilimab as compared with placebo to reduce cutaneous disease activity. In aspects, evaluation comprises measuring a proportion of subjects with a CLASI-A score ≥10 at baseline (Day 1) who achieve ≥50% reduction from baseline (Day 1) in CLASI-A score. Scoring comprises evaluating at least one of: inflammatory activity of the disease and/or damage done by the disease. The evaluating can occur at any time. In aspects, the evaluating occurs before administration, concurrent with administration, or after administration. In aspects, the evaluating occurs at week 1, 2, 3, 4, 5, 6, 7, 8, 9, 10, 11, 12, 13, 14, or 15 after administration of the daxdilimab. In aspects, the evaluating occurs at week 12 post administration.

Thus, in aspects, the methods of the disclosure result in a reduced CLASI-A score in the subject. In aspects, a reduction in the CLASI-A score of a subject involves a reduction of the CLASI-A score by at least 1, 2, 3, 4, 5, 6, 7, 8, 9, or 10 points from a baseline value. In aspects, a reduction in the CLASI-A score of a subject involves a reduction of the CLASI-A score by at least 4 points from a baseline value. In aspects, a reduction in the CLASI-A score of a subject involves a reduction of the CLASI-A score by at least 7 points from a baseline value. In other aspects, a reduction in the CLASI-A score of a subject involves a reduction of the CLASI-A score by at least 5%, 10%, 15%, 20%, 25%, 30%, 35%, 40%, 45%, 50%, 55%, 60%, 65%, 70%, 75%, 80%, 85%, 90%, 95%, or 99% from a baseline value. In aspects, a reduction in the CLASI-A score of a subject involves a reduction of the CLASI-A score by at least 50% from a baseline value. In aspects, the baseline value is the value of the CLASI-A score in the subject prior to treatment with an ILT7-binding protein used in the methods described herein. In other aspects, the methods of the present disclosure result in a reduced CLASI-D score in the subject. In additional aspects, the methods of the present disclosure result in a reduced CLASI-A score and a reduced CLASI-D score in the subject.

In aspects, the methods comprise evaluating Lupus Low Disease Activity State (LLDAS) disease activity. In aspects, evaluation comprises measuring the proportion of subjects achieving LLDAS. LLDAS is a composite measure of SLE disease activity that measures 5 criteria: (1) SLEDAI-2K≤4, with no activity in major organ systems, (2) no new lupus disease activity, (3) PGA≤1 (scale 0 to 3), (4) current prednisone or equivalent dose≤7.5 mg daily, (5) tolerated maintenance doses of immunosuppressive drugs, and approved biological agents. The evaluating can occur at any time. In aspects, the evaluating occurs before administration, concurrent with administration, or after administration. In aspects, the evaluating occurs at week 1, 2, 3, 4, 5, 6, 7, 8, 9, 10, 20, 30, 40, 50, or 60 after administration of the daxdilimab. In aspects, the evaluating occurs at week 48 post administration.

In aspects, a subject in need thereof or a sample from a subject in need thereof is evaluated using an in vitro test. Suitable in vitro tests, include but are not limited to: SLE Disease Activity Index 2000 (SLEDAI-2K), British Isles Lupus Assessment Group (BILAG) 2004 Index, Physician Global Assessment (PGA), Classification Criteria for SLE, antinuclear antibody (ANA), Anti-dsDNA antibodies, Anti-Smith antibodies, and any combination thereof.

In aspects, a subject in need thereof comprises at least one or all of the following: Age≥18 years to ≤70 years at the time of signing the informed consent form (ICF); Willing and able to understand and provide written informed consent prior to any study-related procedures and to comply with all study requirements and complete study assessments; fulfill the 2019 European League Against Rheumatism/American College of Rheumatology Classification Criteria for SLE (Aringer et al, 2019); Disease duration of at least 6 months from the time of diagnosis at the time of signing the ICF; Active SLE as indicated by presence of all the following: SLEDAI-2K total score ≥6 at Screening, excluding fever, SLE headache, or organic brain syndrome, SLEDAI-2K total score ≥4, excluding points attributable to any urine or laboratory results, immunologic measures, fever, SLE headache, or organic brain syndrome at Screening and Baseline (Day 1), At least 1 of the following BILAG 2004 Index levels of disease at Screening: BILAG A disease in ≥1 organ system and/or BILAG B disease in ≥2 organ systems, PGA score ≥1 on a 0 to 3 visual analog scale (VAS) at Screening; have at least 1 of the following at Screening per central lab: antinuclear antibody (ANA)≥1:80, Anti-dsDNA antibodies elevated to above normal range as established by the central laboratory (i.e., positive results), Anti-Smith antibodies elevated to above normal (i.e., positive results), Ongoing treatment for SLE defined as (a) or (b): (a)Treatment with a disease modifying anti-rheumatic drug (DMARD) or immunosuppressive medication: Any of the following medications each administered at conventional anti-rheumatic doses for treatment of SLE for at least 12 weeks before screening (unless discontinued or dose adjusted for documented drug-related toxicity or size/weight), and at a stable dose (including route of administration) for a minimum of 8 weeks prior to Screening and maintained through Baseline (Day 1): (1) Antimalarial (Chloroquine, Hydroxychloroquine, Quinacrine), (2) Azathioprine (AZA) or 6-mercaptopurine (6-MP), (3) Leflunomide, (4) Mycophenolate mofetil (MMF) or mycophenolic acid (MPA), (5) Methotrexate (MTX) (subjects must be on concomitant folic or folinic acid supplementation if using MTX), (6) Voclosporin (if approved for treatment), and/or (7) GCs are permitted but not required if a subject is receiving at least 1 other medication listed above. If glucocorticoids are used in combination with allowed DMARDs or immunosuppressants, they may be at an average daily dose of PO prednisone ≤40 mg (or prednisone equivalent) for a minimum of 2 weeks prior to Screening and at a stable dose for a minimum of 2 weeks prior to screening. In addition, the dose of OGC must be kept stable for a minimum of 2 weeks prior to randomization. Daily dosing or alternate day dosing of PO prednisone (or prednisone equivalent) is allowed; and (b) Treatment with oral glucocorticoid monotherapy (without the concomitant use of DMARDs or immunosuppressants): Average daily dose of PO prednisone ≥10 mg but ≤40 mg (or prednisone equivalent) for a minimum of 4 weeks prior to screening and at a stable dose for a minimum of 2 weeks prior to Screening. In addition, the dose of OGC must be kept stable for a minimum of 2 weeks prior to Randomization. Daily dosing or alternate day dosing of PO prednisone (or prednisone equivalent) is allowed, women of childbearing potential must have a negative urine pregnancy test at Randomization. Women of childbearing potential are defined as those who are not surgically sterile (i.e., surgical sterilization includes bilateral tubal ligation, bilateral oophorectomy, or hysterectomy) or those who are not postmenopausal (defined as 12 months with no menses without an alternative medical cause and a follicle-stimulating hormone [FSH] within the postmenopausal range as established by the central laboratory, unless on postmenopausal hormone replacement therapy), women of childbearing potential who are sexually active with a non-sterilized male partner must agree to use a highly effective method of contraception from signing of the informed consent, and must agree to continue using such precautions through the end of the study follow-up or 3 months (approximately 5 half-lives) following the last dose of study drug in the case of early withdrawal from the study. A decision about contraception after this point should be made by the subject and her regular healthcare providers, sustained abstinence is an acceptable practice; however periodic abstinence, the rhythm method, and the withdrawal method are not acceptable methods of contraception. In aspects, because mycophenolate affects the metabolism of hormonal contraceptives and may reduce their effectiveness in women receiving MMF or MPA who are using hormonal contraceptives for birth control, the subject must employ an additional contraceptive method (e.g., barrier method), and/or non-sterilized male subjects who are sexually active with a woman partner of childbearing potential must agree to use a condom with spermicide from Randomization and until 3 months (approximately 5 half-lives) after receipt of the last dose. Because a male condom with spermicide is not a highly effective contraception method, it is strongly recommended that male subjects advise their women partners of childbearing potential to use a highly effective method of contraception throughout this period.

In aspects, a subject in need thereof may not have a history of allergy, hypersensitivity reaction, or anaphylaxis to any component of a provided composition, to a previous mAb, and/or to a human Ig therapy. In aspects, a subject is not a subject enrolled in another clinical study with an IP within 4 weeks prior to Day 1 or within 5 published half-lives, whichever is longer. In aspects, a subject is not lactating, pregnant, or intending to become pregnant anytime from signing the ICF through 6 months after receiving the last dose of IP. In aspects, a subject does not have a history of drug or alcohol abuse that, in the opinion of the Investigator, might affect subject safety or compliance with visits, or interfere with other study assessments. In aspects, a subject has not had a major surgery within 8 weeks prior to Screening or elective surgery planned from screening through day 393. In aspects, a subject has not had a spontaneous or induced abortion, still or live birth, or pregnancy ≤4 weeks prior to Screening. In aspects, a subject does not have a known history of a primary immunodeficiency or an underlying condition such as known human immunodeficiency virus (HIV) infection, a positive result for HIV infection per central laboratory, splenectomy, or any underlying condition that in the opinion of the Investigator significantly predisposes the subject to infection.

In aspects, a subject at screening, does not have any of the following per central laboratory (tests may be repeated once within the same Screening period to confirm results prior to Randomization): AST≥2.5×ULN; ALT≥2.5×ULN; Total bilirubin≥1.5×ULN (unless due to Gilbert's syndrome); Serum IgG≤600 mg/dL (or ≤6 g/L); Neutrophil count≤1000/μL (or ≤ 1.0×10⁹/L) or ≤500/μL (≤0.5×10⁹/L) if due to active SLE; Platelet count≤50,000/μL (or ≤ 50×10⁹/L) or ≤250,000/μL (≤25×10⁹/L) if due to active SLE; Hemoglobin≤8 g/dl (or ≤80 g/L) or ≤7 g/dL (≤70 g/L) if due to active SLE; Glycosylated hemoglobin≥8% (or ≥0.08); Total lymphocyte count≤200 cells/mm³; Glomerular filtration rate≤30 mL/min/1.73 m2; Spot UPCr≥3 mg/mg; Confirmed positive test for hepatitis B serology defined as: Hepatitis B surface antigen (HBsAg), or Hepatitis B core antibody (HBcAb) AND hepatitis B virus (HBV) DNA detected above the lower limit of quantitation (LLOQ) by reflex testing by the central laboratory at Screening. In aspects, a subject who is HBcAb positive at Screening are tested every 3 months for HBV DNA. In aspects, a study drug is discontinued if the subject's HBV DNA levels are confirmed to exceed the LLOQ as per the central laboratory. In aspects, a subject does not have a positive test for hepatitis C virus antibody. In aspects, a subject does not have an active TB, or a positive IFN-gamma release assay (IGRA) test at Screening, unless documented history of appropriate treatment for active or latent TB. In aspects, a subject with an indeterminate IGRA test result can repeat the test, but if the repeat test is also indeterminate, they are excluded from receiving a composition provided herein. In aspects, a subject does not have any severe herpes virus family infection (including Epstein-Barr virus, cytomegalovirus [CMV]) at any time prior to Randomization, including, but not limited to, disseminated herpes, herpes encephalitis, recent recurrent herpes zoster (defined as 2 episodes within the last 2 years), or ophthalmic herpes. In aspects, a subject does not have any herpes zoster, CMV, or Epstein-Barr virus infection that was not completely resolved 12 weeks prior to randomization.

In aspects, a subject does not have any of the following within 30 days prior to signing the ICF and though randomization: Clinically significant active infection in the opinion of the Investigator, including ongoing, and chronic infection requiring antibiotics or antiviral medication (chronic nail infections are allowed); any infection requiring hospitalization or treatment with IV anti-infectives; a documented positive SARS COV-2 test may be rescreened at least 2 weeks after a positive test if the subject is asymptomatic and at least 3 weeks after symptomatic COVID-19 illness; and/or an opportunistic infection requiring hospitalization or parenteral antimicrobial treatment within 2 years prior to Randomization.

In aspects, a subject does not have any acute illness or evidence of clinically significant active infection, such as fever≥38.0° C. (≥100.5° F.) on Day 1.

In aspects, a subject does not have a history of clinically significant cardiac disease including unstable angina; myocardial infarction within 6 months prior to Randomization; congestive heart failure; arrhythmia requiring active therapy, except for clinically insignificant extra systoles, or minor conduction abnormalities; or presence of clinically significant abnormality on ECG if, in the opinion of the Investigator, it would increase the risk of study participation. In aspects, a subject does not have a history of cancer within the past 5 years, except as follows: In situ carcinoma of the cervix treated with apparent success with curative therapy≥12 months prior to Screening, or Cutaneous basal cell or squamous cell carcinoma treated with apparent success with curative therapy.

In aspects, a subject has not received a live-attenuated vaccine within 4 weeks prior to Day 1. In aspects, a subject has not received an administration of inactivated (killed) vaccines. In aspects, a subject has received an administration of inactivated (killed) vaccines.

In aspects, a subject is assessed for epidemiologic risk of COVID-19 (i.e., recent exposure, high-risk housing) and for health-related risk of COVID-19 severity based on current understanding of risk factors for severe disease when making a decision regarding the individual's risk of participation. Any subject who has COVID-19 or other significant infection, or in the judgment of the Investigator, may be at a high risk of COVID-19 or its complications should not be randomized.

In aspects, a subject does not have any one of: active LN, active severe or unstable neuropsychiatric SLE. In aspects, a subject does not have a diagnosis of non-SLE vasculitis syndrome, mixed connective tissue disease, or rheumatic (overlap) syndrome. In aspects, a subject does not use immunosuppressants, biologics, and DMARDS within the protocol defined washout periods.

### Score of Activity and Damage in DLE (SADDLE)

In aspects, a method comprises determining a SADDLE score. SADDLE measures severity of activity (erythema, scale, induration) and damage (scarring/atrophy and dyspigmentation) attributable to DLE. Summed scores range between 0 and 195. In aspects, a method comprises determining a change from baseline in a SADDLE score. In aspects, a mean change in SADDLE score is determined. In aspects, a SADDLE score changes by at least about 3%, 5%, 10%, 15%, 20%, 30%, 40%, 50%, 80%, 100%, 150%, 200%, or 400%. In aspects, a SADDLE score changes by at least about 5, 10, 20, 30, 40, 50, 60, 70, 80, 90, 100, or 120 points.

### Discoid Lupus Erythematosus Classification Criteria (DLECC) Score

In aspects, a method comprises determining a change from baseline in a DLECC score. In aspects, a DLECC score changes by at least about 3%, 5%, 10%, 15%, 20%, 30%, 40%, 50%, 80%, 100%, 150%, 200%, or 400%.

### Severity Alopecia Tool (SALT)

In aspects, a method comprises determining the percent change from baseline in SALT score of a treated subject. In aspects, a subject has moderate-to-severe AA as assessed by a SALT score of ≥50 and ≤95 at screening and baseline (Day 1). The SALT score can be computed by measuring the percentage of hair loss in each of 4 areas of the scalp-vertex (40%), right profile (18%), left profile (18%), and posterior (24%) and adding the total to achieve a composite score. The SALT score can be determined at any time point post treatment or before treatment to document a baseline. In aspects, a SALT score is determined at week 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or week 30. In aspects, a SALT score is reduced at weeks 12-20 and/or 28-36 in treated subjects as compared to untreated subjects.

In aspects, administration of a an ILT7 binding protein is effective in effectuating a percent change from baseline in SALT score at week 20-30, 22-26, 23-25, 24-28, or 23-26. In aspects, administration of a an ILT7 binding protein is effective in effectuating a percent change from baseline in SALT score at week 24. In aspects, administration of a an ILT7 binding protein is effective in stabilizing or reducing a percent change from baseline in SALT score as compared to baseline or an otherwise comparable subject lacking the administration of the ILT7 binding protein. In aspects, administration of a an ILT7 binding protein is effective in stabilizing or reducing a percent change from baseline in SALT score by at least about 5%, 10%, 20%, 30%, 40%, 50%, 70%, 90%, 100%, 120%, 140%, 160%, 180%, or up to about 200% as compared to baseline or an otherwise comparable subject lacking the administration of the ILT7 binding protein. In aspects, treated subjects achieve ≥50% reduction in SALT as compared to baseline.

In aspects, a method comprises determining a proportion of subjects with absolute SALT score of ≤5, 10, 15, 20, 25, 30, 35, 40, 45, 50, or 55 at weeks 30-40, 35-45, 40-48, 40-45, or 40-50. In aspects, a method comprises determining a proportion of subjects with absolute SALT score of ≤10, 20, 30, 50 at weeks 40-48.

### Clinician-Reported Outcome (ClinRO) for Eyebrow Hair Loss

A Clinician Reported Outcome (ClinRO) was used to grade eyebrow and eyelash involvement. The ClinRO for eyebrow hair Loss™ and the ClinRO for eyelash hair Loss™ are comprised of single item, 4-point Likert-type response scales to assess incremental severity of each ClinRO measure.

In aspects, the proportion of subjects achieving ClinRO for Eyebrow Hair Loss scores of 0 or 1 (full coverage or minimal gaps) with a ≥2-point improvement from baseline at each visit (among those with baseline scores ≥2 [significant gaps to no notable hair]) is determined. In aspects, the proportion of subjects achieving ClinRO for Eyelash Hair Loss scores of 0 or 1 (full coverage or minimal gaps) with a ≥2-point improvement from baseline at each visit (among those with baseline scores ≥2 [significant gaps to no notable hair]) is determined. In aspects, treatment with an ILT7 binding protein is effective in stabilizing or reducing eyelash and/or eyebrow loss.

In aspects, treated subjects maintain their ClinRO or achieve a reduced ClinRO for at least about 1 month, 3 months, 5 months, 8 months, 1 year, 1.5 years, or up to about 2 years post treatment with an ILT7 binding protein of the disclosure.

### Alopecia Density and Extent (ALODEX) Score

In aspects, a method comprises determining an ALODEX score. The ALODEX score combines both extent and hair density into an overall percentage of scalp hair loss. The density scale of 0 to 10 is related to percentage of terminal hair loss, where 100% hair loss is equal to 10, 90% is equal to 9, 80% is equal to 8, 70% is equal to 7, 60% is equal to 6, 50% is equal to 5, 40% is equal to 4, 30% is equal to 3, 20% is equal to 2, 10% is equal to 1 and no hair loss is equal to 0. The density assignments in each of the 1% scalp areas in a given quadrant are added together and divided by the maximum grade of hair loss (10) to give the percent hair loss for that quadrant. The score in each quadrant is then added together to give the ALODEX score. In aspects, treatment with an ILT7 binding protein of the disclosure is effective in maintaining or reducing an ALODEX score in a treated subject. In aspects, treatment with an ILT7 binding protein of the disclosure is effective in reducing an ALODEX score in a treated subject by at least about 1, 2, 3, 4, 5, 6, 7, 8, 9, or 10 points compared to an untreated subject. In aspects, treatment with an ILT7 binding protein of the disclosure is effective in reducing an ALODEX score in a treated subject by at least about 1-3, 2-4, 1-5, 3-4, 2-5, or 4-5 points compared to an untreated subject.

### Medical Photography

In aspects, medical photography is utilized in a method described herein. Medical photographs can be taken of any tissue of the subject. In aspects, a photograph of the full scalp is obtained. In aspects, photographs are taken of the eyebrows and/or eyelashes for subjects who have hair loss in these areas. Photographs can be taken at any time point. In aspects, photographs are taken before, during, and after treatment. In aspects, photographs are taken of areas selected from the group consisting of: scalp, eyebrows, and eyelashes. In aspects, photographs are taken of areas including but not limited to lesions (e.g., DLE lesions). Any suitable camera can be utilized for photography.

In aspects, a method comprises determining density of hair regrowth over time by way of medical photography. In aspects, digital photography is used to assess hair regrowth within a lesion over time. In aspects, medical photography is used to determine change in discoid lesion characteristics including but not limited to size and dyspigmentation over time.

### Pharmacokinetics

In aspects, a sample of a subject is obtained for pharmacokinetic analysis. Pharmacokinetics comprise absorption, distribution, and the two routes of drug elimination, metabolism, and excretion. Any sample of a subject can be utilized. In aspects, a sample comprises a blood or serum sample. Samples can be collected for analysis of ILT7 binding protein concentrations. Samples can be collected at any time. Samples can be collected before, during, and/or after administration of an ILT7 binding protein.

### Pharmacodynamics

In aspects, a sample of a subject is obtained for pharmacodynamic analysis. Blood or serum samples can be collected for PD analysis of ILT7 binding protein at the visits and timepoints specified herein. The PD analysis of ILT7 binding protein can include but may not be limited to the assessment of whole blood transcriptomics (e.g., IFN gene signature), pDC flow cytometry, PBMCs (ex. levels of other circulating cells), serum and plasma biomarkers, and/or blood MxA.

### Immunogenicity

In aspects, a sample of a subject is obtained. Suitable samples include but are not limited to blood, serum, PBMC, or cellular samples. In aspects, a sample are collected for analysis of serum ILT7 binding protein anti-drug antibodies (ADA). In aspects, if serum ADAs are not detected, treatment is continued. In aspects, if serum ILT7 binding protein ADAs are detected, treatment is continued. Suitable assays to detect ADAs are selected from the group consisting of: Enzyme-linked immunosobent assay (ELISA), Radioimmunoprecipitation assay (RIP), Electrochemiluminescence immunoassay (ECLIA), Surface plasmon resonance immunoassay (SPRIA), and combinations thereof.

### Vital Signs

In aspects, a method provided herein comprises obtaining vital signs of a subject. Vital signs are selected from the group consisting of: systolic blood pressure, diastolic blood pressure (mmHg), pulse (bpm), body temperature (C), respiratory rate (breaths/minute), weight (kg), and height (cm). In aspects, weight and height can be collected to calculate the BMI of a subject. Vital signs can be determined at any time for example before, during, and after treatment.

### Physical Examination

In aspects, a method comprises performing a physical examination of a subject. A physical examination comprises evaluating at least one of: general appearance, dermatological, head, eyes, ears, nose, throat, respiratory, cardiovascular, abdominal, neurological, musculoskeletal, and/or lymphatic.

A physical examination can be determined at any time for example before, during, and after treatment.

### Clinical Laboratory Tests

In aspects, a method comprises performing a laboratory test. A test can be selected from the group consisting of: urinalysis, hematology, chemistry panel (with or without liver function tests), and serum pregnancy test (screening) for women of childbearing potential. In aspects, a laboratory test evaluates levels of cell numbers, cytokines, chemokines, gene expression, and combinations thereof. In aspects, a laboratory test, evaluates levels of one or more of IFN-α, IL-17, and IFNγ. In aspects, a clinical laboratory test comprises determining a level of eGFR. In aspects, a clinical laboratory test comprises determining a level of UPCR. In aspects, a method comprises determining a proportion of subjects achieving CRR as determined by one or more of: eGFR≥60 mL/min/1.73 m², eGFR no worse than 15% below a baseline level, a 24-hour UPCR≤0.5 mg/mg, or combinations thereof. In aspects, a method comprises determining a proportion of subjects achieving ORR as determined by one or more of: eGFR≥60 mL/min/1.73 m², eGFR no worse than 15% below a baseline level, or improvement in 24-hour UPCR. Improvement in UPCR can be determined by a baseline UPCR≤3.0 mg/mg: <1.0 mg/mg, or for subjects with a baseline UPCR>3.0 mg/mg: >50% improvement from baseline and ≤3.0 mg/mg.

In aspects, a method comprises determining a level of IFN in a subject with an autoimmune disease and treating the subject with an ILT7 binding protein of the disclosure if the subject is IFN^{hi} as determined by an in vitro assay and as compared to an otherwise comparable healthy subject. In aspects, IFN^{hi} comprises a level of IFN that is at least about 5%, 10%, 15%, 20%, 25%, or 30% above an average level of a healthy subject pool or a healthy baseline level of a subject.

### Quality of Life

In aspects, a method comprises determining quality of life. In aspects, administration of a composition provided herein is effective in improving quality of life of a treated subject. Quality of life can be assessed with one or more of: Patient Global Assessment (PGA), Patient Global Impression of Change (PGIC) scales, Dermatology Life Quality Index (DLQI) questionnaire, Cutaneous Lupus Erythematosus Quality of Life (CLE-QOL) questionnaire, or the EQ-5D-5L questionnaire. In aspects, a score improves (increases or decreases) by at least about 1, 3, 5, 10, 20, 30, 50, 60, 80, or 100 points. In aspects, a score is reduced following treatment with a composition provided herein. In aspects, a score is reduced by at least 5%, 10%, 20%, 30%, or 40% following treatment.

### Pharmaceutical Compositions

The present disclosure is also directed to pharmaceutical compositions comprising the ILT7-binding proteins used in the methods described herein. In aspects, the present disclosure provides for the use of an ILT7-binding protein used in the methods described herein in the manufacture of a medicament for treating a subject.

In aspects, a composition may comprise from about 100 mg/mL to about 170 mg/mL of an anti-ILT7 binding protein. In aspects, a composition may comprise from about 110 mg/ml to about 150 mg/mL of an anti-ILT7 binding protein. In aspects, a composition may comprise from about 120 mg/mL to about 150 mg/mL of an anti-ILT7 binding protein. In aspects, a composition may comprise from about 140 mg/mL to about 160 mg/mL of an anti-ILT7 binding protein. In aspects, a composition may comprise from about 145 mg/mL to about 155 mg/mL of an anti-ILT7 binding protein.

In aspects, a composition may comprise from about, at least about, or at most about: 100 mg/mL, 102 mg/mL, 104 mg/mL, 106 mg/mL, 108 mg/mL, 110 mg/mL, 112 mg/mL, 114 mg/mL, 116 mg/mL, 118 mg/mL, 120 mg/mL, 122 mg/mL, 124 mg/mL, 126 mg/mL, 128 mg/mL, 130 mg/mL, 132 mg/mL, 134 mg/mL, 136 mg/mL, 138 mg/mL, 140 mg/mL, 142 mg/mL, 144 mg/mL, 146 mg/mL, 148 mg/mL, 150 mg/mL, 155 mg/mL, 160 mg/mL, or up to about 170 mg/mL of an anti-ILT7 binding protein.

In aspects, a therapeutically effective amount of an ILT7-binding protein used in the methods described herein ranges from about 0.1 mg to about 1000 mg. In other aspects, a therapeutically effective amount of an ILT7-binding protein used in the methods described herein ranges from about 50 mg to about 300 mg. In aspects, a therapeutically effective amount of an ILT7-binding protein used in the methods described herein is about 200 mg, about 225 mg, about 250 mg, about 275 mg, about 300 mg, about 325 mg, about 350 mg, about 375 mg, about 400 mg, about 450 mg, or about 500 mg in a single dose. In aspects, a therapeutically effective amount of an ILT7-binding protein used in the methods described herein is about 200 mg in a single dose. A therapeutically effective amount of an ILT7-binding protein used in the methods described herein may be administered to a subject in need thereof in a single dose or in multiple doses. In aspects, a therapeutically effective amount of an ILT7-binding protein used in the methods described herein ranges from about 0.1 mg to about 1000 mg. In aspects, a therapeutically effective amount of an ILT7-binding protein used in the methods described herein ranges from about 50 mg to about 300 mg. In aspects, a therapeutically effective amount of an ILT7-binding protein used in the methods described herein is about 150 mg, about 175 mg, about 200 mg, about 225 mg, about 250 mg, about 275 mg, about 300 mg, about 325 mg, about 350 mg, about 375 mg, about 400 mg, about 450 mg, or about 500 mg in a single dose. In aspects, a therapeutically effective amount of an ILT7-binding protein used in the methods described herein is about 300 mg in a single dose. In aspects, the ILT7-binding protein is administered at a dose of about 20-50 mg, 30-50 mg, 40-60 mg, 50-100 mg, 50-200 mg, 100-150 mg, 50-150 mg, 100-200 mg, 100-250 mg, 50-300 mg, 200-250 mg, 200-300 mg, 150 mg-300 mg, 150-350 mg, or 175-375 mg. A therapeutically effective amount of an ILT7-binding protein used in the methods described herein may be administered to a subject in need thereof in a single dose or in multiple doses.

In aspects, a therapeutically effective amount of the ILT7 binding protein comprises an administration of at least about 300 mg. In aspects, at least two administrations of the ILT7 binding protein are needed to achieve the therapeutically effective dose. In aspects, an administration comprises two subcutaneous injections of the ILT7 binding protein. The subcutaneous injections can be administered as two 1.5 mL injections of ILT7 binding protein. In aspects, a treatment regimen comprises administrations of ILT7 binding protein about once every 4 weeks.

The methods comprise administering to the subject a pharmaceutically effective amount of an ILT7-binding protein. In aspects, the ILT7-binding protein is administered at a dose of 100 mg, 150 mg, 200 mg, 250 mg, and/or 300 mg. In aspects, the ILT7-binding protein is administered once about every four weeks. In aspects, the ILT7-binding protein is administered once about every twelve weeks. In aspects, the ILT7-binding protein is administered once about every twelve weeks and the subject is administered an additional 300 mg dose of the ILT7-binding protein about four weeks after the initial dose.

In aspects, the disclosure is directed to a method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg. In aspects, the pharmaceutically effective amount is about 300 mg. In aspects, the autoimmune disorder is lupus. In aspects, the autoimmune disorder is SLE. In particular aspects, the ILT7-binding protein is administered once about every two weeks, once about every three weeks, once about every four weeks, once about every five weeks, once about every six weeks, once about every eight weeks, once about every ten weeks, once about every twelve weeks, about once a month, once about every two months, once about every three months, once about every four months or once about every six weeks. In aspects, the ILT7-binding protein is administered once about every four weeks. In aspects, the ILT7-binding protein is administered once about every twelve weeks. In aspects, the ILT7-binding protein is administered once about every twelve weeks, and the subject is administered an additional 200 mg dose of the ILT7-binding protein about two, three, or four weeks after the initial dose. In aspects, the ILT7-binding protein is administered once about every twelve weeks, and the subject is administered an additional 200 mg dose of the ILT7-binding protein about four weeks after the initial dose. In one aspect, the disclosure is directed to method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, and wherein the subject is administered: a first dose, a second dose about four weeks after the first dose, a third dose about twelve weeks after the first dose, and subsequent doses every twelve weeks after the third dose. In one aspect, the disclosure is directed to a method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, and wherein the ILT7-binding protein is administered once about every four weeks. In aspects, the ILT7-binding protein is administered once about every twelve weeks, and the subject is administered an additional 200 mg dose of the ILT7-binding protein about four weeks after the initial dose. In aspects, the ILT-7 binding protein is daxdilimab. In aspects, the autoimmune disease is lupus. In aspects, the autoimmune disease is SLE. In aspects, the autoimmune disease is DLE. In aspects, the autoimmune disease is dermatomyositis. In aspects, the autoimmune disease is alopecia areata.

In aspects, the autoimmune disease treated with the ILT7-binding protein is DLE. In aspects, ILT7-binding protein is administered at a dose of 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 325 mg or 350 mg. In embodiments, the ILT7-binding protein is administered one about every week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks. 16 weeks, 20 weeks, or about once a month, about every two months, about every three months, about every four months, about every six months. In aspects, the ILT7-binding protein is administered at a dose of about 120 mg-200 mg every 4 weeks, 50-300 mg every 4 weeks, or 150 mg-300 mg every 12 weeks/quarterly. In aspects, the ILT7-binding protein is administered at a dose of about 150 mg every 4 weeks, 50-300 mg every 4 weeks, or 150 mg-300 mg every 12 weeks/quarterly. In embodiments, one or more loading doses are initially administered prior to one or more maintenance doses. In aspects, the ILT-7 binding protein is Daxdilimab.

In aspects, the autoimmune disease treated with the ILT7-binding protein is SLE. In aspects, ILT7-binding protein is administered at a dose of 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 325 mg or 350 mg. In embodiments, the ILT7-binding protein is administered one about every week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks. 16 weeks, 20 weeks, or about once a month, about every two months, about every three months, about every four months, about every six months. In aspects, the ILT7-binding protein is administered at a dose of about 120 mg-200 mg every 4 weeks, 50-300 mg every 4 weeks, or 150 mg-300 mg every 12 weeks/quarterly. In aspects, the ILT7-binding protein is administered at a dose of about 150 mg every 4 weeks, 50-300 mg every 4 weeks, or 150 mg-300 mg every 12 weeks/quarterly. In embodiments, one or more loading doses are initially administered prior to one or more maintenance doses. In aspects, the ILT-7 binding protein is Daxdilimab.

In aspects, the ILT7-binding protein is administered once about every four weeks. In aspects, the ILT7-binding protein is administered once about every twelve weeks. In one aspect, the ILT7-binding protein is administered once about every twelve weeks, and the subject is administered an additional 200 mg dose of the ILT7-binding protein about four weeks after the initial dose. In aspects, the pharmaceutically effective amount of the ILT7-binding protein is about 200 mg, and wherein the subject is administered: a first dose, a second dose about four weeks after the first dose, a third dose about twelve weeks after the first dose, and subsequent doses every twelve weeks after the third dose. In one aspect, the ILT-7 binding protein is daxdilimab. In aspects, the autoimmune disease is lupus. In a particular aspect, the autoimmune disease is SLE.

In aspects, the autoimmune disease treated with the ILT7-binding protein is Dermatomyositis/Anti-Synthetase Inflammatory Myositis (DM/ASIM). In some aspects, ILT7-binding protein is administered at a dose of 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 325 mg or 350 mg. In embodiments, the ILT7-binding protein is administered one about every week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks. 16 weeks, 20 weeks, or about once a month, about every two months, about every three months, about every four months, about every six months. In aspects, the ILT7-binding protein is administered at a dose of about 120 mg-200 mg every 4 weeks, 50-300 mg every 4 weeks, or 150 mg-300 mg every 12 weeks/quarterly. In aspects, the ILT7-binding protein is administered at a dose of about 150 mg every 4 weeks, 50-300 mg every 4 weeks, or 150 mg-300 mg every 12 weeks/quarterly. In embodiments, one or more loading doses are initially administered prior to one or more maintenance doses. In aspects, the ILT-7 binding protein is Daxdilimab.

In aspects, the autoimmune disease treated with the ILT7-binding protein is lupus nephritis. In aspects, ILT7-binding protein is administered at a dose of 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 325 mg or 350 mg. In embodiments, the ILT7-binding protein is administered one about every week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks. 16 weeks, 20 weeks, or about once a month, about every two months, about every three months, about every four months, about every six months. In aspects, the ILT7-binding protein is administered at a dose of about 120 mg-200 mg every 4 weeks, 50-300 mg every 4 weeks, or 150 mg-300 mg every 12 weeks/quarterly. In aspects, the ILT7-binding protein is administered at a dose of about 150 mg every 4 weeks, 50-300 mg every 4 weeks, or 150 mg-300 mg every 12 weeks/quarterly. In embodiments, one or more loading doses are initially administered prior to one or more maintenance doses. In aspects, the ILT-7 binding protein is Daxdilimab. In aspects, LN is treated with 100 mg of Daxdilimab 100 mg at baseline, week 2, and week 4, followed by 100 mg every 4 weeks. In aspects, LN is treated with Daxdilimab at a dosage of 300 mg at baseline, week 2, and week 4, followed by 300 mg every four weeks. In aspects, LN is treated with Daxdilimab at a dosage of 100 mg or 300 mg every 12 weeks.

In aspects, the disclosure is directed to a method of reducing plasmacytoid dendritic cells (pDCs) in a tissue of a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 300 mg. In aspects, the ILT7-binding protein is administered once about every four weeks. In aspects, the disclosure is directed to a method of reducing plasmacytoid dendritic cells (pDCs) in a tissue of a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 300 mg, and wherein the subject is administered: a first dose, a second dose about four weeks after the first dose, and additional doses every four weeks for a total of nine doses. In aspects, the disclosure is directed to a method of reducing plasmacytoid dendritic cells (pDCs) in a tissue of a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 300 mg, and wherein the ILT7-binding protein is administered once about every four weeks. In aspects, the ILT-7 binding protein is Daxdilimab.

In aspects, the disclosure is directed to a method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 100 mg or 300 mg. In aspects, the ILT7-binding protein is administered once about every two weeks, once about every three weeks, once about every four weeks, once about every five weeks, once about every six weeks, once about every eight weeks, once about every ten weeks, once about every twelve weeks, about once a month, once about every two months, once about every three months, once about every four months or once about every six weeks. In aspects, the ILT7-binding protein is administered once about every four weeks. In aspects, the ILT7-binding protein is administered once about every twelve weeks. In aspects, the ILT7-binding protein is administered at a dose of about 150 mg every 4 weeks, about 50 to about 300 mg every 4 weeks, or about 150 mg to about 300 mg every 12 weeks/quarterly. In aspects, the ILT7-binding protein is administered once about every four weeks, and the subject is administered an additional 300 mg dose of the ILT7-binding protein about four weeks after the initial dose. In aspects, the ILT7-binding protein is administered once about every four weeks, and the subject is administered an additional 300 mg dose of the ILT7-binding protein about four weeks after the initial dose. In aspects, the disclosure is directed to method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 300 mg, and wherein the subject is administered: a first dose, a second dose about four weeks after the first dose, a third dose about twelve weeks after the first dose, and subsequent doses every twelve weeks after the third dose. In aspects, the disclosure is directed to a method of treating alopecia acreata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 300 mg, and wherein the ILT7-binding protein is administered once about every four weeks. In aspects, the ILT7-binding protein is administered once about every twelve weeks, and the subject is administered an additional 300 mg dose of the ILT7-binding protein about four weeks after the initial dose. In aspects, the ILT7-binding protein is administered at a dose of about 120 mg-200 mg every 4 weeks, 50-300 mg every 4 weeks, or 150 mg-300 mg every 12 weeks/quarterly. In aspects, the ILT7-binding protein is administered at a dose of about 150 mg every 4 weeks, about 50 mg to about 300 mg every 4 weeks, or about 150 mg to about 300 mg every 12 weeks/quarterly. In aspects, the ILT-7 binding protein is Daxdilimab.

In aspects, administration of a composition provided herein results in a reduction in the level of pDCs in a tissue of a treated subject as compared to the baseline value of the subject prior to the administration wherein the reduction is at least about 3%, 5%, 10%, 15%, 20%, 25%, 30%, 35%, 40%, 45%, 50%, 55%, 60%, 65%, 70%, 75%, 80%, 85%90%, 100%, 120%, 130%, 140%, 150%, 200%, 250%, 300%, 400%, or up to about 500%.

In aspects, the autoimmune disease treated with the ILT7-binding protein is alopecia areata. In aspects, ILT7-binding protein is administered at a dose of 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 325 mg or 350 mg. In embodiments, the ILT7-binding protein is administered one about every week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks. 16 weeks, 20 weeks, or about once a month, about every two months, about every three months, about every four months, about every six months. In aspects, the ILT7-binding protein is administered at a dose of about 120 mg-200 mg every 4 weeks, 50-300 mg every 4 weeks, and 150 mg-300 mg every 12 weeks/quarterly. In aspects, the ILT7-binding protein is administered at a dose of about 150 mg every 4 weeks, 50-300 mg every 4 weeks, and/or 150 mg-300 mg every 12 weeks/quarterly. In embodiments, one or more loading doses are initially administered prior to one or more maintenance doses. In aspects, the ILT-7 binding protein is Daxdilimab.

In aspects, the ILT7-binding protein is administered once about every four weeks. In aspects, the ILT7-binding protein is administered once about every twelve weeks. In aspects, the ILT7-binding protein is administered once about every twelve weeks, and the subject is administered an additional 300 mg dose of the ILT7-binding protein about four weeks after the initial dose. In aspects, the pharmaceutically effective amount of the ILT7-binding protein is about 300 mg, and wherein the subject is administered: a first dose, additional doses about every four weeks for a total of nine doses. In aspects, the ILT-7 binding protein is Daxdilimab.

In aspects, any of the provided compositions are administered with one or more standard-of-care (SOC) therapies or concomitant therapies. In aspects, a SOC therapy comprises a corticosteroid. Exemplary corticosteroids comprise dexamethasone, hydrocortisone, methylprednisolone, and prednisone. In aspects, a corticosteroid is prednisone. In aspects, a method comprises tapering of a SOC therapy. Suitable tapering schedules are provided herein. In aspects, a tapering schedule comprises reducing a corticosteroid to a prednisone-equivalent dose of ≤7.5 mg/day by week 12 and prednisone-equivalent dose of ≤2.5 mg/day by week 24. In aspects, a SOC therapy comprises an immunosuppressive. In aspects, an immunosuppressive comprises mycophenolic acid. In aspects, a method is effective in reducing or eliminating an autoimmune disease in a subject administered an ILT7 binding protein of the disclosure in combination with a SOC therapy as compared to an otherwise comparable subject only administered the SOC therapy.

In aspects, a pharmaceutical composition of the disclosure comprises an ILT7-binding protein disclosed herein and one or more pharmaceutically acceptable carriers, diluents, or excipients. In this regard, “pharmaceutically acceptable carriers, diluents, or excipients” include but are not limited to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier that may or may not have been approved by the United States Food and Drug Administration as being acceptable for use in humans or domestic animals. For example, appropriate carriers are known to those skilled in the art and include stabilizers, diluents, and buffers. Suitable stabilizers include carbohydrates, such as sorbitol, lactose, mannitol, starch, sucrose, dextran, and glucose, and proteins, such as albumin or casein. Suitable diluents include saline, Hanks Balanced Salts, and Ringer's solution. Suitable buffers include an alkali metal phosphate, an alkali metal carbonate, or an alkaline earth metal carbonate.

In aspects, the pharmaceutical compositions of the disclosure may further contain one or more auxiliary substance, such one or more lipids, phospholipids, carbohydrates, and lipopolysaccharides. In aspects, pharmaceutical compositions of the disclosure optionally comprise one or more additional active substances.

In aspects, the pharmaceutical compositions of the disclosure can be prepared by techniques known to those skilled in the art. General considerations in the formulation and/or manufacture of pharmaceutical compositions may be found, for example, in Remington: The Science and Practice of Pharmacy 21st ed., Lippincott Williams & Wilkins, 2005 (incorporated herein by reference in its entirety). Generally, an ILT7-binding protein used in the methods described herein or fragments thereof is mixed with a carrier to form a solution, suspension, or emulsion. One or more of the additives discussed herein may be added in the carrier or may be added subsequently. The pharmaceutical compositions of the disclosure may be an aqueous solution, emulsion or suspension or may be a dried preparation. In aspects, the pharmaceutical compositions of the disclosure may be desiccated or lyophilized, for example, by freeze drying or spray drying for storage or formulations purposes. They may be subsequently reconstituted into liquid compositions by the addition of an appropriate liquid carrier or administered in dry formulation using methods known to those skilled in the art. In aspects, the ILT7-binding proteins used in the methods described herein are stored as lyophilized powder and subsequently reconstituted into liquid compositions prior to administration into a subject in need thereof.

The choice of administration of the pharmaceutical composition will depend on the formulation that is selected. The pharmaceutical compositions of the disclosure are administered in a manner compatible with the dosage formulation, and in such amount as are therapeutically effective. In aspects, a pharmaceutical composition of the disclosure is formulated into preparations in solid, semi-solid, liquid or gaseous forms, including, but not limited to, tablets, capsules, powders, granules, ointments, solutions, suppositories, injections, inhalants, gels, microspheres, and aerosols.

In aspects, a pharmaceutical composition comprising an ILT7-binding protein used in the methods described herein may be in the form of a solid or liquid. In aspects, the carrier(s) are particulate so that the compositions are, for example, in tablet or powder form. In other aspects, the carrier(s) are liquid, with a composition being, for example, an oral syrup, injectable liquid or an aerosol, which is useful in, for example, inhalatory administration. When intended for oral administration, a pharmaceutical composition comprising an ILT7-binding protein used in the methods described herein is in either solid or liquid form, where semi-solid, semi-liquid, suspension and gel forms are included within the forms considered herein as either solid or liquid.

In aspects, as a solid composition for oral administration, a pharmaceutical composition comprising an ILT7-binding protein used in the methods described herein may be formulated into a powder, granule, compressed tablet, pill, capsule, chewing gum, wafer or the like form. In aspects, such a solid composition will typically contain one or more inert diluents or edible carriers. In aspects, one or more of the following may be additionally present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

In aspects, when a pharmaceutical composition of the disclosure is in the form of a capsule, for example, a gelatin capsule, it may contain, in addition to materials disclosed herein, a liquid carrier such as polyethylene glycol or oil. Oral formulations may also include normally employed excipients such as, for example, pharmaceutical grades of saccharine, cellulose and magnesium carbonate.

In aspects, a pharmaceutical composition of the disclosure is in the form of a liquid, for example, an elixir, syrup, solution, emulsion or suspension. In aspects, the liquid may be for oral administration or for delivery by injection. In aspects, when intended for oral administration, the pharmaceutical compositions of the disclosure contain, in addition to an ILT7-binding protein used in the methods described herein, one or more of a sweetening agent, preservatives, dye/colorant and flavor enhancer. In aspects, in a pharmaceutical composition intended to be administered by injection, one or more of a surfactant, preservative, wetting agent, dispersing agent, suspending agent, buffer, stabilizer and isotonic agent may be included. In aspects, a pharmaceutical composition of the disclosure is administered to a subject in need thereof intravenously. In aspects, a pharmaceutical composition of the disclosure is administered to a subject in need thereof by subcutaneous injection.

In aspects, liquid pharmaceutical compositions comprising an ILT7-binding protein used in the methods described herein, whether they be solutions, suspensions or other like form, may include one or more of the following components: sterile diluents such as water for injection, saline solution, e.g., physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In aspects, the preparation can be enclosed in ampoules, disposable syringes or multiple dose vials made of glass or plastic. In aspects, an injectable pharmaceutical composition is preferably sterile.

In other aspects, a pharmaceutical composition comprising an ILT7-binding protein used in the methods described herein may be intended for topical administration, in which case the carrier may suitably comprise a solution, emulsion, ointment or gel base. In aspects, the base, for example, may comprise one or more of the following: petrolatum, lanolin, polyethylene glycols, bee wax, mineral oil, diluents such as water and alcohol, and emulsifiers and stabilizers. In other aspects, thickening agents may be present in a pharmaceutical composition for topical administration. In aspects, if intended for transdermal administration, a pharmaceutical composition of an ILT7-binding protein used in the methods described herein may be included with a transdermal patch or iontophoresis device.

In aspects, the pharmaceutical composition comprising an ILT7-binding protein used in the methods described herein is intended for rectal administration, in the form, for example, of a suppository. For suppositories, binders and carriers may include, for example, polyalkalene glycols or triglycerides. In aspects, a composition for rectal administration contains an oleaginous base as a suitable nonirritating excipient. Such bases include, without limitation, lanolin, cocoa butter or polyethylene glycol.

In aspects, a pharmaceutical composition comprising an ILT7-binding protein used in the methods described herein comprises dosage units that can be administered as an aerosol. The term aerosol is used to denote a variety of systems ranging from those of colloidal nature to systems consisting of pressurized packages. In aspects, delivery is accomplished by a liquefied or compressed gas or by a suitable pump system that dispenses the active ingredients. In aspects, aerosols of an ILT7-binding protein used in the methods described herein may be delivered in single phase, bi-phasic, or tri-phasic systems in order to deliver the active ingredient(s). In other aspects, delivery of the aerosol includes the necessary container, activators, valves, sub containers, and the like, which together may form a kit. One skilled in the art can readily determine specific aerosol formulations and delivery modes.

Pharmaceutical compositions of the disclosure may be administered in a suitable, nontoxic pharmaceutical carrier, may be comprised in microcapsules, microbeads, and/or may be comprised in a sustained release implant.

In aspects, pharmaceutical compositions of the disclosure include materials that form a coating shell around the active ingredients. In aspects, the materials that form the coating shell are typically inert, and may be selected from, for example, sugar, shellac, and other enteric coating agents.

In aspects, the pharmaceutical compositions of the disclosure in solid or liquid form include an agent that binds to an ILT7-binding protein used in the methods described herein and thereby assist in the delivery of the ILT7-binding protein used in the methods described herein. In aspects, suitable agents that act in this capacity include a protein or a liposome.

In aspects, pharmaceutical compositions that are administered to a subject take the form of one or more dosage units, where, for example, a tablet may be a single dosage unit, and a container of an ILT7-binding protein used in the methods described herein in aerosol form may hold a plurality of dosage units. Actual methods of preparing such dosage forms are known, or are apparent to those skilled in this art; for example, see Remington: The Science and Practice of Pharmacy, 20th Edition (Philadelphia College of Pharmacy and Science, 2000). A composition to be administered will, in any event, contain a therapeutically effective amount of an ILT7-binding protein used in the methods described herein, or a pharmaceutically acceptable salt thereof, to aid in treatment of a disease or condition of interest in accordance with the teachings herein.

In aspects, the pharmaceutical compositions of the disclosure comprise one or more additional therapeutically active substances. In other aspects, a therapeutically effective dose of the pharmaceutical compositions of the disclosure is administered to a subject in need thereof in combination with one or more additional therapeutically active substances. As used herein, a “combination” refers to a combination comprising an ILT7-binding protein used in the methods described herein and one or more additional therapeutically active substances, each of which may be administered serially (sequentially), concurrently or simultaneously.

Pharmaceutical compositions of the disclosure may desirably be administered at several intervals in order to sustain therapeutic levels. Pharmaceutical compositions of the disclosure may be used in conjunction with other bactericidal or bacteriostatic methods.

Although the descriptions of pharmaceutical compositions provided herein are principally directed to pharmaceutical compositions which are suitable for administration to humans, it are understood by the skilled artisan that such compositions are generally suitable for administration to subjects of all sorts. In aspects, the subject is a mammal. In aspects, a mammal includes primates, such as humans, monkeys and apes, and non-primates such as domestic animals, including laboratory animals and household pets and farm animals (e.g., cats, dogs, swine, cattle, sheep, goats, horses, rabbits), and non-domestic animals, such as wildlife, birds, or the like.

### Kits

Provided are also kits comprising an anti-ILT7 binding protein. In aspects, a kit comprises a) a pharmaceutical composition comprising an anti-ILT7-binding protein for use in the treatment of alopecia in a subject in need thereof; and b) instructions describing how to administer said pharmaceutical composition to a subject in need thereof.

Kits may comprise an anti-ILT7 binding protein (e.g., in liquid or lyophilized form) or a pharmaceutical composition comprising the anti-ILT7 binding protein. Additionally, such kits may comprise means for administering the anti-ILT7 binding protein (e.g., a syringe or a prefilled pen) and instructions for use. These kits may contain additional therapeutic agents.

In aspects, a container is comprised within a kit and can be a vial. A vial can contain a lyophilized dosage form of a composition provided herein. In embodiments, a vial can contain a liquid dosage form of a composition provided herein. In embodiments, a container is a vial and contains about 1 mL, 1.25 mLs, 1.5 mLs, 1.75 mLs, 2 mLs, 2.25 mLs, 2.5 mLs, 2.75 mLs, 3 mLs, 3.25 mLs, 3.5 mLs, 3.75 mLs, 4 mLs, 4.25 mLs, 4.5 mLs, 4.75 mLs, 5 mLs, 5.25 mLs, 5.5 mLs, 5.75 mLs, 6 mLs, 6.25 mLs, 6.5 mLs, 6.75 mLs, 7 mLs, 7.25 mLs, 7.5 mLs, 7.75 mLs, 8 mLs, 8.25 mLs, 8.5 mLs, 8.75 mLs, 9 mLs, 9.25 mLs, 9.5 mLs, 9.75 mLs, 10 mLs of solution.

### NUMBERED EMBODIMENTS

Notwithstanding the appended claims, the disclosure sets forth the following numbered embodiments:

### Embodiment Set 1

1. A method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg.

2. A method of reducing plasmacytoid dendritic cells (pDCs) in a tissue of a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg.

3. A method for reducing a type I interferon gene signature (IFNGS) in a subject in need thereof, the method comprising administering to the subject a pharmaceutically effective amount of an immunoglobulin-like transcript 7 (ILT7)-binding protein, wherein the ILT7 binding protein is administered to the subject when the type I IFNGS is elevated in the subject relative to the type I IFNGS in a normal subject, wherein the pharmaceutically effective amount of the ILT7-binding protein is about 200 mg.

4. The method of embodiment 3, wherein the type I IFNGS is measured in a test biological sample taken from the subject wherein the test sample is selected from the group consisting of blood, sputum, saliva, skin cells, skin biopsy samples, kidney cells, lung cells, liver cells, heart cells, brain cells, nervous tissue, thyroid cells, eye cells, skeletal muscle cells, cartilage, bone tissue, and cultured cells.

5. The method of embodiment 4, wherein the test biological sample is blood, skin cells or skin biopsy samples.

6. The method according to any one of the preceding embodiments, wherein the type I IFNGS is elevated by at least about 4-fold in the test biological sample relative to an otherwise comparable normal biological sample.

7. The method according to any one of the preceding embodiments, wherein the type I IFNGS comprises the collective expression levels of two or more type I interferon (IFN)-inducible genes.

8. The method of embodiment 7, wherein the two or more type I interferon (IFN)-inducible genes are selected from the group consisting of SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18.

9. The method according to embodiment 7, wherein the type I IFNGS comprises the collective expression levels of all of SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18.

10. The method of embodiment 7, wherein the type I IFNGS is determined by assaying mRNA levels of the two or more type I interferon (IFN)-inducible genes in the test biological sample.

11. The method of embodiment 8, wherein the type I IFNGS is determined by assaying mRNA levels of: SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18 in the test biological sample.

12. The method according to any one of the preceding embodiments, wherein the administering of the ILT7-binding protein reduces plasmacytoid dendritic cells (pDCs) in the subject.

13. The method of embodiment 12, wherein the pDCs are circulating pDCs.

14. The method of any one of embodiments 12 or 13, wherein the reduction in the pDCs is reversible.

15. The method according to any one of the preceding embodiments, wherein reducing the type I IFNGS treats an autoimmune disease in the subject.

16. The method according to any one of the preceding embodiments, wherein the ILT7-binding protein is administered once about every four weeks.

17. The method of any one of embodiments 1-15, wherein the ILT7-binding protein is administered once about every twelve weeks.

18. The method of embodiment 17, wherein the subject is administered an additional 200 mg dose of the ILT7-binding protein about four weeks after an initial dose.

19. A method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, and wherein the subject is administered: a) a first dose, b) a second dose about four weeks after the first dose, c) a third dose about twelve weeks after the first dose, and d) subsequent doses about twelve weeks after the third dose.

20. A method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, and wherein the ILT7-binding protein is administered once about every four weeks.

21. A method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, wherein the ILT-7 binding protein is administered once about every twelve weeks and the subject is administered an additional 200 mg dose of the ILT7-binding protein about four weeks after an initial dose.

22. A method of reducing plasmacytoid dendritic cells (pDCs) in a tissue of a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, and wherein the subject is administered: a) a first dose, b) a second dose about four weeks after the first dose, c) a third dose about twelve weeks after the first dose, and d) subsequent doses about twelve weeks after the third dose.

23. A method of reducing plasmacytoid dendritic cells (pDCs) in a tissue of a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, and wherein the ILT7-binding protein is administered once about every four weeks.

24. A method of reducing plasmacytoid dendritic cells (pDCs) in a tissue of a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, wherein the ILT-7 binding protein is administered once about every twelve weeks and the subject is administered an additional 200 mg dose of the ILT7-binding protein about four weeks after an initial dose.

25. The method of any one of embodiments 22-24, wherein the decrease in pDCs in the tissue compared to the baseline value ranges from about 1% to about 99%.

26. The method of any one of embodiments 22-25, wherein the decrease in pDCs in the tissue compared to the baseline value is at least about 50%.

27. The method of any one of embodiments 1-26, wherein the subject has a high blood type I IFNGS level prior to administration of the ILT7-binding protein.

28. The method of any one of embodiments 1-27, wherein the subject has a high pDC level in a tissue biopsy prior to administration of the ILT7-binding protein.

29. The method of any one of embodiments 1-28, where the subject comprises an autoimmune disease, and wherein the autoimmune disease is systemic lupus erythematosus (SLE), lupus nephritis, cutaneous lupus erythematosus (CLE), Sjögren's syndrome, inflammatory myositis, such as dermatomyositis, inclusion body myositis, juvenile myositis and polymyositis, systemic sclerosis, diabetes, Hashimoto's disease, autoimmune adrenal insufficiency, pure red cell anemia, multiple sclerosis, rheumatic carditis, psoriasis, psoriatic arthritis, rheumatoid arthritis, chronic inflammation, chronic rheumatism, vitiligo, alopecia areata, hidradenitis suppurativa, celiac disease, acute and chronic graft versus host disease (GVHD), vascular inflammation, myocardial infarction, Type-1 interferonopathies, and combinations thereof.

30. The method of embodiment 29, wherein the autoimmune disease is SLE.

31. The method of embodiment 29, wherein the autoimmune disease is CLE.

32. The method of embodiment 29, wherein the autoimmune disease is lupus.

33. The method of any one of embodiments 1-32, wherein the subject does not have discoid lupus erythematosus (DLE).

34. The method of any one of embodiments 1-32, wherein the subject has DLE.

35. The method according to any one of the preceding embodiments, wherein the ILT7-binding protein is administered by subcutaneous injection.

36. The method according to any one of the preceding embodiments, wherein administration of the ILT7-binding protein leads to at least about 50% reduction in the type I IFNGS in the subject, compared to the type I IFNGS prior to administration of the ILT7-binding protein.

37. The method according to any one of the preceding embodiments, wherein the ILT7-binding protein induces antibody-dependent cell-mediated cytotoxicity (ADCC) activity against pDCs.

38. The method according to any one of embodiments 1-37, wherein the ILT7-binding protein at least suppresses release of type I interferon (IFN) from pDCs.

39. The method of embodiment 38, wherein the type I IFN is IFNα.

40. The method according to any one of the preceding embodiments, wherein the ILT7-binding protein binds to ILT7.

41. The method according to any one of the preceding embodiments, wherein the subject is determined to have a high blood type I IFNGS level prior to administration of the ILT7-binding protein.

42. The method according to any one of the preceding embodiments, wherein the ILT7-binding protein is an antibody comprising heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOs: 3, 4, 5, 6, 7, and 8, respectively.

43. The method according to any one of the preceding embodiments, wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2.

44. The method according to any one of the preceding embodiments, wherein the ILT7-binding protein is an antibody comprising a heavy chain variable region (VH) of SEQ ID NO:1 and a light chain variable region (VL) of SEQ ID NO:2.

45. The method according to any one of the preceding embodiments, wherein the ILT7 binding protein is afucosylated.

46. A method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2, and wherein the subject is administered: a) a first dose, b) a second dose about four weeks after the first dose, c) a third dose about twelve weeks after the first dose, and d) subsequent doses about twelve weeks after the third dose.

47. A method of treating Systemic Lupus Erythematosus in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, wherein the ILT7 binding protein is an antibody comprising a variable heavy chain (VH) that is at least 85%, 90%, 95%, 96%, 97%, 98% or 99% identical to SEQ ID NO: 1 and/or a variable light chain (VL) that is at least 85%, 90%, 95%, 96%, 97%, 98% or 99% identical to SEQ ID NO:2, and wherein the subject is administered: a) a first dose, b) a second dose about four weeks after the first dose, c) a third dose about twelve weeks after the first dose, and d) subsequent doses about twelve weeks after the third dose.

48. A method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2, and wherein the ILT7-binding protein is administered once about every four weeks.

49. A method of treating systemic lupus erythematosus in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2, and wherein the ILT7-binding protein is administered once about every four weeks.

50. A method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2, and wherein the ILT-7 binding protein is administered once about every twelve weeks, and the subject is administered an additional 200 mg dose of the ILT7-binding protein about four weeks after an initial dose.

51. A method of treating systemic lupus erythematosus in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 200 mg, wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2, and wherein the ILT-7 binding protein is administered once about every twelve weeks and the subject is administered an additional 200 mg dose of the ILT7-binding protein about four weeks after an initial dose.

52. A method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 150-350 mg, and wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2.

53. The method of any one of embodiments 1-52, wherein the ILT7-binding protein is administered with one or more additional therapies.

54. The method of embodiment 53, wherein one of the one or more additional therapies is a standard of care therapy.

55. The method of any one of embodiments 1-54, wherein administration of the ILT7-binding protein reduces occurrence of disease flares in the subject.

56. The method of embodiment 53, wherein the one or more additional therapies comprises a glucocorticoid.

57. The method of any one of embodiments 53-56, wherein the administration of the one or more additional therapies is tapered.

58. The method of any one of embodiments 53-57, wherein the autoimmune disorder is selected from the group consisting of: discoid lupus erythematosus (DLE), systemic lupus erythematosus (SLE), lupus nephritis, Dermatomyositis/Anti-Synthetase Inflammatory Myositis (DM/ASIM), and alopecia areata.

59. The method of embodiment 58, wherein the autoimmune disorder is DLE, and wherein the pharmaceutically effective amount of the ILT7-binding protein is about 300 mg.

60. The method of embodiment 58, wherein the autoimmune disorder is SLE, and wherein the pharmaceutically effective amount of the ILT7-binding protein is about 200 mg.

61. The method of embodiment 58, wherein the autoimmune disorder is lupus nephritis, and wherein the pharmaceutically effective amount of the ILT7-binding protein is about 300 mg.

62. A method of treating discoid lupus erythematosus (DLE) in a subject in need thereof, the method comprising administering to the subject a pharmaceutically effective amount of an immunoglobulin-like transcript 7 (ILT7)-binding protein

63. The method of embodiment 62, wherein the DLE is primary DLE.

64. The method of embodiment 62 or 63, wherein the subject does not have systemic lupus.

65. The method of any of embodiments 62-64, wherein the DLE is refractory.

66. The method of embodiment 65, wherein the DLE is refractory to one or more therapies selected from the group consisting of: a topical steroid, an intralesional steroids, an antimalarial, tacrolimus, thalidomide, optical tacrolimus, azathioprine, cyclosporin, mycophenolate mofetil, methotrexate, and acitretin.

67. The method of any of embodiments 62-66, wherein the ILT7 binding protein is an antibody.

68. The method of embodiment 67, wherein the antibody comprises heavy chain Complementarity-Determining Regions (HCDRs), HCDR1, HDR2, HCDR3, and light chain Complementarity Determining Regions (LCDRs), LCDR1, LCDR2, and LCDR3 having the amino acid sequences of SEQ ID NOs: 3, 4, 5, 6, 7, and 8.

69. The method of embodiment 67 or 68, wherein the antibody comprises a variable heavy chain (VH) that is at least 85%, 90%, 95%, 96%, 97%, 98%, 99% or 100% identical to SEQ ID NO:1 and a variable light chain (VL) that is at least 85%, 90%, 95%, 96%, 97%, 98%, 99% or 100% identical to SEQ ID NO:2.

70. A method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 250 mg to 350 mg.

71. A method of reducing plasmacytoid dendritic cells (pDCs) in a tissue of a subject with alopecia areata, the method comprising administering to the subject a pharmaceutically effective amount of an immunoglobulin-like transcript 7 (ILT7)-binding protein, wherein the pharmaceutically effective amount of the ILT7-binding protein is about 250 mg to 350 mg.

72. The method according to any one of the preceding embodiments, wherein the administering of the ILT7-binding protein reduces plasmacytoid dendritic cells (pDCs) in the subject.

73. The method of any one of embodiments 71-72, wherein the pDCs are circulating pDCs.

74. The method of any one of embodiments 72-73, wherein the reduction in the pDCs is reversible.

75. The method according to any one of embodiments 70-74, wherein the ILT7-binding protein is administered once about every four weeks.

76. A method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 250 mg to 350 mg, and wherein the ILT7-binding protein is administered once about every four weeks.

77. A method of reducing plasmacytoid dendritic cells (pDCs) in a tissue of a subject with alopecia areata, the method comprising administering to the subject a pharmaceutically effective amount of an immunoglobulin-like transcript 7 (ILT7)-binding protein, wherein the pharmaceutically effective amount of the ILT7-binding protein is about 250 mg to 350 mg, and wherein the ILT7-binding protein is administered once about every four weeks.

78. The method of any one of embodiments 71-77, wherein the reduction in pDCs in the tissue compared to the baseline value ranges from about 1% to about 99%.

79. The method of any one of embodiments 71-78, wherein the decrease in pDCs in the tissue compared to the baseline value is at least about 50%.

80. The method according to any one of the preceding embodiments, wherein the ILT7-binding protein induces antibody-dependent cell-mediated cytotoxicity (ADCC) activity against pDCs.

81. The method according to any one of embodiments 71-80, wherein the ILT7-binding protein at least suppresses release of type I interferon (IFN) from pDCs.

82. The method of embodiment 81, wherein the type I IFN is IFNα.

83. The method according to any one of embodiments 70-82, wherein the ILT7-binding protein binds to ILT7.

84. The method according to any one of embodiments 70-83, wherein the ILT7-binding protein is an antibody comprising heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOs: 3, 4, 5, 6, 7, and 8, respectively.

85. The method according to any one of embodiments 70-84, wherein the ILT7 binding protein is an antibody comprising a variable heavy chain (VH) that is at least 85%, 90%, 95%, 96%, 97%, 98% or 99% identical to SEQ ID NO: 1 and/or a variable light chain (VL) that is at least 85%, 90%, 95%, 96%, 97%, 98% or 99% identical to SEQ ID NO:2.

86. The method according to any one of embodiments 70-85, wherein the ILT7-binding protein is an antibody comprising a heavy chain variable region (VH) of SEQ ID NO:1 and a light chain variable region (VL) of SEQ ID NO:2.

87. The method according to any one of embodiments 70-86, wherein the ILT7 binding protein is afucosylated.

88. A method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 250 mg to 350 mg, wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2, and wherein the ILT7-binding protein is administered once about every four weeks.

89. A method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 250 mg-350 mg, and wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2.

90. The method of any one of embodiments 70-89, wherein the ILT7-binding protein is administered with one or more additional therapies.

91. The method of embodiment 90, wherein one of the one or more additional therapies is a standard of care therapy.

92. The method of any one of embodiments 70-91, wherein the ILT7-binding protein is administered subcutaneously.

93. The method according to any one of embodiments 70-92, wherein the administering is effective in stabilizing or reducing hair loss in the subject as determined by a stable or reduced Severity of Alopecia Tool (SALT) score and/or Alopecia Density and Extent (ALODEX) score.

94. The method according to any one of embodiments 70-93, wherein when the ILT7-binding protein is administered to a mouse with alopecia acreata, the administering is effective in reducing a level of type 1 interferon-inducible myxovirus protein A (MxA) in a biopsy comprising a hair follicle of the mouse as determined by immunohistochemistry.

95. The method according to any one of embodiments 70-94, wherein the pharmaceutically effective amount of the ILT7-binding protein is about 300 mg.

96. The method of embodiment 95, wherein the 300 mg is administered as two doses of 150 mg each.

97. The method according to any one of embodiments 70-96, wherein the ILT7 binding protein is Daxdilimab.

98. A method of treating lupus nephritis in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 300 mg, and wherein the ILT7-binding protein is administered once about every four weeks.

99. A method of treating lupus nephritis in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 100 mg, and wherein the ILT7-binding protein is administered once about every four weeks.

100. The method of any one or embodiments 98 or 99, wherein prior to receiving the ILT7-binding protein every four weeks, the subject is administered one or more initial doses of the ILT7-binding protein.

101. The method of embodiment 100, wherein the initial doses of the ILT7-binding protein are administered once about every two weeks.

102. The method of any one of embodiments 98-101, wherein the subject is assigned to a maintenance therapy after receiving at least four, five, six, seven, eight, nine, ten, eleven, twelve or more doses of the ILT-7 protein every four weeks.

103. The method of embodiments 102, wherein the maintenance dose is administered once about every twelve weeks.

104. The method of any one of embodiments 98-103, wherein the ILT7-binding protein is administered with one or more additional therapies.

105. The method of embodiment 104, wherein one of the one or more additional therapies is a corticosteroid.

106. The method of embodiment 105, wherein administration of the corticosteroid is tapered.

107. A method of treating DLE in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 300 mg, and wherein the ILT7-binding protein is administered once about every four weeks.

108. A method of treating DLE in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 150 mg, and wherein the ILT7-binding protein is administered once about every four weeks.

109. The method of any one of embodiments 107-108, wherein the administering is effective in reducing one or more of: Cutaneous Lupus Erythematosus Disease Area and Severity Index-Activity (CLASI-A) score, Cutaneous Lupus Erythematosus Disease Area and Severity Index-Damage (CLASI-D) score, Cutaneous Lupus Activity Investigator's Global Assessment (CLA-IGA) scale, Discoid Lupus Erythematosus Classification Criteria (DLECC) score, Score of Activity and Damage in Discoid Lupus Erythematosus (SADDLE), onset of new discoid lesions, size of lesions, or dyspigmentation of a discoid lesion.

### Embodiment Set 2

1. A method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is from about 100-350 mg.

2. The method of embodiment 1, wherein the autoimmune disease is selected from the group consisting of discoid lupus erythematosus (DLE), systemic lupus erythematosus (SLE), lupus nephritis, Dermatomyositis, Anti-Synthetase Inflammatory Myositis, and alopecia areata.

3. The method of embodiment 2, wherein the autoimmune disease is SLE, and wherein the pharmaceutically effective amount is about 200 mg.

4. The method of any one of the preceding embodiments, wherein the administering is effective in reducing: a) a level of plasmacytoid dendritic cells (pDCs) in a tissue of the subject; b) a type I interferon gene signature (IFNGS); or c) the level of plasmacytoid dendritic cells (pDCs) in the tissue of the subject and the type I IFNGS, each of each is as compared to a baseline level of the subject before the administering.

5. The method of embodiment 4, wherein the IFNGS comprises the collective expression levels of SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18.

6. The method of embodiment 4, wherein the reduction in the level of pDCs in the tissue compared to the baseline value is at least about 10%, 20%, 25%, 30%, 40%, 50%, 60%, or 70%.

7. The method of embodiment 2, wherein the autoimmune disease is lupus nephritis, and wherein the pharmaceutically effective amount is about 300 mg.

8. The method of embodiment 7, wherein after the administering, the subject achieves a positive renal response as determined by one or more of an improvement in a Glomerular Filtration Rate (eGFR) or 24-hour Urine Protein to Creatinine Ratio (UPCR) as compared to a baseline level of the subject before the administering.

9. The method of embodiment 2, wherein the autoimmune disease is discoid lupus erythematosus (DLE), and wherein the pharmaceutically effective amount is about 150-300 mg.

10. The method of embodiment 9, wherein the administering is effective in reducing one or more of: Cutaneous Lupus Erythematosus Disease Area and Severity Index-Activity (CLASI-A) score, Cutaneous Lupus Erythematosus Disease Area and Severity Index-Damage (CLASI-D) score, Cutaneous Lupus Activity Investigator's Global Assessment (CLA-IGA) scale, Discoid Lupus Erythematosus Classification Criteria (DLECC) score, Score of Activity and Damage in Discoid Lupus Erythematosus (SADDLE), onset of new discoid lesions, size of lesions, or dyspigmentation of a discoid lesion, as compared to a baseline level of the subject before the administering.

11. The method of embodiment 2, wherein the autoimmune disease is alopecia areata, and wherein the pharmaceutically effective amount is about 300 mg.

12. The method of embodiment 11, wherein the administering is effective in stabilizing or reducing hair loss in the subject as determined by a stable or reduced Severity of Alopecia Tool (SALT) score and/or Alopecia Density and Extent (ALODEX) score.

13. The method of any one of the preceding embodiments, wherein the ILT7-binding protein is administered with one or more additional therapies.

14. The method of embodiment 13, wherein the one or more additional therapies comprises a corticosteroid.

15. The method of embodiment 14, wherein the corticosteroid is prednisone.

16. The method of any one of embodiments 13-15, wherein the administration of the one or more additional therapies is tapered.

17. The method of embodiment 1, wherein the pharmaceutically effective amount of the ILT7-binding protein is from about 150-300 mg.

18. The method of embodiment 17, wherein the pharmaceutically effective amount of the ILT7-binding protein is from about 200-300 mg.

19. The method of any one of the preceding embodiments, wherein the ILT7-binding protein is administered once about every four weeks or once about every twelve weeks.

20. The method of any one of the preceding embodiments, wherein the pharmaceutically effective amount of the ILT7-binding protein is about 300 mg and the 300 mg is administered as two doses of 150 mg each.

21. A method of treating discoid lupus erythematosus (DLE) in a subject in need thereof, the method comprising administering to the subject a pharmaceutically effective amount of an immunoglobulin-like transcript 7 (ILT7)-binding protein, wherein the pharmaceutically effective amount of the ILT7-binding protein is from about 100 to about 300 mg.

22. A method of treating systemic lupus erythematosus (SLE) in a subject in need thereof, the method comprising administering to the subject a pharmaceutically effective amount of an immunoglobulin-like transcript 7 (ILT7)-binding protein, wherein the pharmaceutically effective amount of the ILT7-binding protein is from about 200 mg.

23. A method of treating lupus nephritis in a subject in need thereof, the method comprising administering to the subject a pharmaceutically effective amount of an immunoglobulin-like transcript 7 (ILT7)-binding protein, wherein the pharmaceutically effective amount of the ILT7-binding protein is from about 300 mg.

24. A method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is from about 300 mg.

25. A method of treating dermatomyositis in a subject in need thereof, the method comprising administering to the subject a pharmaceutically effective amount of an immunoglobulin-like transcript 7 (ILT7)-binding protein.

26. A method of treating Anti-Synthetase Inflammatory Myositis in a subject in need thereof, the method comprising administering to the subject a pharmaceutically effective amount of an immunoglobulin-like transcript 7 (ILT7)-binding protein.

27. The method of any one of embodiments 25-26, wherein the pharmaceutically effective amount of the ILT7-binding protein is from about 100 mg to about 300 mg.

28. The method of any one of the preceding embodiments, wherein the ILT7 binding protein is an antibody that comprises heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOs: 3, 4, 5, 6, 7, and 8, respectively.

29. The method of embodiment 28, wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2.

30. The method of embodiment 29, wherein the ILT7-binding protein is an antibody comprising a heavy chain variable region (VH) of SEQ ID NO:1 and a light chain variable region (VL) of SEQ ID NO:2.

31. The method of any one of the preceding embodiments, wherein the ILT7 binding protein is afucosylated.

32. The method of any one of the preceding embodiments, wherein the ILT7 binding protein is Daxdilimab.

33. The method of any one of the preceding embodiments, wherein the administration is subcutaneous.

34. The method of any one of embodiments 21-33, wherein the subject is administered the ILT7-binding protein every 4 weeks.

35. The method of any one of embodiments 21-33, wherein the subject is administered the ILT7-binding protein every 12 weeks.

36. The method of any one of embodiments 21-35, wherein prior to the administering, the subject is administered at least one initial dose of the ILT7-binding protein.

37. The method of embodiment 36, wherein the at least one initial dose is administered every 2 weeks for 1, 2, 3, 4, 5, or more doses.

38. The method of any one of embodiments 36-37, wherein the at least one initial dose is about 100-300 mg.

39. A method of treating lupus nephritis in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 100 mg every 2 weeks for up to 4 weeks followed by 100 mg every 4 weeks for 1, 2, 3, 4, 5, 6, 7, 8, 9, 10 11, 12, 13, 14, 15 or more doses.

40. The method of embodiment 39, wherein the subject is further administered: 100 mg of the ILT7 binding protein every 12 weeks; or 300 mg of the ILT7 binding protein every 12 weeks following the every 4 weeks administration.

41. The method of embodiment 40, wherein the 100 mg every 12 weeks or the 300 mg every 12 weeks are continued for at least about 104 weeks.

42. A method of treating lupus nephritis in a subject in need thereof, the method comprising administering to the subject a pharmaceutically effective amount of immunoglobulin-like transcript 7 (ILT7)-binding protein, wherein the pharmaceutically effective amount of the ILT7-binding protein is about 300 mg every 2 weeks for up to 4 weeks followed by 300 mg every 12 weeks for 1, 2, 3, 4, 5, 6, 7, 8, 9, 10 11, 12, 13, 14, 15 or more doses.

### EXAMPLES

### Example 1: A Phase 2 Randomized, Double-Blind, Placebo-Controlled Efficacy and Safety Study of VIB7734 (Daxdilimab) for the Treatment of Moderate to Severely Active Systemic Lupus Erythematosus

A Phase 2, multicenter, international, double-blind, randomized, placebo-controlled, parallel-arm trial are carried out to assess the efficacy and safety of daxdilimab in subjects with moderate to severely active systemic lupus erythematosus (SLE).

The target population is adults aged ≥18 to ≤70 years who have moderate to severely active (recent flares or chronic active disease) SLE as defined by the SLE Disease Activity Index 2000 (SLEDAI-2K), British Isles Lupus Assessment Group (BILAG) 2004 Index, and Physician Global Assessment (PGA). In this study, approximately 195 subjects are randomized in a ratio of 1:1:1 (65 subjects per arm) to receive daxdilimab 200 mg Q4W SC, daxdilimab 200 mg every 12 weeks (Q12W) SC, with an additional 200 mg SC dose at Week 4, or placebo. To maintain blinding, subjects randomized to the daxdilimab 200 mg Q12W SC dosing regimen will receive SC placebo injections on dosing visits outside the Q12W schedule. Randomization are stratified by SLE Disease Activity Index 2000 (SLEDAI-2K) total score at Screening (≥10 or ≤10) and prednisone or equivalent oral glucocorticoid (OGC) dose at Baseline (Day 1) (≥10 mg or ≤10 mg).

The study will comprise a screening period of approximately 4 weeks (Days-28 to −1), Randomization on Day 1, treatment and assessments through Week 48, and a Safety Follow-Up (SFU) period of 8 weeks (through Week 56). Under exceptional circumstances such as delayed laboratory results, drug washout, or the impact of COVID-19, the Screening period may be increased by 2 weeks, upon approval by the Medical Monitor. The study are conducted on an outpatient basis. For all administrations, IP are administered by site staff in the clinic and the subject are observed for at least 60 minutes after the first and second doses. Subjects who prematurely stop dosing prior to Week 44 are followed through Week 56. Subjects will not automatically be removed from the study if any administration of IP is missed.

A long-term extension (LTE) study for safety and efficacy may be offered as part of a separate protocol, to subjects who complete the Week 48 visit. Subjects who enter the LTE study directly after completion of the Week 48 visit will not complete the visits of the SFU period.

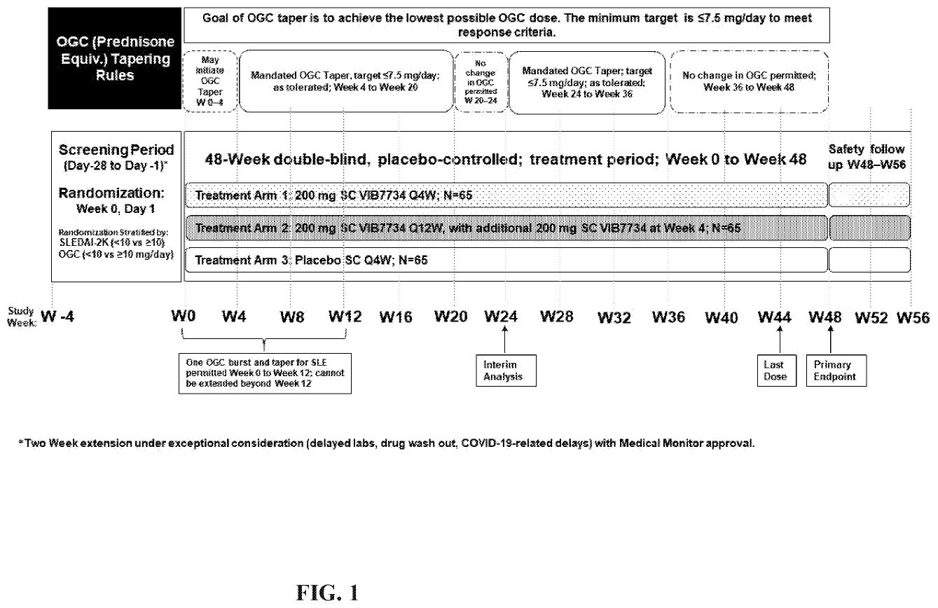

The study design is summarized in FIG. 1.

The primary objective is to evaluate the effect of daxdilimab compared to placebo in reducing SLE disease activity at Week 48 in subjects treated with SoC therapy. The primary objective are measured according to the following criteria.
Proportion of subjects achieving a BILAG-Based Composite Lupus Assessment (BICLA) response and an OGC dose≤7.5 mg/day and ≤Day 1 dose of prednisone or equivalent at Week 48. Subjects will have BICLA and oral glucocorticoid assessed at week 48.
The BICLA response is defined as meeting all the following conditions as compared to Baseline (Day 1):
   BILAG 2004 Index improvement (all Baseline [Day 1] BILAG A improving to B/C/D, all Baseline [Day 1] BILAG B to C/D, and ≤1 new BILAG B and no new BILAG A.
   No deterioration in SLEDAI-2K total score.
   No significant worsening in PGA score (≤10% increase).
   No use of restricted medications beyond the protocol-allowed threshold before assessment.
   No discontinuation of IP.

The efficacy objectives of the study are as follows:
To evaluate the effect of daxdilimab compared with placebo to reduce cutaneous disease activity at Week 12.
   Evaluation will measure proportion of subjects with CLASI-A score ≥10 at baseline (Day 1) who achieve ≥50% reduction from baseline (Day 1) in CLASI-A score at week 12. Scoring will consist of (1) inflammatory activity of the disease; and (2) damage done by the disease.
To evaluate the effect of daxdilimab compared with placebo to reduce SLE disease activity at Week 48.
   Evaluation will measure proportion of subjects achieving an SRI-4 response and an OGC dose ≤7.5 mg/day and ≤ baseline (Day 1) dose of prednisone or equivalent at week 48. SRI-4 (SLE responder index) refers to meeting all criteria compared to baseline, (e.g. no worsening of symptoms).
To evaluate the effect of daxdilimab compared with placebo on sustained OGC reduction from Week 36 to Week 48.
   Evaluation will measure the proportion of subjects at OGC ≥10 mg prednisone or equivalent at baseline (Day 1) who achieve an OCG of ≤7.5 mg/day prednisone or equivalent at week 36 to week 48.
To evaluate the effect of daxdilimab compared with placebo to achieve low disease activity at Week 48.
   Evaluation will measure the proportion of subjects achieving Lupus Low Disease Activity State (LLDAS) at week 48. LLDAS is a composite measure of SLE disease activity that measures 5 criteria: (1) SLEDAI-2K≤4, with no activity in major organ systems, (2) no new lupus disease activity, (3) PGA≤1 (scale 0 to 3), (4) current prednisone or equivalent dose≤7.5 mg daily, (5) tolerated maintenance doses of immunosuppressive drugs, and approved biological agents.

### Exploratory Objectives

(1) To evaluate the effect of VIB7734 compared with placebo on reduction in occurrence of moderate to severe flares through Week 48 as determined by Annualized flare rate. A flare is defined as either ≥1 new BILAG A or ≥2 new BILAG B items compared to the previous visit. (2) To evaluate the effect of VIB7734 compared with placebo on various disease activity measures at Week 48 and over time as determined by BICLA, BILAG 2004 Index, SRI-4, SRI-5, SRI-6, SRI-7, or SRI-8, SLEDAI-2K, CLASI, OGC dose, Joint Count, Major Clinical Response, Partial Clinical Response, Minimal Disease Activity, disease remission, and exposure-response relationship. (3) To explore potential associations of genetic variations, gene expression, and profiles of circulating proteins with VIB7734 response as determined by Change in levels of exploratory biomarkers over time. (4) To evaluate the effect of VIB7734 compared with placebo on participant-reported health-related quality of life and health assessment at Week 48 as determined by Fatigue Scale for Motor and Cognitive Functions, Hospital and Anxiety Depression Scale, Cambridge Neuropsychological Test Automated Battery, Patient Global Assessment, Patient Global Impression of Change, and Lupus Quality of Life. (5) To evaluate the effect of VIB7734 compared with placebo on organ damage at Week 48 as determined by Change in Systemic Lupus International Collaborating Clinics/American College of Rheumatology Damage Index.

### Pharmacokinetic Pharmacodynamic Immunogenicity Objectives

To characterize the pharmacokinetics, pharmacodynamics, and immunogenicity of VIB7734 as determined by VIB7734 concentrations, change in pDCs, and antidrug antibodies rate.

### Inclusion Criteria:

To be included in this study, each subject must satisfy all the following criteria:
1. Age ≥18 years to ≤70 years at the time of signing the informed consent form (ICF).
2. Willing and able to understand and provide written informed consent prior to any study-related procedures and to comply with all study requirements and complete study assessments.
3. Fulfill the 2019 European League Against Rheumatism/American College of Rheumatology Classification Criteria for SLE (Aringer et al, 2019).
4. Disease duration of at least 6 months from the time of diagnosis at the time of signing the ICF
5. Active SLE as indicated by presence of all the following:
   SLEDAI-2K total score ≥6 at Screening, excluding fever, SLE headache, or organic brain syndrome.
   SLEDAI-2K total score ≥4, excluding points attributable to any urine or laboratory results, immunologic measures, fever, SLE headache, or organic brain syndrome at Screening and Baseline (Day 1).
   At least 1 of the following BILAG 2004 Index levels of disease at Screening:
      BILAG A disease in ≥1 organ system.
      BILAG B disease in ≥2 organ systems.
   PGA score ≥1 on a 0 to 3 visual analog scale (VAS) at Screening.
6. Have at least 1 of the following at Screening per central lab:
   antinuclear antibody (ANA)≥1:80.
   Anti-dsDNA antibodies elevated to above normal range as established by the central laboratory (i.e., positive results).
   Anti-Smith antibodies elevated to above normal (i.e., positive results).
7. Ongoing treatment for SLE defined as (a) or (b):
   a. Treatment with a disease modifying anti-rheumatic drug (DMARD) or immunosuppressive medication: Any of the following medications each administered at conventional anti-rheumatic doses for treatment of SLE for at least 12 weeks before Screening (unless discontinued or dose adjusted for documented drug-related toxicity or size/weight), and at a stable dose (including route of administration) for a minimum of 8 weeks prior to Screening and maintained through Baseline (Day 1):
      i. Antimalarial
         Chloroquine
         Hydroxychloroquine
         Quinacrine
      ii. Azathioprine (AZA) or 6-mercaptopurine (6-MP)
      iii. Leflunomide
      iv. Mycophenolate mofetil (MMF) or mycophenolic acid (MPA)
      v. Methotrexate (MTX) (subjects must be on concomitant folic or folinic acid supplementation if using MTX)
      vi. Voclosporin (if approved for treatment)
      vii. GCs are permitted but not required if a subject is receiving at least 1 other medication listed above. If GCs are used in combination with allowed DMARDs or immunosuppressants, they must be at an average daily dose of PO prednisone≤40 mg (or prednisone equivalent) for a minimum of 2 weeks prior to Screening and at a stable dose for a minimum of 2 weeks prior to Screening. In addition, the dose of OGC must be kept stable for a minimum of 2 weeks prior to Randomization. Daily dosing or alternate day dosing of PO prednisone (or prednisone equivalent) is allowed.
   b. Treatment with OGC monotherapy (without the concomitant use of DMARDs or immunosuppressants):
      i. Average daily dose of PO prednisone ≥10 mg but ≤40 mg (or prednisone equivalent) for a minimum of 4 weeks prior to Screening and at a stable dose for a minimum of 2 weeks prior to Screening. In addition, the dose of OGC must be kept stable for a minimum of 2 weeks prior to Randomization. Daily dosing or alternate day dosing of PO prednisone (or prednisone equivalent) is allowed.
8. Women of childbearing potential must have a negative serum pregnancy test at screening and a negative urine pregnancy test at Randomization. Women of childbearing potential are defined as those who are not surgically sterile (i.e., surgical sterilization includes bilateral tubal ligation, bilateral oophorectomy, or hysterectomy) or those who are not postmenopausal (defined as 12 months with no menses without an alternative medical cause and a follicle-stimulating hormone [FSH] within the postmenopausal range as established by the central laboratory, unless on postmenopausal hormone replacement therapy).
   Women of childbearing potential who are sexually active with a non-sterilized male partner must agree to use a highly effective method of contraception from signing of the informed consent, and must agree to continue using such precautions through the end of the study follow-up or 3 months (approximately 5 half-lives) following the last dose of study drug in the case of early withdrawal from the study. A decision about contraception after this point should be made by the subject and her regular healthcare providers.
   Sustained abstinence is an acceptable practice; however periodic abstinence, the rhythm method, and the withdrawal method are not acceptable methods of contraception.
   Note that because mycophenolate affects the metabolism of hormonal contraceptives and may reduce their effectiveness in women receiving MMF or MPA who are using hormonal contraceptives for birth control, the subject must employ an additional contraceptive method (e.g., barrier method).
9. Non-sterilized male subjects who are sexually active with a woman partner of childbearing potential must agree to use a condom with spermicide from Randomization and until 3 months (approximately 5 half-lives) after receipt of the last dose. Because a male condom with spermicide is not a highly effective contraception method, it is strongly recommended that male subjects advise their women partners of childbearing potential to use a highly effective method of contraception throughout this period.

### Exclusion Criteria:

1. Individuals involved in the conduct of the study, their employees, or immediate family members of such individuals.
2. Any condition that, in the opinion of the Investigator, would interfere with evaluation of the IP or interpretation of subject safety or study results.
3. History of allergy, hypersensitivity reaction, or anaphylaxis to any component of the IP or to a previous mAb or human Ig therapy.
4. Participation in another clinical study with an IP within 4 weeks prior to Day 1 or within 5 published half-lives, whichever is longer.
5. Lactating or pregnant women or women who intend to become pregnant anytime from signing the ICF through 6 months after receiving the last dose of IP.
6. History of drug or alcohol abuse that, in the opinion of the Investigator, might affect subject safety or compliance with visits, or interfere with other study assessments.
7. Major surgery within 8 weeks prior to Screening or elective surgery planned from Screening through Day 393.
8. Spontaneous or induced abortion, still or live birth, or pregnancy ≤4 weeks prior to Screening.
9. Known history of a primary immunodeficiency or an underlying condition such as known human immunodeficiency virus (HIV) infection, a positive result for HIV infection per central laboratory, splenectomy, or any underlying condition that in the opinion of the Investigator significantly predisposes the subject to infection.
10. At Screening, any of the following per central laboratory (tests may be repeated once within the same Screening period to confirm results prior to Randomization):
   AST≥2.5×ULN
   ALT≥2.5×ULN
   Total bilirubin≥1.5×ULN (unless due to Gilbert's syndrome)
   Serum IgG≤600 mg/dL (or ≤6 g/L)
   Neutrophil count≤1000/μL (or ≤1.0×10⁹/L) or ≤500/μL (≤0.5×10⁹/L) if due to active SLE
   Platelet count≤50,000/μL (or ≤50×10⁹/L) or ≤250,000/μL (≤25×10⁹/L) if due to active SLE
   Hemoglobin≤8 g/dL (or ≤80 g/L) or ≤7 g/dL (≤70 g/L) if due to active SLE
   Glycosylated hemoglobin≥8% (or ≥0.08)
   Total lymphocyte count≤200 cells/mm³
   Glomerular filtration rate≤30 mL/min/1.73 m²
   Spot UPCr≥3 mg/mg (greater than 339 mg/mmol)
11. Confirmed positive test for hepatitis B serology defined as:
   Hepatitis B surface antigen (HBsAg), or
   Hepatitis B core antibody (HBcAb) AND hepatitis B virus (HBV) DNA detected above the lower limit of quantitation (LLOQ) by reflex testing by the central laboratory at Screening.
   Note that subjects who are HBcAb positive at Screening are tested every 3 months for HBV DNA. Study drug are discontinued if the subject's HBV DNA levels are confirmed to exceed the LLOQ as per the central laboratory.
12. Positive test for hepatitis C virus antibody.
13. Active TB, or a positive IFN-gamma release assay (IGRA) test at Screening, unless documented history of appropriate treatment for active or latent TB. Subjects with an indeterminate IGRA test result can repeat the test, but if the repeat test is also indeterminate, they are excluded.
14. Any severe herpes virus family infection (including Epstein-Barr virus, cytomegalovirus [CMV]) at any time prior to Randomization, including, but not limited to, disseminated herpes, herpes encephalitis, recent recurrent herpes zoster (defined as 2 episodes within the last 2 years), or ophthalmic herpes.
15. Any herpes zoster, CMV, or Epstein-Barr virus infection that was not completely resolved 12 weeks prior to Randomization.
16. Any of the following within 30 days prior to signing the ICF and though Randomization:
   Clinically significant active infection in the opinion of the Investigator, including ongoing, and chronic infection requiring antibiotics or antiviral medication (chronic nail infections are allowed).
   Any infection requiring hospitalization or treatment with IV anti-infectives.
   A subject with a documented positive SARS COV-2 test may be rescreened at least 2 weeks after a positive test if the subject is asymptomatic and at least 3 weeks after symptomatic COVID-19 illness.
17. Opportunistic infection requiring hospitalization or parenteral antimicrobial treatment within 2 years prior to Randomization.
18. Any acute illness or evidence of clinically significant active infection.
19. History of clinically significant cardiac disease including unstable angina and/or myocardial infarction and/or congestive heart failure within 6 months prior to randomization. Or any cardiac condition including but not limited to: inadequately controlled arrhythmia, presence of clinically significant abnormality on ECG if, in the opinion of the Investigator, it would increase the risk of study participation.
20. History of cancer within the past 5 years, except as follows:
   In situ carcinoma of the cervix treated with apparent success with curative therapy ≥12 months prior to Screening, or
   Cutaneous basal cell or squamous cell carcinoma treated with apparent success with curative therapy.
21. Receipt of a live-attenuated vaccine within 4 weeks prior to Day 1. Administration of inactivated (killed) vaccines is acceptable.
22. Subject should be assessed for epidemiologic risk of COVID-19 (i.e., recent exposure, high-risk housing) and for health-related risk of COVID-19 severity based on current understanding of risk factors for severe disease when making a decision regarding the individual's risk of participation. Subjects who have COVID-19 or other significant infection, or in the judgment of the Investigator, may be at a high risk of COVID-19 or its complications should not be randomized.
23. Any one of: active LN or active severe or unstable neuropsychiatric SLE (e.g., aseptic meningitis, cerebral vasculitis, myelopathy, demyelination syndromes [ascending, transverse, acute inflammatory demyelinating polyradiculopathy], acute confusional state, impaired level of consciousness, psychosis, acute stroke or stroke syndrome, cranial neuropathy, status epilepticus, cerebellar ataxia, and mononeuritis multiplex) where, in the opinion of the Investigator or Medical Monitor, protocol-specified SoC is insufficient and utilization of a more aggressive therapeutic approach, such as IV cyclophosphamide, high-dose IV pulse GC therapy, MMF of ≥3 gm/day (MPA of ≥2.16 gm/day), or an increase from baseline dose of MMF/MPA and/or other treatments not permitted in the protocol, may be indicated.)
24. Diagnosis of non-SLE vasculitis syndrome, mixed connective tissue disease, or rheumatic (overlap) syndrome.
25. Use of immunosuppressants, biologics, and DMARDS within the protocol defined washout periods.

### Example 2: An Open-Label Extension Study to Evaluate the Long-Term Safety and Tolerability of Daxdilimab in Subjects with Systemic Lupus Erythematosus (SLE)

A Phase 2, multicenter, open-label extension (OLE) study is conducted to evaluate the long-term safety and tolerability of daxdilimab in subjects completing the treatment period of the RECAST SLE clinical study. The RECAST SLE study is a Phase 2 randomized, double-blind, placebo-controlled efficacy and safety study of daxdilimab for the treatment of moderately-to-severely active systemic SLE. The study enrolls approximately up 195 subjects. The SLE open-label extension (OLE) study (HZN-DAX-204) is a long-term OLE study of daxdilimab plus standard of care in subjects who complete the treatment period of the RECAST SLE protocol. Irrespective of their assigned treatment in the RECAST SLE study, all subjects participating in the OLE are treated with daxdilimab subcutaneously (SC) every 12 weeks (Q12W) in addition to their standard-of-care SLE therapy.

To allow for the continuous dosing of the subjects, the start of this OLE study should occur immediately after the completion of the RECAST SLE treatment period (ie, first OLE dosing [Day 1] coinciding with Week 48/Visit 14 of the RECAST SLE study).

The primary and secondary objectives and associated endpoints are detailed below. In brief, the primary objective of this study is to evaluate the long-term safety and tolerability of 200 mg daxdilimab Q12W in adult subjects with moderately-to-severely active SLE. This are assessed by summarizing adverse events (AEs), serious adverse events (SAEs), and AEs of special interest (AESIs). Local injection site tolerability, vital signs, physical examinations, electrocardiograms, and clinical laboratory tests will also be performed to support safety findings.

TABLE 3
Primary, Secondary, and Exploratory Objectives

| | |
|---|---|
| Objectives | Endpoints |

Primary Objective

| | |
|---|---|
| To evaluate the long-term safety and | Incidence of AEs, SAEs, and AESIs |
| tolerability of 200 mg Q12W daxdilimab | |

Secondary Objectives

| | |
|---|---|
| To characterize the PK, PD, and | Daxdilimab concentrations, change in pDCs, and ADA rate |
| immunogenicity of daxdilimab | |

Exploratory Objectives

| | |
|---|---|
| To evaluate the impact of 200 mg Q12W | Proportion of subjects achieving an improvement in |
| daxdilimab on maintaining responses | SLEDAI-2K, as measured from baseline SLEDAI-2K in the |
| | RECAST SLE study and not higher than their Day 1 |
| | SLEDAI-2K in HZNP-DAX-204 |
| | Proportion of subjects not having a flare based on the mSFI and |
| | SLEDAI-2K ≤ 6 |
| | Number of flares throughout the study as measured by the |
| | mSFI |
| | Proportion of subjects that achieve ≤ 2.5 mg/day of OGC usage |
| | Proportion of subjects receiving rescue medication |
| | Proportion of subjects achieving LLDAS over time |
| | Proportion of time spent in LLDAS throughout the study |
| | Change from baseline in SDI |
| | Change in exploratory biomarkers over time |

ADA = anti-drug antibodies; AE = adverse event; AESI = AE of special interest; LLDAS = Lupus Low Disease Activity State; mSFI = modified SLEDAI-2K Flare Index; OGC = oral glucocorticoid; PD = pharmacodynamic(s); pDC = plasmacytoid dendritic cell; PGA = Physician Global Assessment; PK, pharmacokinetic(s); Q12W = every 12 weeks; RECAST SLE = Phase 2 Randomized, Double-Blind, Placebo-Controlled Efficacy and Safety Study of daxdilimab for the Treatment of Moderate to Severely Active Systemic Lupus Erythematosus; SDI = Systemic Lupus International Collaborating Clinics (SLICC)/American College of Rheumatology (ACR) Damage Index; SAE = serious AE; SLE = systemic lupus erythematosus; SLEDAI-2K = Systemic Lupus Erythematosus Disease Activity Index 2000

### Treatment

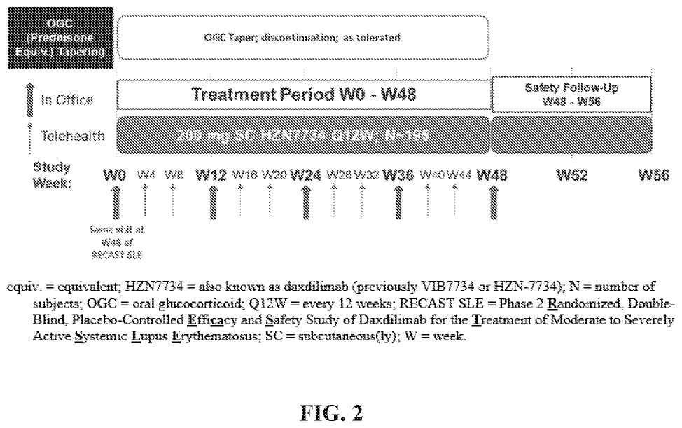

Subjects are treated with open-label daxdilimab 200 mg at the study site every 12 weeks (Q12W) subcutaneously (SC) for 48 weeks, see FIG. 2. Daxdilimab is to be administered as two 1.0-mL SC injections. A summary of the treatment is provided in Table 4.

TABLE 4
Description of Investigational Product and Dosing

| Description | Investigational Product |
|---|---|
| Product name | Daxdilimab |
| Dosage form | 1 mL of 100 mg/mL daxdilimab containing 20 mM L-histidine/L-histidine HCl, |
| | 240 mM sucrose, 0.02% (w/v) polysorbate 80, pH 6.0 |
| Source of procurement | Horizon Therapeutics |
| Unit dose | 200 mg dose, administered as 2 × 1.0 mL SC injections |
| strength(s)/Dosage level(s) | |
| Route of Administration | SC, Q12W for 48 weeks (Weeks 0 [Day 1], 12, 24, 36, and 48) |
| and frequency | |
| Physical description | The drug product are provided as a sterile liquid in a 2R glass vial with a nominal |
| | fill volume of 1.0 mL of 100 mg/mL. |
| Dosing instructions | Daxdilimab are administered by clinic staff trained in best practices for SC |
| | administration of treatments. More details on the administration method are |
| | described in the study manual. |

Q12W = once every 12 weeks; SC = subcutaneous; w/v = weight/volume

During the treatment period, a telehealth visit (e.g., phone or video call) will also be performed every 4 weeks between in-clinic visits to assess the subject's current disease status, the OGC tapering schedule, safety, and concomitant medications. After the treatment period (Week 0 to Week 48), the study subjects will enter an 8-week safety follow-up period (Week 48 to Week 56), see schedule of activities at Table 5.

TABLE 5
Exemplary Schedule of Activities

| | Study Day | | | | | | | | | | |
|---|---|---|---|---|---|---|---|---|---|---|---|
| | Treatment | | | | | | | | | Follow-Up | |
| | 1 | 29/57 | 85 | 113/141 | 169 | 197/225 | 253 | 281/309 | 337 | 365 | 393 |
| | Week (window) | | | | | | | | | | |
| | | 4/8 | 12 | 16/20 | 24 | 28/32 | 36 | 40/44 | 48/ET | 52 | 56 |
| | 0 | (±3 d) | (±7 d) | (±3 d) | (±7 d) | (±3 d) | (±7 d) | (±3 d) | (±7 d) | (±7 d) | (±7 d) |
| | Procedure/Visit Number | | | | | | | | | | |
| | V1 ^{a} | V2/V3 ^{b} | V4 | V5/V6 ^{b} | V7 | V8/V9 ^{b} | V10 | V11/V12 ^{b} | V13 | V14 | V15 |
| General Assessments/ | | | | | | | | | | | |
| Procedures | | | | | | | | | | | |
| ICF | X | | | | | | | | | | |
| Eligibility review | X | | | | | | | | | | |
| Review medical history | X | | | | | | | | | | |
| and add any | | | | | | | | | | | |
| AEs/SAEs/AESIs from | | | | | | | | | | | |
| RECAST SLE (if | | | | | | | | | | | |
| appropriate) | | | | | | | | | | | |
| 12-lead ECG (after | X | | | | X | | | | X | | X |
| 10 minutes rest in | | | | | | | | | | | |
| supine position) ^{c} | | | | | | | | | | | |
| Vital signs and weight ^{d} | X | | X | | X | | X | | X | X | X |
| Physical examination ^{e} | X | | X | | X | | X | | X | X | X |
| SDI | | | | | X | | | | X | | |
| SLEDAI-2K, mSFI, | X | | X | | X | | X | | X | X | X |
| CLASI, PGA, and Joint | | | | | | | | | | | |
| Count ^{f} | | | | | | | | | | | |
| Laboratory Assessments | | | | | | | | | | | |
| Urine pregnancy test (in | X | | X | | X | | X | | X | X | X |
| women of childbearing | | | | | | | | | | | |
| potential) | | | | | | | | | | | |
| FSH ^{g} | X | | | | | | | | | | |
| Hepatitis B DNA | X | | X | | X | | X | | X | | X |
| testing ^{h} | | | | | | | | | | | |
| Routine hematology and | X | | X | | X | | X | | X | X | X |
| chemistry | | | | | | | | | | | |
| HbA1c | X | | | | X | | | | X | | X |
| Lipids (after an 8-hour | X | | | | X | | | | X | | |
| fast; water allowed) ⁱ | | | | | | | | | | | |
| Urinalysis and spot | X | | X | | X | | X | | X | X | X |
| UPCr ^{j} | | | | | | | | | | | |
| Confirmatory tests for | X | | X | | X | | X | | X | X | X |
| hemolytic anemia ^{k} | | | | | | | | | | | |
| C3 and C4 | X | | X | | X | | X | | X | X | X |
| Anti-dsDNA antibodies | X | | X | | X | | X | | X | X | X |
| Anti-Smith antibodies | X | | | | | | | | X | | |
| ANA, anti-RNP, anti- | X^{l} | | | | | | | | X | | |
| SSA, anti-SSB, | | | | | | | | | | | |
| rheumatoid factor | | | | | | | | | | | |
| PT/INR and PTT | X | | X | | X | | X | | X | | X |
| Serum IgG, IgA, IgM | X | | X | | X | | X | | X | | X |
| Serum IFNα | X | | X | | X | | X | | X | | X |
| hs-CRP | X | | | | X | | X | | X | X | X |
| pDC FACS | X | | X | | X | | X | | X | | |
| Serum exploratory | X | | X | | X | | X | | X | | X |
| biomarkers | | | | | | | | | | | |
| Plasma exploratory | X | | X | | X | | X | | X | | X |
| biomarkers | | | | | | | | | | | |
| Whole blood | X | | X | | X | | X | | X | | X |
| transcriptomics | | | | | | | | | | | |
| PBMC cytometry | X | | | | X | | | | X | | X |
| Blood MxA | X | | | | X | | | | X | | X |
| Daxdilimab PK (serum) | X | | X | | X | | X | | | | X |
| Daxdilimab ADA | X | | X | | X | | X | | X | | X |
| Questionnaires ^{m} | | | | | | | | | | | |
| PtGA | X | | X | | X | | X | | X | | X |
| PGIC | X | | X | | X | | X | | X | | X |
| LupusQoL | X | | X | | X | | X | | X | | X |
| Investigational Product | | | | | | | | | | | |
| Administration | | | | | | | | | | | |
| IP administration | X | | X | | X | | X | | X | | |
| Safety | | | | | | | | | | | |
| Steroid Taper | X | X | X | X | X | X | X | X | | | |
| Discussion | | | | | | | | | | | |
| Local injection | X | | X | | X | | X | | X | | |
| tolerability | | | | | | | | | | | |
| Concomitant | X | X | X | X | X | X | X | X | X | X | X |
| medications | | | | | | | | | | | |
| AEs, SAEs, and/or | X | X | X | X | X | X | X | X | X | X | X |
| AESIs | | | | | | | | | | | |

ADA = anti-drug antibody(ies); AEs = adverse events; AESI = adverse events of special interest; ANA = antinuclear antibodies; C = complement; CLASI = Cutaneous Lupus Erythematosus Disease Area and Severity Index; d = days; dsDNA = double-stranded DNA; ECG = electrocardiogram; ET = early termination; FACS = fluorescence-activated cell sorting; FSH = follicle-stimulating hormone; HbA1c = glycated hemoglobin A1c; HBV = hepatitis B virus; hs-CRP = high-sensitivity C reactive protein; ICF = informed consent form; IFNα = interferon alfa; Ig = immunoglobulin; INR = international normalized ratio; IP = investigational product; LLOQ = lower limit of quantitation; LupusQoL = Lupus Quality of Life; mSFI = modified SLEDAI-2K Flare Index; MxA = myxovirus resistance protein; PBMC = peripheral blood mononuclear cells; pDC = plasmacytoid dendritic cell; PGA = Physician Global Assessment; PGIC = Patient Global Impression of Change; PK = pharmacokinetics; PT = prothrombin time; PtGA = Patient Global Assessment; PTT = partial thromboplastin time; RECAST SLE = Phase 2 Randomized, Double-Blind, Placebo-Controlled Efficacy and Safety Study of Daxdilimab for the Treatment of Moderate to Severely Active Systemic Lupus Erythematosus; RNP = ribonucleoprotein; SAEs = serious adverse events; SDI = Systemic Lupus International Collaborating Clinics (SLICC)/American College of Rheumatology (ACR) Damage Index; SLE = systemic lupus erythematosus; SLEDAI-2K = Systemic Lupus Erythematosus Disease Activity Index 2000; SSA = Sjögren's syndrome-related antibody A; SSB = Sjögren's syndrome-related antibody B; UPCr = urine protein:creatinine ratio; V = visit
^{a} Any procedure that is also completed on RECAST SLE V14 (if performed on the same day as VI for this study should be utilized for this study and not repeated.
^{b} Visits 2, 3, 5, 6, 8, 9, 11, and 12 are telehealth visits (eg, phone or video call).
^{c} ECGs should be performed after vital signs are collected.
^{d} Vital signs include systolic and diastolic blood pressure obtained after at least 5 minutes at rest in a seated position, heart rate, respiratory rate (breaths/min), and body temperature.
^{e} A full physical examination should be performed at Visit 1 and Visit 13. A focused physical examination are performed at all other in-clinic visits, and these should always include assessment of head, ears, eyes, nose, throat, lungs, heart, abdomen, skin, and extremities.
^{f} Additional assessments, (eg, ECG or chest X-ray) should be performed as needed to fully obtain information needed for the SLEDAI-2K assessments.
^{g} This test is conditional and can be performed at any in-clinic visit throughout the study if a female subject becomes postmenopausal during the OLE study (ie, 12 months with no menses without an alternative medical cause, unless on postmenopausal hormone replacement therapy).
^{h} Reflex DNA testing if isolated hepatitis B core positive in RECAST SLE. Investigational product are discontinued if the subject's HBV DNA levels are confirmed to exceed the LLOQ as per the central laboratory.
ⁱ This visit is recommended to occur in the morning.
^{j} Aim to collect urine at the same time of day, if possible. Urine collection can be postponed for up to 14 days in women with menstrual bleeding or a urinary tract infection at the scheduled visit.
^{k} Serum aliquots are used if needed to confirm suspected hemolytic anemia.
^{l} ANA only
^{m} Questionnaires are strongly recommended to be completed before any other procedures are performed.

### Inclusion Criteria

(1) Willing and able to understand and provide written informed consent prior to any study-related procedures and to comply with all study requirements and complete study assessments. (2) Must have qualified for and received IP (daxdilimab or placebo) and complete the treatment period (through Day 337) in the RECAST SLE study. Subjects who discontinued early from IP in RECAST SLE are not eligible for this study. (3) Women of childbearing potential must have a negative urine pregnancy test on Day 1. Women of childbearing potential are defined as those who are not surgically sterile (ie, surgical sterilization includes bilateral salpingectomy, bilateral oophorectomy, or hysterectomy) or those who are not postmenopausal (defined as 12 months with no menses without an alternative medical cause and a follicle-stimulating hormone [FSH] within the postmenopausal range as established by the central laboratory during the screening period of the RECAST SLE study, unless on postmenopausal hormone replacement therapy). If a female subject becomes postmenopausal during the study (ie, 12 months with no menses without an alternative medical cause, unless on postmenopausal hormone replacement therapy), a FSH test are performed at the central laboratory. If the FSH level is within the postmenopausal range, the female subject will not be required to use contraception after that. Women of childbearing potential who are sexually active with a nonsterilized male partner must agree to use a highly effective method of contraception from signing of the ICF and must agree to continue using such precautions through the end of the study follow-up or 3 months (approximately 5 half-lives) following the last dose of IP in the case of early withdrawal from the study, and refrain from egg retrieval/egg donation during this period. After this point, a decision about contraception should be made by the subject and her regular healthcare providers. Female subjects who participate in the SLE OLE are expected to maintain the same form of contraception they used during the RECAST SLE study. Note that because mycophenolate affects the metabolism of hormonal contraceptives and may reduce their effectiveness in women receiving MMF or mycophenolic acid (MPA) who are using hormonal contraceptives for birth control, the subject must employ an additional contraceptive method (eg, barrier method). (4) Nonsterilized male subjects who are sexually active with a woman partner of childbearing potential must agree to use a condom with spermicide from Day 1 and until 3 months (approximately 5 half-lives) after receipt of the last dose. Because a male condom with spermicide is not a highly effective contraception method, it is strongly recommended that male subjects advise their women partners of childbearing potential to use a highly effective method of contraception throughout this period.

### Exclusion Criteria

(1) Any condition or change during the RECAST SLE study that in the opinion of the Investigator or the Sponsor would interfere with evaluation and interpretation of subject safety or alter the risk-benefit associated with IP administration. (2) Participation in another clinical study with an IP during the RECAST SLE study period. (3) Planned elective surgeries that in the opinion of the Investigator or the Sponsor would interfere with evaluation and interpretation of subject safety. (4) Any herpes zoster, cytomegalovirus, or Epstein-Barr virus infection that was not completely resolved prior to Visit 1. (5) Clinically significant active infection at Visit 1, in the opinion of the Investigator, including ongoing and chronic infection requiring antibiotics or antiviral medication (chronic nail infections are allowed). (6) Pregnant or lactating females. Prior and Concomitant Therapy Criteria: (7) Receipt of any prohibited medication during the RECAST SLE study period. Receipt of any restricted medications during the RECAST SLE study period must be discussed with the Sponsor's Medical Monitor and agreed upon prior to enrollment into this study.

### Reasons to Discontinue

An individual subject will not receive any further Daxdilimab if any of the following occur in the subject: (1) Receipt of any medications or therapies not allowed; (2) A Grade 3 or higher allergic reaction to the Daxdilimab; (3) A Grade 3 or higher infection considered related to the Daxdilimab; (4) Other adverse events that contraindicates further dosing in the opinion of the Investigator and/or the Sponsor, Medical Monitor; (5) Withdrawal of consent from further treatment with Daxdilimab; (6) Subject is determined to have met one or more of the exclusion criteria or failed to meet all the inclusion criteria for study participation and there is a potential safety risk associated with continuation identified upon consultation with the Medical Monitor; (7) Pregnancy or a decision to become pregnant; (8) Any of the following liver function abnormalities: Alanine aminotransferase (ALT) or aspartate aminotransferase (AST)≥8× upper limit of normal (ULN), ALT or AST≥5×ULN for more than 2 weeks, ALT or AST≥3×ULN and total bilirubin≥2×ULN or international normalized ratio≥1.5 without alternative explanation, ALT or AST≥3×ULN with the appearance of fatigue, nausea, vomiting, right upper quadrant pain or tenderness, fever, rash, and/or eosinophilia (≥5%).

### Concomitant Therapy-Concomitant Medications for Systemic Lupus Erythematosus Standard of Care During the Study

Immunosuppressants, if given during the RECAST SLE study, must remain stable during the treatment period except for oral glucocorticoid (OGC) and nonsteroidal anti-inflammatory drugs (NSAIDs). Background therapy may only be changed for documented safety issues. The toxicity/event must be confirmed as a documented AE. The dose can be returned to the OLE baseline (Day 1) level if the toxicity/event resolves and if clinically indicated. Initiation of any new immunosuppressant or immunomodulator therapy or increase in dose above the level used on Day 1 of the OLE may result in withdrawal of the subject from the IP (per Sponsor's Medical Monitor discretion).

Glucocorticoids (GCs). Oral GCs are permitted but not required for participation in the study. Oral GCs other than prednisone may be used orally (PO) at equivalent doses. It is strongly encouraged that Investigators taper the OGC dose as much as tolerated after a subject enrolls into the study, as a goal of this OLE is to determine the lowest clinically appropriate level of steroids, including steroid discontinuation, by Week 36 that can be provided while maintaining responses. Guidance for taper and bursts is outlined below. During the study, Investigators may reach out to discuss any taper or burst scenario with the Medical Monitor as needed.

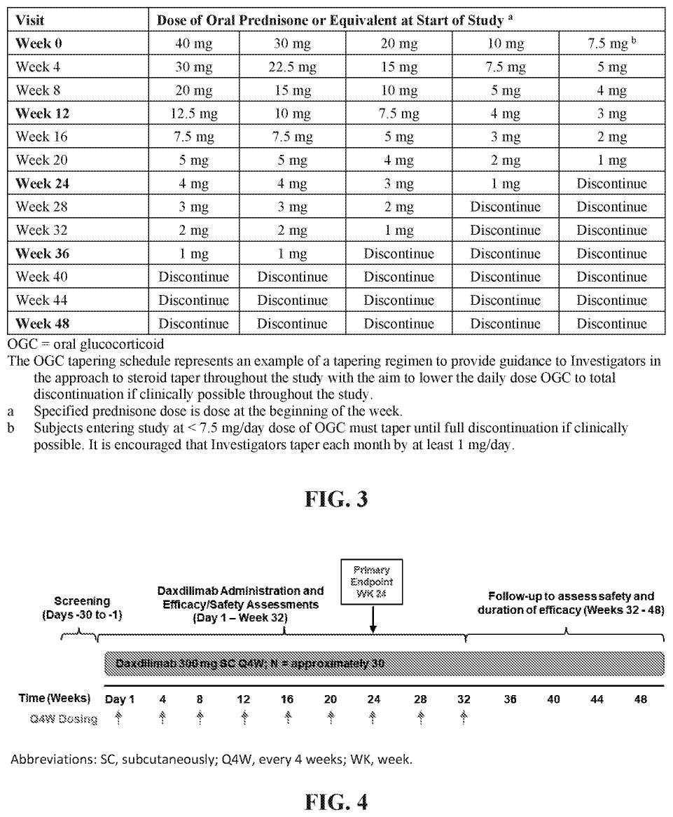

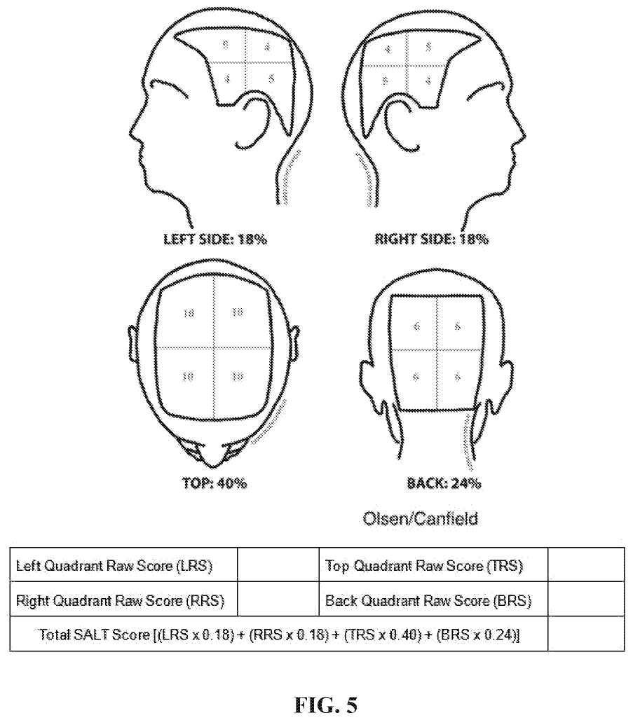

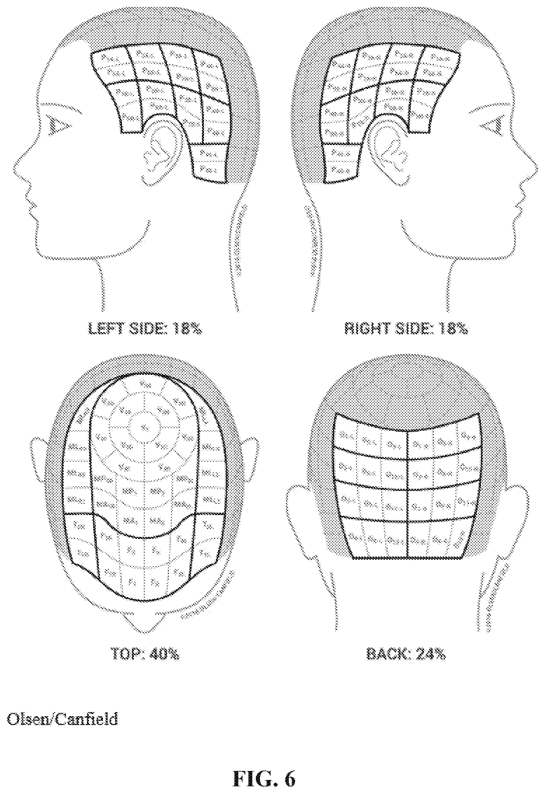

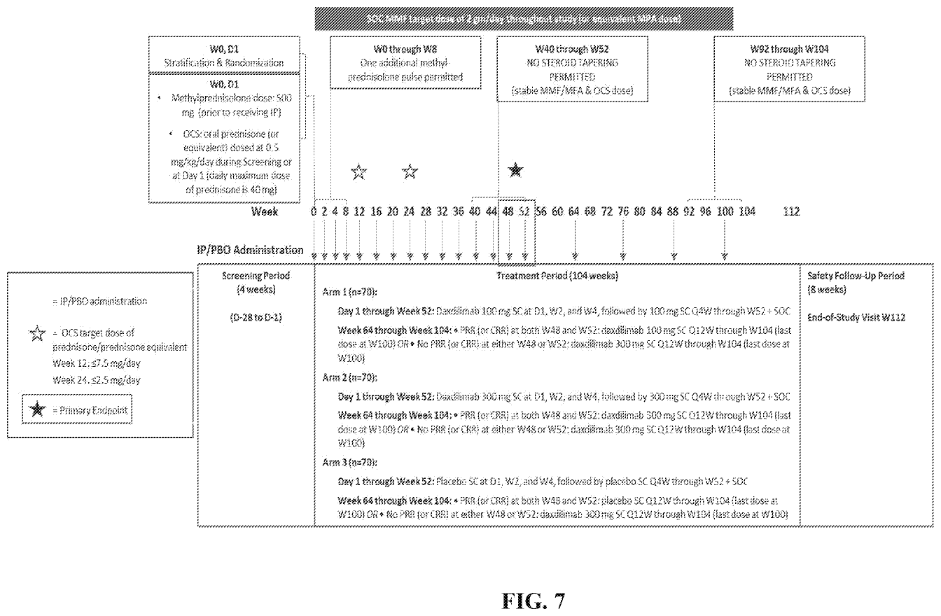

Oral Glucocorticoid Tapering Guidance. Oral GC tapering must be started by Week 4 of the clinical study. An example of a suggested OGC tapering regimen is provided in FIG. 3. However, due to variability in subject responses to OGC treatment and tolerability of taper, Investigators will have flexibility in how the OGC dose is reduced at each visit, however, it is encouraged that Investigators taper each month by at least 1 mg/day. OGC tapering must be attempted unless at least one of the following criteria are met: (1) There are new or worsening organ system(s) affected by SLE. (2) There is an increase in skin involvement. (3) There is new or worsening joint involvement. If there are scenarios outside these listed criteria in which the Investigator deems tapering not appropriate, then the Medical Monitor must be notified and the reason for not tapering are discussed.

Glucocorticoid Burst Guidance. We encourage Investigators to limit the use of GC bursts throughout the clinical study as clinically feasible. GC bursts are allowed at the Investigators discretion.

Increase in Oral Glucocorticoids for Surgery and Prevention of Adrenal Insufficiency. Increases in OGCs for surgery and prevention of adrenal insufficiency are allowed as clinically indicated.

### Other Permitted Medications

Angiotensin-converting enzyme inhibitors and angiotensin receptor blockers: If used during the RECAST SLE study, it is recommended that they be maintained at a stable dose during the OLE study, unless dose change, discontinuation, or initiation is needed for documented safety reasons.

### Anti-COVID Therapeutic Antibodies

Aspirin. Low-dose aspirin (≤350 mg/day) may be used for cardiovascular prophylaxis; this is permitted in addition to use of NSAID as specified below.

Osteoporosis prophylaxis and treatment. Vitamin D and calcium supplementation, and if necessary, treatment of osteoporosis is allowed according to local standard-of-care guidelines.

Herbal supplements may be continued during the study. It is recommended that the dosages and preparations remain stable unless discontinued all together. It is recommended that no herbal supplements be initiated or reinitiated once discontinued during the study.

Medications for the treatment of injection site reactions. These may include topical or systemic antihistamines, topical GCs, paracetamol, or NSAIDs.

NSAIDs. NSAIDs can be used as needed (PRN) during the OLE. Subjects taking a NSAID (including cyclooxygenase 2 inhibitors; topical, prescription, or OTC) on a regular schedule for SLE symptoms at the OLE baseline (Day 1) can continue to do so throughout the study at a stable dose. NSAIDs should not be taken on visit days until all assessments are complete. Subjects may have the dose adjusted during the study for documented toxicity/safety reasons. Any NSAIDs (whether prescription, OTC, or topical) should not be used above the maximum allowable doses per local guidelines.

Opioids. Up to 40 mg/day morphine-equivalent are permitted at a stable dose if present at the OLE baseline (Day 1). Initiation of opioids and/or PRN dosing of opioids after Day 1 of the OLE for SLE is discouraged and should not exceed 40 mg/day or equivalent if prescribed. These may be titrated off as tolerated during the study. Analgesics, including opiates, may be used at stable doses or PRN for temporary relief of symptoms not due to SLE, but then are strongly recommended to be avoided 24 hours prior to each study visit.

Acetaminophen. Short acting acetaminophen (paracetamol) may be initiated or continued for pain control during the study at approved doses. Pain medications should not be used within a minimum of 6 to 12 hours (based on known duration of effect) of a scheduled visit.

Topical therapy for CLE. Concurrent use of topical therapy for CLE (e.g., GCs, pimecrolimus) is permitted. Topical therapy must be the same being used in the 2 weeks prior to the OLE baseline (Day 1), and the dose and frequency of application must be stable. During the study, topical therapy may be reduced or discontinued based on clinical manifestations and Investigator discretion. Should cutaneous skin manifestations reoccur, the same topical therapy may be resumed up to the dose used at the OLE baseline (Day 1). New dermatologic preparations may be used for the duration of the study if needed. It is also recommended that subjects use sunscreen (list as concomitant medication for SLE) and avoid sun exposure during the study. Topical moisturizers are also permitted.

Any medications (other than those prohibited by the protocol) that are considered necessary for the subjects' welfare and will not interfere with the study medication may be given at the Investigator's discretion.

### Prohibited Medications

Biologic immunomodulators (including but not limited to belimumab, abatacept, or rituximab)
Bone marrow, stem cell (eg, mesenchymal stem cells), or solid organ transplant
Cyclophosphamide
GCs
   Intralesional
   Intradermal for alopecia
   Adrenocorticotropic hormone (ACTH) analogs (e.g., Acthar®, Synacthen®)
   dehydroepiandrosterone
   Topical use (may be allowed per the permitted medications) if they are used in accordance with inclusion and exclusion criteria.
Ig therapy
Investigational agents
IPP-201101 (Lupuzor™)
IV corticosteroids ≥1 g methylprednisolone or equivalent
Janus kinase inhibitors (e.g., tofacitinib [Xeljanz®], baricitinib [Olumiant®], upadacitinib [Rinvoq™], filgotinib, peficitinib)
Live or attenuated vaccines, including Bacille-Calmette-Guerin (the Sponsor recommends that Investigators ensure all subjects are up to date with required vaccinations prior to entry into the study)
Minocycline
Plasmapheresis, plasma exchange, or Therakos® photopheresis
Thalidomide and thalidomide derivatives (e.g., lenalidomide [Thalomid®, Revlimid®])
Topical calcineurin or mammalian target of rapamycin (mTOR) inhibitors (eg, pimecrolimus [e.g., Elidel®], sirolimus)
Sulfasalazine
Systemic mTOR inhibitors (e.g., sirolimus [Rapamune®])

### Restricted Medications

As daxdilimab is an investigative immunomodulatory agent, non-protocol-permitted changes to immune modifiers or immunosuppressants on study are strongly discouraged.

If a subject starts any of the following during the treatment period or increases the dose above the OLE baseline (Day 1) dose, the Investigator must notify the Sponsor's Medical Monitor immediately. The Medical Monitor will determine if the subject may continue to receive IP.
Antimalarials:
   Chloroquine
   Hydroxychloroquine
   Quinacrine
AZA
Cyclosporine
   Cyclosporine eye drops are permitted for use while on study.
   Lifitegrast ophthalmic solution (Xiidra eye drops) are permitted for use while on study.
Danazol
Dapsone
GCs
   Intramuscular ≥80 mg/day methylprednisolone or equivalent.
   Intra-articular/tendon sheath/bursal injections of total methylprednisolone ≥80 mg or equivalent.
   IV≥40 mg/day but <1 g/day methylprednisolone or equivalent.
   OGC
      ≥40 mg/day prednisone or equivalent.
      Treatment above the OLE baseline (Day 1) dose for a dosing period of ≥14 days.
   Long biologic half-life (eg, dexamethasone, betamethasone).
   SC or intramuscular precursors (eg, ACTH).
Intra-articular medications other than GCs
Leflunomide
6-Mecaptopurine (6-MP)
MPA
MMF
MTX or any change in the route of administration of PO, SC, or intramuscular MTX
Sulfasalazine
Tacrolimus
Voclosporin

### Assessments

(1) Lupus Assessment Completion and Review Process. Appropriately trained and qualified Investigators will complete the lupus assessments, including but not limited to the Systemic Lupus Erythematosus Disease Activity Index 2000 (SLEDAI 2K), Cutaneous Lupus Erythematosus Disease Area and Severity Index (CLASI), and Joint Count. The SLEDAI-2K, Physician Global Assessment (PGA), and CLASI must be administered by the Investigator or appropriately qualified physician, unless prior Sponsor approval has been obtained for any other clinically trained site personnel with documentation of adequate assessment experience. The Joint Count evaluation can be completed by other site personnel who, as per Investigator discretion, are qualified to perform the assessments and have at least one year of experience administering the Joint Count evaluation.

(2) Systemic Lupus Erythematosus Disease Activity Index 2000. The SLEDAI-2K index consists of a list of organ manifestations, each with a definition. A certified Investigator or designated physician will complete the SLEDAI-2K assessment and decide whether each manifestation is “present” or “absent” within the last 4 weeks. The assessment also includes the collection of blood and urine for assessment of the laboratory categories of the SLEDAI-2K. The SLEDAI-2K assessment consists of 24 lupus-related items. It is a weighted instrument, in which descriptors are multiplied by an organ's “weight.” For example, renal descriptors are multiplied by 4 and central nervous system (CNS) descriptors by 8; these weighted organ manifestations are totaled into the final score. The SLEDAI-2K scores are valid, reliable, and sensitive clinical assessments of lupus disease activity. The SLEDAI-2K, which is calculated using a timeframe of 30 days prior to a visit for clinical and laboratory values, has been shown to be like the SLEDAI-2K with a 10 day window (Touma et al, 2010). A timeframe of 28 days (±allowed visit window) is used in this study.

(3) Modified SLEDAI-2K Flare Index. The classic Safety of Estrogens in Lupus Erythematosus National Assessment (SELENA)-Systemic Lupus Erythematosus Disease Activity Index (SLEDAI) Flare Index (SFI) is a composite outcome of the SELENA-SLEDAI, PGA (range 0-3), treatment modifications, and a few additional items (Arora et al, 2020; Petri et al, 2005). Thanou et al (2014) developed a modified version of the classic SFI that excludes the medication criteria from the classic SFI (Thanou et al, 2014; Thanou et al, 2018). In this OLE study, SLEDAI-2K are used instead of SELENA-SLEDAI and thus a modified SLEDAI 2K Flare Index are used, and scoring are as follows (28 day recall period): (A) Mild or moderate flare per expert clinician's opinion: (i) Change in SLEDAI-2K score of 3 points or more (but not to more than 12); (ii) New/Worse: Discoid, photosensitive, profundus, cutaneous vasculitis, bullous lupus, Nasopharyngeal ulcers, Pleuritis, Pericarditis, Arthritis, Fever (SLE), ≥1.0 increase in PGA score, but not to more than 2.5. (B) Severe flare: (i) Change in SLEDAI-2K score to greater than 12; (ii) New/worse: CNS-SLE, Vasculitis, Nephritis, Myositis, Platelets≤60,000, Hemolytic anemia: hemoglobin≤70 g/L or decrease in hemoglobin ≥30 g/L, Hospitalization for SLE Activity, Increase in PGA to ≥2.5.

(4) Physician Global Assessment. The PGA represents the physician's overall assessment of average SLE disease severity on a visual analog scale with 0 (no disease) to 3 (severe) disease activity over the previous 4 weeks. The PGA for a given subject should be completed by the same physician whenever possible. The PGA is a modification of the classic analog scale in that it is anchored with numbers from 0 to 3 demarcating no, mild, moderate, and severe disease. The number 3 indicates severe disease and is at the end of the scale. This refers to the most severe disease possible; it does not reflect the most severe disease observed in a particular subject, but the most severe disease ever observed in all SLE subjects. Therefore, the line made along this scale by the physician should virtually never approach this edge. Any disease rated greater than 2.5 is very severe. The range of moderate disease covers approximately 1.5 to 2.4. Mild disease falls below 1.5. The instrument is like a logarithmic scale, with greater distances or demarcations possible among more mild-moderate symptoms. When scoring the PGA, the score from the previous visit should be reviewed and the mark should be moved relative to the score from the previous visit. This is a global assessment, factoring in all aspects of the subject's lupus disease activity. It should not reflect non-lupus medical conditions.

(5) Lupus Low Disease Activity State. The Lupus Low Disease Activity State (LLDAS) is a composite measure of SLE disease activity that has been used in clinical studies (Franklyn et al, 2016). The LLDAS is defined and measured by attaining all the following 5 criteria: SLEDAI-2K≤4, with no activity in major organ systems (renal, CNS, cardiopulmonary, vasculitis, fever) and no hemolytic anemia or gastrointestinal activity, No new lupus disease activity compared with the previous assessment (SLEDAI 2K), PGA≤1 (scale 0 to 3), A current prednisone (or equivalent) dose≤7.5 mg daily, and well-tolerated standard maintenance doses of immunosuppressive drugs and approved biological agents.

(6) Oral Glucocorticoid Reduction.

(7) Systemic Lupus International Collaborating Clinics/American College Rheumatology Damage Index. The Systemic Lupus International Collaborating Clinics/American College of Rheumatology (SLICC/ACR) Damage Index (SDI) has been developed to assess irreversible damage in SLE subjects independently of its cause (SLE activity, therapy, comorbidities) but occurring after disease onset. Damage (ie, irreversible impairment since onset of SLE) is usually defined as a clinical feature that must be continuously present for at least 6 months to score. In addition, some irreversible events such as myocardial infarction or a cerebrovascular accident score as damage on their occurrence. Briefly, damage is defined for 12 organ systems: peripheral vascular, ocular, neuropsychiatric, renal, pulmonary, cardiovascular, gastrointestinal, musculoskeletal, skin, endocrine (diabetes), gonadal, and malignancies. Damage over time can be stable or increase, to a maximum of 47 points; however, there should be no decrease in the number of points.

(8) Cutaneous Lupus Erythematosus Disease Area and Severity Index. The CLASI is a validated index used for assessing the cutaneous lesions of SLE and consists of 2 separate scores: the first summarizes the inflammatory activity of the disease; the second is a measure of the damage done by the disease. The activity score considers erythema, scale/hypertrophy, mucus membrane lesions, recent hair loss, and nonscarring alopecia. The damage score represents dyspigmentation, scarring/atrophy/panniculitis, and scarring of the scalp. Subjects are asked if their dyspigmentation lasted 12 months or longer, in which case the dyspigmentation score is doubled. Each of the above parameters is measured in 13 different anatomical locations, included specifically because they are the most often involved in CLE. The most severe lesion in each area is measured.

(9) 28-Joint Count. The painful, swollen, and tender Joint Count is based on left and right shoulder, elbow, wrist, metacarpophalangeal (MCP)1, MCP2, MCP3, MCP4, MCP5, proximal interphalangeal (PIP)1, PIP2, PIP3, PIP4, and PIP5 joints of the upper extremities, and left and right knee of the lower extremities. Subjects are asked at the start of the Joint Count (prior to assessment of tenderness and swelling) whether they have experienced or are experiencing pain in any of the 28 joints within the last 30 days. An active joint for the SLEDAI-2K calculation is defined as a joint with pain and signs of inflammation (i.e., tenderness, swelling, or effusion). Each of the 28 joints will then be evaluated separately for tenderness (by palpating the joint) and swelling. Joints with intra-articular injection within 4 weeks are not evaluable for the assessment. The Joint Count assessment will include questions regarding limitation of range of movements and effects of joint symptoms on basic and functional activity of daily living.

(10) Safety Assessments are described in Table 5.

(11) Physical Examination. A full physical examination are performed at the Week 0 [Day 1] and Week 48 visits. A focused physical examination should be performed at all other visits. A focused physical examination should always include assessment of head, ears, eyes, nose, throat, lungs, heart, abdomen, skin, and extremities. Medically significant changes from the screening physical examination are recorded as AEs, unless they are considered a manifestation of SLE and captured on the SLEDAI 2K or CLASI.

(12) Vital Signs. Vital signs, blood pressure (mmHg), pulse rate (beats/min), respiratory rate (breaths/min), and body temperature (° C.), and body weight (kg) are obtained as outlined in Table 5 using clinically acceptable methods and devices as outlined therein. Vital signs should be measured in a seated position having rested in this position for at least 5 minutes before each reading and, when possible, should be taken before any blood draws. Prior to and after IP administration, vital signs should be checked as follows: Within 15 minutes prior to administration of IP but within 30 minutes on days when an ECG is to be performed. Every 30 minutes (±5 minutes) for 60 minutes after administration or until stable, whichever is later (for the first 2 study visits only). If anaphylaxis or a hypersensitivity reaction occurs after the SC administration of IP, vital signs are taken more frequently, based on Investigator's judgment and as warranted by the severity of the reaction.

(13) Electrocardiograms. A computerized 12-lead ECG are performed at the visits specified in Table 5. The Investigator or a qualified designee will review and indicate if the ECG is normal, abnormal but not clinically significant, or abnormal and potentially clinically significant. The ECG should be performed after vital signs are examined and after 10 minutes at rest in a supine position.

(14) Local Injection Tolerability Assessments. Daxdilimab are administered by study-site staff in the clinic, and each subject are assessed for local injection tolerability 60 minutes after the first and second doses (Day 1 and Week 12) are administered. The Investigator, or designee, will evaluate the injection sites at these visits, and will document the presence or absence of local intolerance/injection site reactions and will open an AE in case of local injection site intolerance. Additional local injection tolerability assessments are conducted at additional time points as specified in Table 5. Additional follow-up after Week 48 can be performed for any ongoing injection site reactions.

(15) Clinical Safety Laboratory Tests. All protocol-required laboratory tests, as defined in Table 5, must be conducted in accordance with the laboratory manual and at a central laboratory at the visits specified in Table 5. Abnormal laboratory findings associated with the underlying disease are not considered clinically significant unless judged by the Investigator to be more severe than expected for the subject's condition. All laboratory tests with values considered clinically significantly abnormal during participation in the study should be repeated as soon as possible (preferably within 24 to 48 hours) until the values return to normal or baseline or are no longer considered clinically significant by the Investigator or Medical Monitor. Exemplary clinical laboratory tests are shown in Table 6.

TABLE 6
Clinical Laboratory Tests
Immunology

| | |
|---|---|
| ANA | Anti-SSA |
| Anti- dsDNA | Anti-SSB |
| Anti-Smith | C3 and C4 |
| Anti-RNP | Rheumatoid factor |

Hematology and coagulation

| | |
|---|---|
| Hemoglobin | PT/INR and PTT |
| Hematocrit | RBC count |
| Mean corpuscular hemoglobin concentration | Reticulocyte count |
| Mean corpuscular volume | WBC with differential |
| Platelet count | |

Pregnancy tests

| | |
|---|---|
| Urine dipstick β-hCG | FSH |

Other laboratory tests

| | |
|---|---|
| HbA1c | pDC |
| Hs-CRP | Serum IgG, IgA, and IgM |
| Lipids (TC, TG, HDL-C, and LDL-C) | |

Serum chemistry

| | |
|---|---|
| Albumin | Creatinine |
| ALT | eGFR |
| ALP | GGT |
| AST | Glucose |
| Bicarbonate | Potassium |
| Blood urea nitrogen | Sodium |
| Calcium | Total bilirubin |
| Chloride | Total protein |
| Creatine kinase | |

Serum virology
HBcAb (reflex DNA testing if isolated hepatitis B core positive)
Urinalysis

| | |
|---|---|
| Dipstick | Leukocyte esterase |
| Appearance | pH |
| Bilirubin | Protein |
| Blood | Specific gravity |
| Color | Microscopy, including WBC/HPF, RBC/HPF, and casts |
| Epithelial cells | Urine creatinine and protein; spot UPCr |
| Glucose | |
| Ketones | |

ALP = alkaline phosphatase; ALT = alanine aminotransferase; ANA = antinuclear antibodies; AST = aspartate aminotransferase; β-hCG = beta-human chorionic gonadotropin; C = complement; dsDNA = double-stranded DNA; eGFR = estimated glomerular filtration rate; FSH = follicle-stimulating hormone; GGT = gamma glutamyl transferase; HbA1c = glycosylated hemoglobin; HBcAb = hepatitis B core antibody; HDL-C = high-density lipoprotein-cholesterol; HIV = human immunodeficiency virus; HPF = high-power field; hs-CRP = high-sensitivity C-reactive protein; Ig = immunoglobulin; INR = international normalized ratio; LDL-C = low-density lipoprotein-cholesterol; pDC = plasmacytoid dendritic cell; PT = prothrombin time; PTT = partial thromboplastin time; RBC = red blood cell; RNP = ribonucleoprotein; SSA = Sjogren's syndrome-related antibody A; SSB = Sjogren's syndrome-related antibody B; TC = total cholesterol; TG = triglycerides; UPCr = urine protein:creatinine ratio; WBC = white blood cell

Hepatitis B DNA testing (Reflex DNA testing) will continue to be performed Q12W if the subject had an isolated hepatitis B core positive result in the RECAST SLE study. The IP are discontinued if the subject's hepatitis B virus DNA levels are confirmed to exceed the lower limit of quantitation as per the central laboratory.

Pharmacokinetics. Serum for PK analysis are collected at the visits specified in Table 4 and analyzed using a validated bioanalytical method.

Pharmacodynamics. Whole blood are collected for the assessment of pDC levels by flow cytometry at the visits specified in Table 4. Daxdilimab binds to ILT7 on the surface of pDCs leading to recruitment of macrophages and NK cells, thus inducing apoptosis and reduction in the number of pDCs.

Biomarker Evaluation. Whole blood are collected and processed into serum, plasma, and peripheral blood mononuclear cells (PBMCs) at time points specified in Table 4. Samples are collected for evaluation of IFNα (serum) and myxovirus resistance protein A (whole blood). The following additional samples are collected and assessed for exploratory biomarkers related to the mechanism of action of daxdilimab or SLE disease activity: serum (e.g., autoantibody profiles, cytokine/chemokine profiles, and other proteins or factors related to SLE disease pathways), plasma (e.g., complement breakdown products), whole blood transcriptomics (e.g., IFN gene signature, or genes associated with disease activity), PBMCs (e.g., frequencies and/or gene expression of immune cell populations).

Immunogenicity Assessments. Serum for immunogenicity ADA analysis is collected at the visits specified in Table 4 and assessed using a validated immunoassay. Serum samples are screened for antibodies binding to daxdilimab and the titer of confirmed positive samples are reported. Other analyses may be performed to further characterize the immunogenicity of daxdilimab. Antibodies may be further characterized and/or evaluated for their ability to neutralize the activity of daxdilimab.

(16) Pregnancy Testing. Urine pregnancy tests are performed in women of childbearing potential during treatment and follow-up at the study site using a dipstick, as indicated in Table 5.

(17) Quality of Life Assessment. Quality of life and pharmacoeconomic assessments are made at the visits specified in Table 5.

(18) Patient Global Assessment. Subjects complete the Patient Global Assessment (PtGA). The PtGA is a single-item question that considers all the ways in which illness and health conditions may affect the subject at this time. The subject should consider the previous week when answering this question. Responses range from “very well” to “very poor” on a 100 mm visual analog scale.

(19) Patient Global Impression of Change. The Patient Global Impression of Change is self-rated scale that asks the subject to describe the change in activity limitations, symptoms, emotions, and overall quality of life related to the subject's condition on the following scale: 1 (very much improved), 2 (much improved), 3 (minimally improved), 4 (no change), 5 (minimally worse), 6 (much worse), and 7 (very much worse).

(20) Lupus Quality of Life. Lupus Quality of Life (LupusQoL) is a 34-item SLE-specific health-related quality of life measure. The instrument consists of 8 domains (physical health [8 items], pain [3 items], planning [3 items], intimate relationships [2 items], burden to others [3 items], emotional health [6 items], body image [5 items], and fatigue [4 items]).

### Example 3: A Phase 2a, Open Label, Proof-of-Concept Trial of Daxdilimab for the Treatment of Moderate-to-Severe Alopecia Areata

This is a Phase 2a, multicenter, open-label, proof-of-concept trial to assess the preliminary efficacy, safety, tolerability, PK, and PD of Daxdilimab in subjects with moderate-to-severe AA, with ≥50% and ≤95% total scalp hair loss as defined by the SALT score at Screening and Day 1. Subjects are between 18 and 65 years of age (inclusive), with a current episode of hair loss of at least about 3 months but less than 7 years, along with investigator's assessment that hair regrowth is possible and no evidence of active regrowth present at baseline, and no known history of significant regrowth during the last 6 months. Approximately 30 subjects are enrolled to receive Daxdilimab 300 mg administered subcutaneously Q4W for 32 weeks. The trial will comprise of a screening period of up to 30 days with enrollment on Day 1. Subjects are treated with Daxdilimab 300 mg Q4W through Week 32. The primary endpoint assessment will occur during the week 24 visit. At week 24, the effect of treatment on reducing hair loss are evaluated. Also evaluated are long-term safety and the duration of efficacy following the treatment period. Subjects who prematurely stop dosing are followed through Week 48 unless subjects withdraw consent of trial participation or are lost to follow-up. Any subject who discontinues from the trial prematurely and does not plan to participate in the post-treatment follow-up period are requested to complete the early termination (ET) visit.

For scheduled trial visits, subjects will come to the trial centers on 14 occasions: screening, Day 1, Weeks 4, 8, 12, 16, 20, 24, 28, 32, 36, 40, 44, and 48 (early termination [ET]).

Efficacy are evaluated by assessment of SALT scores, ALODEX scores, ClinRO Measure for Eyebrow Hair Loss™, and ClinRO Measure for Eyelash Hair Loss™. Photographs of the full scalp for all subjects, and of the eyebrows and eyelashes for subjects who have hair loss in these areas at baseline, will also be taken to assess changes in hair growth during the trial.

Blood samples are collected from all subjects to characterize the PK, PD, and immunogenicity of Daxdilimab.

Safety are assessed by collecting AEs, SAEs, AESIs, performing local injection site tolerability assessment, recording vital signs, performing physical examinations and ECGs, and evaluating clinical laboratory results

A total of up to 30 subjects will receive Daxdilimab in the trial. The sample size was determined by the need to evaluate potential efficacy, safety and tolerability, and PK/PD. An exemplary trial diagram is shown in FIG. 1.

### Treatment

The study involves SC administration of Daxdilimab 300 mg (two 1.5 mL injections) Q4W for 32 weeks. On Day 1 and Week 4, subjects will remain under observation for at least 1 hour after IP administration. The IP are provided by the sponsor. Further details are found in Table 7.

TABLE 7
Treatment

| | Product |
|---|---|
| Product name | Daxdilimab |
| Unit dose strength(s)/ | 300 mg dose, administered as 2 × 1.5 |
| Dosage level(s) | mL SC injections. |
| Route of | Subcutaneous, Q4W for 32 weeks (total |
| Administration | of 9 doses) |
| Physical description | The drug product are provided as a sterile, |
| | white to off-white, lyophilized powder. |
| Dosing instructions | Daxdilimab should be administered by clinic staff |
| | trained in best practices for SC administration of |
| | treatments. More details on the administration |
| | method are described in the study manual. |

Abbreviations: Q4W, once every 4 weeks; SC, subcutaneous.

### Inclusion Criteria

All subjects must meet all of the following criteria to be eligible for trial participation, either at the screening and Day 1 visits, or only at one of the specified visits (screening or Day 1) as noted in the criterion:
1. Written informed consent and any locally required authorization (e.g., Health Insurance Portability and Accountability Act in the United States) obtained from the subject prior to performing any protocol-related procedures, including screening evaluations.
2. Adult men and women aged 18 to 65 years, inclusive, at the time of informed consent.
3. Willing and able to comply with the prescribed treatment protocol and evaluations for the duration of the trial.
4. Willing to keep the same hair style and color (e.g., hair products, process, and timing for hair appointments) for the duration of the trial.
5. Clinical diagnosis of moderate-to-severe AA-defined as the presence of ≥50% and ≤95% total scalp hair loss at screening and baseline (Day 1) defined by the SALT score.
6. Duration of current episode of hair loss over 3 months but less than 7 years at screening and Day 1, along with investigators' assessment that hair regrowth is possible. Total duration since diagnosis of AA could be ≥7 years.
7. No evidence of active regrowth present at baseline and no known history of significant regrowth, as per investigator's judgement, over the last 6 months.
8. Female of childbearing potential must have a negative serum pregnancy test at screening and negative urine pregnancy test at Day 1.
9. For female subject of childbearing potential involved in any sexual intercourse that could lead to pregnancy: the subject must agree to use a highly effective contraceptive method from at least 4 weeks prior to Day 1 until at least 6 months (approximately 5 half-lives) after the last IP administration or the end of the trial, whichever is longer, and refrain from egg retrieval/egg donation during this period. Highly effective contraceptive methods include hormonal contraceptives (e.g., combined oral contraceptive, patch, vaginal ring, injectable, or implant), intrauterine devices or intrauterine systems, vasectomized partner(s) (provided his vasectomy was performed ≥4 months prior to Screening), tubal ligation or double barrier methods of contraception (e.g., male condom with cervical cap, male condom with diaphragm, and male condom with contraceptive sponge) in conjunction with spermicide.
   Note: For countries where double barrier methods are not accepted as highly effective contraception, this option must not be considered.
   Note: Subjects must have been on a stable dose of hormonal contraceptives for at least 4 weeks before Day 1.
   Note: The above list of contraceptive methods does not apply to subjects who are abstinent for at least 4 weeks before Day 1 and will continue to be abstinent from penile-vaginal intercourse throughout the trial and for at least 6 months after the last IP administration or until the end of the trial, whichever is longer. The reliability of sexual abstinence needs to be evaluated in relation to the duration of the clinical trial and the preferred and usual lifestyle of the subject. Periodic abstinence (calendar, symptothermal, post-ovulation methods) is not acceptable.
   Note: A female subject of nonchildbearing potential is defined as follows:
      Female subject who has had surgical sterilization (hysterectomy, bilateral oophorectomy, or bilateral salpingectomy).
      Female subject who has had a cessation of menses for at least 12 months prior to the screening visit without an alternative medical cause, and a follicle-stimulating hormone (FSH) test confirming nonchildbearing potential (refer to laboratory reference ranges for confirmatory levels).
   10. For male subject involved in any sexual intercourse that could lead to pregnancy, subject must agree to use one of the highly effective contraceptive methods listed in Inclusion Criterion #9, from Day 1 until at least 6 months (approximately 5 half-lives) after the last IP administration and refrain from donating sperm during this period. If the female partner of a male subject uses any of the hormonal contraceptive methods listed above, this contraceptive method should be used by the female partner from at least 4 weeks before Day 1 until at least 6 months after the last administration of Daxdilimab.

### Exclusion Criteria:

Subjects are ineligible for trial participation if they meet any of the following criteria at the Screening and/or Day 1 visits, as applicable:

(1) Individuals involved in the conduct of the trial, their employees, or immediate family members of such individuals. (2) Any clinically significant medical condition or physical/laboratory/ECG/vital signs abnormality that would, in the opinion of the investigator, put the subject at undue risk or interfere with the evaluation of the IP or interpretation of trial results. (3) History of allergy, hypersensitivity reaction, or anaphylaxis to any component of the IP or to a previous monoclonal antibody (mAb) or human immunoglobulin (Ig) therapy. (4) Subject has had excessive sun exposure, is planning a trip to a sunny climate, or has used tanning booths within 4 weeks prior to Day 1 or is not willing to minimize natural and artificial sunlight exposure during the trial. Use of sunscreen products and protective apparel are recommended when sun exposure cannot be avoided. (5) Spontaneous or induced abortion or still or live birth≤4 weeks prior to Screening. (6) Breastfeeding or pregnant women or women who intend to become pregnant anytime from signing the ICF through 6 months after receiving the last dose of IP or until the end of the trial, whichever is longer. (7) History of drug or alcohol abuse that, in the opinion of the investigator, might affect subject safety or compliance with visits, or interfere with other trial assessments. (8) Major surgery within 8 weeks prior to Screening or elective surgery planned during the trial. (9) Known history of a primary immunodeficiency or an underlying condition such as known human immunodeficiency virus (HIV) infection, a positive result for HIV infection, splenectomy, or any underlying condition that in the opinion of the investigator significantly predisposes the subject to infection. (10) At Screening, any of the following (tests may be repeated once within the same Screening period to confirm results prior to Day 1):
Aspartate aminotransferase (AST)≥2.5×upper limit of normal (ULN)
Alanine aminotransferase (ALT)≥2.5×ULN
Total bilirubin≥1.5×ULN (unless due to Gilbert's syndrome)
Serum immunoglobulin G (IgG)≤600 mg/dL (or ≤6 g/L)
Neutrophil count≤1000/μL (or ≤1.0×10⁹/L)
Platelet count≤50,000/μL (or ≤50×10⁹/L)
Hemoglobin≤8 g/dL (or ≤80 g/L)
Total lymphocyte count≤200 cells/mm³
Glomerular filtration rate (Modification of Diet in Renal Disease [MDRD])≤30 mL/min/1.73 m²
Spot urine protein to creatinine ratio (UPCr)≥3 mg/mg

(11) Confirmed positive test for hepatitis B serology defined as: Hepatitis B surface antigen (HBsAg), or Hepatitis B core antibody (HBcAb or anti-HBc). (12) Positive test for hepatitis C virus antibody. (13) Active tuberculosis (TB), or a positive TB test at Screening. Subject are evaluated for latent TB infection with a purified protein derivative (PPD) test or a QuantiFERON-TB Gold test. Subjects who demonstrate evidence of latent TB infection (either PPD≥5 mm of induration or positive QuantiFERON-TB Gold test, irrespective of bacille Calmette-Guérin vaccination status will not be allowed to participate in the trial, unless documented history of appropriate treatment for active or latent TB. Subjects with an indeterminate test result can repeat the test, but if the repeat test is also indeterminate, they are excluded. (14) Any severe herpes virus family infection (including Epstein-Barr virus, cytomegalovirus [CMV]) at any time prior to Day 1, including, but not limited to, disseminated herpes, herpes encephalitis, recent recurrent herpes zoster (defined as 2 episodes within the last 2 years), or ophthalmic herpes. (15) Any herpes zoster, CMV, or Epstein-Barr virus infection that was not completely resolved 12 weeks prior to Day 1. (16) Any of the following within 30 days prior to signing the ICF and though Day 1:
Clinically significant active infection in the opinion of the investigator, including ongoing, and chronic infection requiring antibiotics or antiviral medication (chronic nail infections are allowed). Note: Subject with a limited recurrence of a cold sore or herpes genitalis between ICF signature and Day 1 could be eligible based on the investigator's judgement.
Any infection requiring hospitalization or treatment with intravenous (IV) anti-infectives.
A subject with a documented positive severe acute respiratory syndrome coronavirus 2 (SARS-COV-2) test may be rescreened at least 2 weeks after a positive test if the subject is asymptomatic and at least 3 weeks after resolution of symptomatic Coronavirus Disease 2019 (COVID-19) illness.

(17) Opportunistic infection requiring hospitalization or parenteral antimicrobial treatment within 2 years prior to Day 1. (18) Any acute illness or evidence of clinically significant active infection, such as fever ≥38.0° C. (≥100.5° F.) on Day 1. (19) History of clinically significant cardiac disease including unstable angina; myocardial infarction within 6 months prior to Day 1; congestive heart failure; arrhythmia requiring active therapy, except for clinically insignificant extra systoles, or minor conduction abnormalities; or presence of clinically significant abnormality on ECG if, in the opinion of the investigator, it would increase the risk of trial participation. (20) History of cancer or lymphoproliferative disease within 5 years prior to Day 1, except as follows:
In situ carcinoma of the cervix treated with apparent success with curative therapy ≥12 months prior to Screening, or
Nonmetastatic cutaneous basal cell or squamous cell carcinoma of the skin treated with apparent success with curative therapy.

(21) Receipt of a live or live-attenuated vaccine within 8 weeks prior to Day 1 or plans to receive a live or live attenuated vaccine during the trial and up to 4 weeks after the last IP administration. (22) Nonlive and nonlive-attenuated vaccines for COVID-19 (e.g., RNA-based vaccines, protein-based vaccines, and nonreplicating viral vector-based vaccines) are not allowed within 4 weeks prior to Day 1 and within 2 weeks prior to each trial visit. (23) Subject should be assessed for epidemiologic risk of COVID-19 (i.e., recent exposure, high-risk housing) and for health-related risk of COVID-19 severity based on current understanding of risk factors for severe disease when making a decision regarding the individual's risk of participation. Subjects who have COVID-19 or other significant infection, or in the judgment of the investigator, may be at a high risk of COVID-19 or its complications should not be enrolled. (24) Subject who has given ≥50 ml of blood or plasma within 30 days of Screening or ≥499 mL of blood or plasma within 56 days of Screening (during a clinical trial or at a blood bank donation) or plans to give blood or plasma during their participation in the trial or up to 6 months after the last IP administration, whichever is longer. (25) Transfusion with blood, packed red blood cells, platelets or treatment with plasmapheresis, or plasma exchange within 8 weeks prior to Day 1 and for the total duration of the trial participation.

### Disease-Related Exclusion Criteria

(26) Active forms of other inflammatory skin disease(s) or evidence of other skin conditions (e.g., psoriasis, seborrheic dermatitis, lupus) at the time of the Screening and through Day 1, that in the opinion of the investigator might interfere with evaluation of AA and the assessment of the activity measures. (27) Presence of another form of alopecia (except for androgenic alopecia). (28) History of male or female pattern hair loss≥Hamilton stage III or ≥Ludwig stage II. (29) History or presence of hair transplants. (30) History or presence of micropigmentation of the scalp (Note: microblading of the eyebrows is permitted).

### Prior and Concomitant Therapy Criteria

(31) Use of steroids (systemic and intralesional), anthralin, squaric acid, diphenylcycloprophenone (DPCP), dinitrochlorobenzene (DCNB), protopic, minoxidil, or any other medication which in the opinion of the investigator may affect hair regrowth within 4 weeks of Day 1 visit. Intranasal and inhaled corticosteroids are allowed, eye and ear drops containing corticosteroids are also allowed. (32) Use of platelet-rich plasma injections in the last 12 weeks prior to Day 1. (33) Topical medicated treatment that could affect AA including, but not limited to, topical corticosteroids, calcineurin inhibitors, antimicrobials, medical devices within 2 weeks of Day 1 visit. Topical corticosteroids are permitted outside of the scalp, eyebrows, and eyelids. (34) Subjects who have had previous treatment with any biologic B-cell-depleting therapy (e.g., rituximab, ocrelizumab, or ofatumumab) or other B-cell targeting therapy (e.g., belimumab) within 12 months before Day 1. (35) Subjects who have received previous treatment with pDC inhibiting therapies (e.g., anti-ILT7, anti-blood dendritic cell antigen 2 [BDCA2]). (36) Inadequate response to adequate trial of oral Janus Kinase (JAK) inhibitors. Previous exposure to topical JAK inhibitors is acceptable, regardless of response. (37) Any biologic or conventional disease-modifying antirheumatic drugs (DMARD), immunosuppressant (e.g., cyclosporine, methotrexate, or azathioprine), JAK-inhibitors, interferon (IFN) blocking therapies, or antiproliferative agents, if last dose was taken: a. within 8 weeks prior to Day 1 or b. drug-specific 5 half-lives elimination period (if longer than 8 weeks). (38) Subject has received any marketed or investigational biological agent within 12 weeks or 5 half-lives (whichever is longer) prior to Day 1. (39) Currently receiving a nonbiological IP or device or has received one within 4 weeks prior to Day 1 or within 5 published half-lives, whichever is longer. (40) Subject has received any ultraviolet (UV)-B phototherapy (including tanning beds), has had psoralen-UV-A (PUVA) treatment, or excimer laser within 4 weeks prior to Day 1.

### Concomitant Therapy

All medications (including over-the-counter drugs, vitamins, herbal/natural products, and antacids) taken within 4 weeks prior to screening and throughout the trial must be recorded. In addition, the use of any prohibited medications must be recorded within the timeframe described in the exclusion criteria. No rescue medications or treatments are permitted in this study.

### Permitted Therapies

The following therapies are permitted: Intranasal corticosteroids and inhaled corticosteroids, Eye and ear drops containing corticosteroids, sunscreen products and protective apparel are permitted when sun exposure cannot be avoided, topical corticosteroids are permitted outside of the scalp, eyebrows, and eyelids, nonlive and nonlive-attenuated vaccines for COVID-19 (e.g., RNA-based vaccines, protein-based vaccines, and nonreplicating viral vector-based vaccines) are allowed ≥4 weeks prior to Day 1 and ≥2 weeks prior to each trial visit.

### Prohibited Therapies or Procedures

Prohibited therapies or procedures are shown in Table 8.

TABLE 8
Summary of prohibited medications, products, and procedures

| | Washout period prior to |
|---|---|
| Prohibited medications, products, and procedures | first dose (Day 1) |
| Any pDC inhibiting therapies (e.g., anti-ILT7, anti-BDCA2). | Lifetime |
| History or presence of hair transplants. | Lifetime |
| Micropigmentation of the scalp (Note: microblading of the eyebrows | Lifetime |
| is permitted). | |
| Any biologic B-cell-depleting therapy (e.g., rituximab, ocrelizumab, or | 12 months |
| ofatumumab) or other B-cell targeting therapy (e.g., belimumab). | |
| Any marketed or investigational biological agent. | 12 weeks or 5 half-lives, |
| | whichever is longer |
| Platelet-rich plasma injections. | 12 weeks |
| Transfusion with blood products, packed red blood cells, platelets or | 8 weeks |
| treatment with plasmapheresis, or plasma exchange. | |
| Any biologic or conventional DMARD, immunosuppressant (e.g., | 8 weeks or 5 half-lives |
| cyclosporine, methotrexate, or azathioprine), JAK-inhibitor, IFN | whichever is longer |
| blocking therapies, or antiproliferative agents. | |
| Live and live attenuated vaccines. | 8 weeks |
| Nonbiological investigational product or device. | 4 weeks or 5 published |
| | half-lives, whichever is |
| | longer |
| Nonlive and nonlive-attenuated vaccines for COVID-19 (e.g., RNA- | 4 weeks prior to Day 1 |
| based vaccines, protein-based vaccines, and nonreplicating viral | and within 2 weeks |
| vector-based vaccines). | prior to each trial visit |
| PUVA treatment, UV-B phototherapy (including tanning beds) or | 4 weeks |
| excimer laser, excessive sun exposure or has used tanning booths. | |
| Steroids (systemic and intralesional), anthralin, squaric acid, DPCP, | 4 weeks |
| DCNB, protopic, minoxidil, or any other medication which in the | |
| opinion of the investigator may affect hair regrowth. Intranasal and | |
| inhaled corticosteroids are allowed, eye and ear drops containing | |
| corticosteroids are also allowed. | |
| Topical medicated treatment that could affect AA including, but not | 2 weeks |
| limited to, topical corticosteroids, calcineurin inhibitors, | |
| antimicrobials, medical devices. Topical corticosteroids are permitted | |
| outside of the scalp, eyebrows, and eyelids. | |

### Efficacy Analysis

The percent change from baseline in SALT score at Week 24 are summarized with descriptive statistics. All other efficacy endpoints are analyzed descriptively, with summary statistics for continuous endpoints and frequency distribution for binary endpoints.

### Safety Analysis

The occurrence of treatment-emergent adverse events (TEAEs), treatment-emergent serious adverse events (TESAEs), and adverse events of special interest AESIs are summarized by Medical Dictionary for Regulatory Activities (MedDRA) System Organ Class (SOC) and Preferred Term (PT), as well as by severity and by relationship to the IP. Other safety parameters, including but not limiting to laboratory assessments and vital signs will also be summarized as appropriate.

### Pharmacodynamic and Biomarker Analyses

The observed count along with the change and percent change from baseline are summarized descriptively as appropriate.

### Pharmacokinetic Analysis

Serum concentrations are summarized descriptively by visit. Additional PK analyses may be conducted as appropriate and reported separately from the clinical study report.

### Anti-Drug-Antibodies

Anti-drug-antibody (ADA) rate are determined.

### Planned Analyses

The primary analysis are performed when the last subject has completed Week 24 or withdraws prior to the scheduled Week 24 visit. All available data at the time of the data cut-off (including data collected after Week 24) are included in the primary analysis. The final analysis are performed when all subjects have completed trial.

### Schedule of Assessments

A description of the procedures to be performed at each visit are depicted in Table 9. Unless specified otherwise, the assessments scheduled on Day 1 must be performed before the IP administration. The recommended order for performing the trial assessments is as follows, except for collection of blood samples for analysis of serum Daxdilimab ADA and PK samples, which must be performed prior to IP administration at the visits specified in Table 9: Investigator assessments, vital signs, 12-lead ECG, medical photographs, and blood draw.

Clinical evaluations of AA are performed by an experienced and qualified dermatologist (board certified or equivalent) or other suitably qualified and experienced designee.

TABLE 9
Schedule of assessments of Example 3

| | Procedure | | | | | | | | | | | | | | |
|---|---|---|---|---|---|---|---|---|---|---|---|---|---|---|---|
| | | Treatment Period | | | | | | | | | | | | | |
| | | | | | | | | | | Wk. | Post-Treatment Follow-Up | | | | |
| | | Day | Wk. | Wk. | Wk. | Wk. | Wk. | Wk. | Wk. | 32 | Wk. | Wk. | Wk. | Wk. | |
| | Screening | 1 | 4 | 8 | 12 | 16 | 20 | 24 | 28 | (EOT) | 36 | 40 | 44 | 48 | ET¹ |
| | Window (days) | | | | | | | | | | | | | | |
| | (Day −30 | | | | | | | | | | | | | | |
| | to −1) | — | ±3 | ±3 | ±3 | ±3 | ±3 | ±3 | ±3 | ±3 | ±3 | ±3 | ±3 | ±3 | — |
| Informed consent | X | | | | | | | | | | | | | | |
| Demographics | X | | | | | | | | | | | | | | |
| Medical and surgical history | X | X | | | | | | | | | | | | | |
| Inclusion/exclusion criteria | X | X | | | | | | | | | | | | | |
| Height | X | | | | | | | | | | | | | | |
| Body weight | X | | | | X | | | X | | | X | | | X | X |
| Physical examination² | X | X | X | X | X | X | X | X | X | X | X | X | X | X | X |
| Vital signs₃ | X | X | X | X | X | X | X | X | X | X | X | X | X | X | X |
| Clinical laboratory tests | X | X | X | X | X | X | X | X | X | X | X | X | X | X | X |
| (biochemistry, hematology, | | | | | | | | | | | | | | | |
| and urinalysis)⁴ | | | | | | | | | | | | | | | |
| Serology testing (HIV, HCV, | X | | | | | | | | | | | | | | |
| and HBV [HBsAg, Hbcp or | | | | | | | | | | | | | | | |
| anti-HBc]) | | | | | | | | | | | | | | | |
| 12-lead ECG⁵ | X | X | | | | | | X | | | | | | X | X |
| Pregnancy testing⁶ | X | X | X | X | X | X | X | X | X | X | X | X | X | X | X |
| TB evaluation⁷ | X | | | | | | | | | | | | | | |
| SALT assessment | X | X | X | X | X | X | X | X | X | X | X | X | X | X | X |
| ALODEX assessment | | X | X | X | X | X | X | X | X | | X | X | X | X | X |
| Medical photography⁸ | X | X | X | X | X | X | X | X | X | X | X | X | X | X | X |
| Eyelash/Eyebrow | X | X | X | X | X | X | X | X | X | X | X | X | X | X | X |
| Assessment (ClinRO) | | | | | | | | | | | | | | | |
| Whole blood | | X | X | | X | | | X | | X | X | | | X | X |
| transcriptomics | | | | | | | | | | | | | | | |
| DC flow cytometry | X | X | X | | X | | | X | | X | X | | | X | X |
| PBMCs | | X | X | | | | | X | | X | | | | | |
| Serum biomarkers | X | X | X | | X | | | X | | X | X | | | X | X |
| Plasma biomarkers | X | X | X | | X | | | X | | X | X | | | X | X |
| Blood MX | | X | X | | X | | | X | | X | X | | | X | X |
| Daxdilimab administration⁹ | | X | X | X | X | X | X | X | X | X | | | | | |
| Daxdilimab serum PK | | X | X | X | X | X | X | X | X | X | X | X | X | X | X |
| testing¹⁰ | | | | | | | | | | | | | | | |
| Serum Daxdilimab ADA¹¹ | | X | X | X | X | | | X | | | X | | | X | X |
| Local injection tolerability¹² | | | X | X | X | X | X | X | X | X | X | | | | |
| Concomitant medication | Continuous | | | | | | | | | | | | | | |
| monitoring | | | | | | | | | | | | | | | |
| Adverse event monitoring | Continuous | | | | | | | | | | | | | | |
| (AEs, SAEs, and/or AESIs) | | | | | | | | | | | | | | | |

Abbreviations: ADA, anti-drug antibodies; AE, adverse event; AESI, adverse event of special interest; anti-HBC, antibody to hepatitis B core antigen; ALODEX, Alopecia Density and Extent; HBsAg, Hepatitis B surface antigen; ClinRO, Clinician Reported Outcome; DC, dendritic cell; ECG, electrocardiogram; EOT, end of treatment; ET, early termination; HBcAb, hepatitis B core antibody; HBV, hepatitis B virus; HCV, hepatitis C virus; HIV, human immunodeficiency virus; IFN, interferon; IP, investigational product (Daxdilimab); MxA, myoxvirus protein A; PBMCs, peripheral blood mononuclear cells; PK, pharmacokinetic; PPD, purified protein derivative; Q4 W, every 4 weeks; SAE, serious adverse event; SALT, Severity of Alopecia Tool; SC, subcutaneous; TB, tuberculosis; Wk., week; WOCBP, women of childbearing potential.
¹Subjects who prematurely stop dosing will enter the post-treatment follow-up period of 16 weeks after the last dose received. Subjects who do not enter the post-treatment follow-up period are requested to complete the ET visit.
²A focused physical examination is performed.
³Vital signs comprise heart rate, blood pressure, temperature, and respiratory rate. Vital signs are performed pre-dose during the Treatment Period.
⁴Laboratory tests are performed pre-dose during the Treatment Period.
⁵12-lead ECG to be done pre-dose during the Treatment Period.
⁶WOCBP will have a serum pregnancy test at screening and urine pregnancy tests at other visits.
⁷The results of an IFN-gamma release assay (i.e., QuantiFERON-TB Gold performed within 12 weeks of the Screening visit (if available) are acceptable, provided there is no reason to suspect any re-exposure. If PPD is used, a second visit are necessary for PPD reading only.
⁸Medical photographs of the full scalp are taken for all subjects, and of the eyebrows and eyelashes for subjects who have hair loss in these areas at baseline.
⁹Subjects will receive Daxdilimab 300 mg administered SC on Day 1 followed by Q4 W through Week 32. All IP administrations are done at the clinical site bythe trial staff. On Day 1 and Week 4, subjects will remain under observation for at least 1 hour after IP administration.
¹⁰Serum Daxdilimab PK are obtained on Day 1 at the following timepoint: 2 hours (±5 minutes) post-dose. Samples for PK are obtained pre-dose on Weeks 4-32, and any time during the trial visit on Weeks 36-48.
¹¹Collection of blood samples for analysis of serum Daxdilimab ADA must be performed prior to IP administration at all applicable visits.
¹²Local injection tolerability assessments including injection site reactions are performed approximately 30 minutes post-injection (can be performed any time at Week 36 as no Daxdilimab is administered). Additional follow-up after Week 36 can be performed for any ongoing injection site reactions.

Efficacy are evaluated by assessment of SALT scores, ALODEX scores, Clinician Reported Outcome (ClinRO) Measure for Eyebrow Hair Loss™, and ClinRO Measure for Eyelash Hair Loss™. Photographs of the full scalp for all subjects, and of the eyebrows and eyelashes for subjects who have hair loss in these areas at baseline, will also be taken to assess changes in hair growth during the trial.

Blood samples are collected from all subjects to characterize the PK, PD, and immunogenicity of Daxdilimab.

Objectives and endpoints of the study are provided in Table 10.

TABLE 10
Objectives and endpoints.

| Primary Objective | Efficacy Endpoint |
|---|---|
| To evaluate the effect of | Percent change from baseline in SALT score at Week 24. |
| Daxdilimab on reducing hair | |
| loss at Week 24 in subjects | |
| with AA. | |
| Secondary Objectives | Efficacy Endpoints |
| To evaluate the effect of | Percent change from baseline in SALT score at Weeks 12- |
| Daxdilimab on reducing hair | 20; 28-36. |
| loss through 9 months of | Proportion of subjects who achieve ≥50% reduction in |
| treatment in subjects with | SALT from baseline at Weeks 12-36. |
| AA. | Proportion of subjects with absolute SALT score ≤ 10, 20, |
| | 30, 50 at Weeks 12-36. |
| To evaluate the post- | Percent change from baseline in SALT score at Weeks |
| treatment duration effect of | 40-48. |
| Daxdilimab on reducing hair | Proportion of subjects who achieve ≥50% reduction in |
| loss in subjects with AA. | SALT from baseline at Weeks 40-48. |
| | Proportion of subjects with absolute SALT score ≤ 10, 20, |
| | 30, 50 at Weeks 40-48. |
| PK/PD/Immunogenicity | PK/PD/Immunogenicity Endpoints |
| Objectives | |
| To characterize the PK, PD, | Daxdilimab concentrations. |
| and immunogenicity of | Change from baseline in pDCs. |
| Daxdilimab. | ADA rate. |
| Safety Objectives | Safety Endpoints |
| To evaluate the safety and | Incidence of AEs, SAEs, and AESIs. |
| tolerability of Daxdilimab. | |
| Exploratory Objectives | Efficacy Endpoints |
| To evaluate the effect of | Change from baseline in ClinRO for Eyebrow Hair Loss at |
| Daxdilimab on reducing | each visit. |
| eyelash and eyebrow loss in | Change from baseline in ClinRO for Eyelash Hair Loss at |
| subjects with AA. | each visit. |
| | Proportion of subjects achieving ClinRO for Eyebrow Hair |
| | Loss scores of 0 or 1 (full coverage or minimal gaps) with a |
| | ≥2-point improvement from baseline at each visit (among |
| | those with baseline scores ≥2 [significant gaps to no notable |
| | hair]). |
| | Proportion of subjects achieving ClinRO for Eyelash Hair |
| | Loss scores of 0 or 1 (full coverage or minimal gaps) with a |
| | ≥2-point improvement from baseline at each visit (among those |
| | with baseline scores ≥2 [significant gaps to no notable hair]). |
| | Change from baseline in ALODEX score at each visit. |
| PD Objective | PD Endpoint |
| To characterize the PD of | Change from baseline in blood biomarkers. |
| Daxdilimab. | |

Abbreviations: AA, Alopecia Areata; ADA, anti-drug antibody; AE, adverse event; AESI, adverse event of special interest; ALODEX, Alopecia Density and Extent; ClinRO, Clinician Reported Outcome; PD, pharmacodynamic; pDC, plasmacytoid dendritic cell; PK, pharmacokinetic; SAE, serious adverse event; SALT, Severity of Alopecia Tool.

### 1) Severity of Alopecia Tool (SALT)

To participate, a subject must have moderate-to-severe AA as assessed by a SALT score of ≥50 and ≤95 at screening and baseline (Day 1). The SALT score is a tool for determining the degree of hair loss based on the percentage of scalp surface area involved on the top, back, and each side of the scalp for AA. The investigator will determine the percent scalp hair loss in each quadrant, multiply this by the total scalp area delineated by that quadrant, and sum the resultant numbers for each quadrant to give the total percent scalp hair loss with a maximum score of 100. The SALT assessment tool is provided in FIG. 2.

### Alopecia Density and Extent (ALODEX)

The ALODEX score combines both extent and hair density into an overall percentage of scalp hair loss. The density scale of 0 to 10 is related to percentage of terminal hair loss, where 100% hair loss is equal to 10, 90% is equal to 9, 80% is equal to 8, 70% is equal to 7, 60% is equal to 6, 50% is equal to 5, 40% is equal to 4, 30% is equal to 3, 20% is equal to 2, 10% is equal to 1 and no hair loss is equal to 0. The density assignments in each of the 1% scalp areas in each quadrant are added together and divided by the maximum grade of hair loss (10) to give the percent hair loss for that quadrant. The score in each quadrant is then added together to give the ALODEX score. The ALODEX assessment is provided in FIG. 3.

### Clinician-Reported Outcomes (ClinROs) for Eyebrow Hair Loss and Eyelash Hair Loss

The ClinRO for eyebrow hair Loss™ and the ClinRO for eyelash hair Loss™ are comprised of single item, 4-point Likert-type response scales to assess incremental severity of each ClinRO measure. The ClinROs are provided in FIG. 4.

### Medical Photography

Medical photographs of the full scalp for all subjects, and of the eyebrows and eyelashes for subjects who have hair loss in these areas at baseline, are performed at the visits specified in the schedule of Table 9. Care are taken to use the same camera, the same magnification, and the same settings for each photograph at each visit to obtain comparable pictures.

### Pharmacokinetics

Blood samples are collected for analysis of Daxdilimab concentrations for all subjects who received ≥1 dose of Daxdilimab 2 hours (±5 minutes) after dosing on Day 1 and pre-dose at all other visits specified in Table 9.

### Pharmacodynamics

Blood samples are collected for PD analysis of Daxdilimab at the visits and timepoints specified in Table 9. The PD analysis of Daxdilimab will include but may not be limited to the assessment of whole blood transcriptomics (ex. IFN gene signature), pDC flow cytometry, PBMCs (ex. levels of other circulating cells), serum and plasma biomarkers, and blood MxA.

### Immunogenicity

Blood samples are collected for analysis of serum Daxdilimab ADA.

### Vital Signs

The following vital signs are recorded at the visits specified in Table 9 with the subject in a seated or supine position, after having sat calmly for at least 5 minutes: systolic and diastolic blood pressure (mmHg), pulse (bpm), body temperature (° C.), and respiratory rate (breaths/minute). Weight (kg) and height (cm) are collected to calculate the BMI and are recorded at the visits specified in Table 9. The height will only be recorded once at the screening visit and the same value are used for BMI calculation at other visits.

### Physical Examination

The following sites/systems will at least be included in the focused physical examination, which are performed at the visits specified in Table 9: (1) General appearance; (2) Dermatological (except AA); (3) Head, eyes, ears, nose, throat (HEENT); (4) Respiratory; (5) Cardiovascular; (6) Abdominal; (7) Neurological; (8) Musculoskeletal; and (9) Lymphatic.

### Clinical Laboratory Tests

Laboratory tests are performed at the visits specified in Table 9. The tests will include urinalysis, hematology with differential, a standard chemistry panel (chemistry includes liver function tests), and serum pregnancy test (screening) for women of childbearing potential (WOCBP). At the visit specified in Table 9, a urine pregnancy test are performed for WOCBP (conducted at the investigator site). The specific tests in these panels are listed in Table 11.

TABLE 11
Clinical Laboratory Testing

| Laboratory Testing | Tests Included |
|---|---|
| Hematology | HCT, Hg, MCH, MCHC, MCV, MPV, PLT, RBC, WBC, and |
| | differentials (neutrophils, lymphocytes, monocytes, eosinophils, and |
| | basophils relative and absolute) |
| Biochemistry | Albumin, alkaline phosphatase, ALT, AST, bicarbonate, calcium, chloride, |
| | creatinine (enzymatic), GGT, glomerular filtration rate (MDRD), glucose |
| | random, LDH, magnesium, potassium, sodium, total bilirubin (including |
| | direct and indirect), triglycerides, urea (BUN), uric acid |
| Urinalysis | Dipstick and microscopic analysis (as required) |
| | Spot UPCr |
| Urine pregnancy test | For female subjects of childbearing potential (at each visit, except screening) |
| Laboratory tests | FSH levels for female subjects who have had a cessation of menses for at |
| required at screening | least 12 months prior to the screening visit without an alternative medical |
| only | cause |
| | Serum IgG (screening). β-hCG for women of |
| | childbearing potential (screening) |
| | Tuberculosis test (PPD or QuantiFERON-TB Gold) |
| | Serology (HBV [HBsAg, HBcAb or anti-HBc], HCV, HIV) |

Abbreviations: ALT, alanine aminotransferase; anti-HBc, antibody to hepatitis B core antigen; AST, aspartate aminotransferase; β-hCG, β-human chorionic gonadotropin; BUN, blood urea nitrogen; FSH, follicle-stimulating hormone; GGT, gamma-glutamyl-transferase; HBcAb, hepatitis B core antibody; HBsAg, hepatitis B surface antigens; HBV, hepatitis B virus; HBcAb, hepatitis B core antibody; HCT, hematocrit; HCV, hepatitis C virus; Hgb, hemoglobin; HIV, human immunodeficiency virus; LDH, lactate dehydrogenase; MCH, mean corpuscular hemoglobin; MCHC, mean corpuscular hemoglobin concentration; MCV, mean corpuscular volume; MDRD, Modification of Diet in Renal Disease; MPV, mean platelet volume; PLT, platelets; PPD, purified protein derivative; RBC, red blood cell (count); UPCr, urine protein to creatinine ratio; WBC, white blood cell (count).

### Electrocardiogram

Twelve-lead ECGs are performed as a safety assessment at the visits specified in Table 4. Clinically significant findings in the ECG should exclude a subject from trial participation. Any clinically significant value are reported as an AE.

### Local Injection Tolerability Assessment

Assessor local injection tolerability assessments are performed approximately 30 minutes post-dose (can be performed any time at Week 36 as no Daxdilimab is administered), at the visits specified in Table 9. The investigator, or designee, will evaluate the injection sites at these visits, and will document the presence or absence of local intolerance/injection site reactions and will open an AE in case of local injection site intolerance. Additional follow-up after Week 36 can be performed for any ongoing injection site reactions.

### Adverse Events

An adverse event (AE) is any untoward medical occurrence in a subject administered a pharmaceutical product and that does not necessarily have a causal relationship with this treatment. An AE can therefore be any unfavorable and unintended sign (including an abnormal laboratory finding), symptom, or disease temporally associated with the use of Daxdilimab, whether or not considered related to the Daxdilimab. A Treatment-Emergent Adverse Event (TEAE) is any condition that was not present prior to treatment with the DAXDILIMAB but appeared following treatment, was present at treatment initiation but worsened during treatment, or was present at treatment initiation but resolved and then reappeared while the individual was on treatment (regardless of the intensity of the AE when the treatment was initiated). A serious adverse event (SAE) or reaction is any untoward medical occurrence that, at any dose has any of the following consequences: results in death, is life-threatening, requires in-patient hospitalization or prolongation of existing hospitalization, results in persistent or significant disability/incapacity, and/or rs a congenital anomaly/birth defect. Guidelines outlined in Common Terminology Criteria for Adverse Events (CTCAE) are utilized.

### Discontinuation

An individual subject will not receive any further Daxdilimab if any of the following occur in the subject:
Receipt of any medications that have been demonstrated to be effective for treatment of AA
A CTCAE Grade 3 or higher allergic reaction to Daxdilimab
A CTCAE Grade 3 or higher infection considered related to Daxdilimab
Other AE that contraindicates further dosing in the opinion of the investigator and/or the Sponsor, or Medical Monitor.
Withdrawal of consent from further treatment with Daxdilimab
Subject is determined to have met one or more of the exclusion criteria or failed to meet all the inclusion criteria for study participation and there is a potential safety risk associated with continuation identified upon consultation with the Medical Monitor.
Pregnancy or a decision to become pregnant.
Any of the following liver function abnormalities:
   ALT or AST≥8×ULN.
   ALT or AST≥5×ULN for more than 2 weeks.
   ALT or AST≥3×ULN and total bilirubin≥2×ULN.
   ALT or AST≥3×ULN with the appearance of fatigue, nausea, vomiting, right upper quadrant pain or tenderness, fever, rash, and/or eosinophilia (≥5%).

### Example 4: A Phase 2 Randomized, Double-Blind, Placebo-Controlled Efficacy and Safety Study of Daxdilimab for Treating Active Proliferative Lupus Nephritis

A phase 2, multicenter, international, double-blind, randomized, placebo-controlled trial are carried out to assess the efficacy and safety of Daxdilimab in subjects with lupus nephritis (LN). The target population is adults aged ≥18 to ≤80 years who have documented active and proliferative lupus nephritis (LN). The maximum study duration per subject is approximately 116 weeks, including approximately 4 weeks (Days −28 to −1) of screening period, 104 weeks of treatment period, 8 weeks (12 weeks after the last study intervention administration at week 100) of safety follow-up (SFU) period.

### Objectives and Endpoints

The primary objective is to evaluate the efficacy of daxdilimab in combination with SOC compared to placebo in combination with SOC in subjects with active, proliferative LN. The primary objective are measured by according to the following criteria: proportion of subjects achieving CRR at Week 48 and sustained through Week 52. CRR is defined as meeting all of the following: (i) eGFR≥60 mL/min/1.73 m² or no worse than 15% below baseline; (ii) 24-hour UPCR≤0.5 mg/mg; and (iii) no discontinuation of study intervention or use of restricted medication beyond the protocol allowed threshold before assessment.

The secondary objective is to assess ORR (defined as CRR plus PRR) with daxdilimab versus placebo in subjects with active, proliferative LN. The secondary object are measured by according to the following criteria: proportion of subjects achieving ORR at Week 48 and sustained through Week 52. PRR is defined as meeting all of the following: (i) eGFR≥60 mL/min/1.73 m2 or no worse than 15% below baseline; (ii) Improvement in 24-hour UPC for subjects with a baseline UPCR≤3.0 mg/mg: <1.0 mg/mg and subjects with a baseline UPCR ≥3.0 mg/mg: ≥50% improvement from baseline and ≤3.0 mg/mg; and (iii) no discontinuation of study intervention or use of restricted medication beyond the protocol allowed threshold before assessment. Other secondary objectives of the study are as follows:
To assess the change from baseline in eGFR with daxdilimab versus placebo in subjects with active, proliferative LN.
   Change from baseline in eGFR at Week 52.
To evaluate the ability to improve dose requirements of oral corticosteroids (OCS) with daxdilimab versus placebo in subjects with active, proliferative LN.
   Proportion of subjects able to taper OCS to ≤2.5 mg/day prednisone-equivalent by Week 24 and maintain this dose through Week 52.
   Sustained reduction of OCS dose:
      Prednisone-equivalent dose≤2.5 mg/day by Week 24 and not exceeding this dose through Week 52, and
      No discontinuation of study intervention or use of restricted medication beyond the protocol allowed threshold before assessment.
To characterize the pharmacokinetics (PK) and immunogenicity of daxdilimab in subjects with active, proliferative LN
   Serum concentration of daxdilimab.
   Rate of anti-drug antibodies (ADA) directed against daxdilimab and ADA titer for the duration of the study.
To evaluate the safety and tolerability of daxdilimab in combination with SOC in subjects with active, proliferative LN.
   Incidence of treatment-emergent adverse events (TEAEs).
   Incidence of treatment-emergent serious adverse events (TESAEs).
   Incidence of treatment-emergent AEs of special interest (TEAESIs): hypersensitivity reaction, including anaphylaxis, severe (Grade 3 or higher) viral infection/reactivation, herpes zoster, opportunistic infection, and malignancy (except non-melanoma skin cancer).
The exploratory objectives and endpoints of the study are as follows:
   i. to evaluate the effect of daxdilimab versus placebo on measures of LN disease activity in subjects with active, proliferative LN. The exploratory endpoints are as follows:
      Proportion of subjects meeting alternative CRR (aCRR) at Weeks 52. aCRR is defined as meeting all of the following:
         eGFR≥60 mL/min/1.73 m² or no worse than 15% below baseline
         24-hour UPCR<0.5 mg/mg^{a}
         Inactive urine sediment (defined as <10 red blood cells [RBC]/high power field [hpf])
         No discontinuation of study intervention or use of restricted medication beyond the protocol allowed threshold before assessment.
      Proportion of subjects achieving renal response (CRR, ORR, aCRR) over time.
      Change from baseline in UPCR over time.
      Change from baseline in eGFR over time.
      Proportion of subjects with eGFR≥60 mL/min/1.73 m2 or no worse than 15% below baseline over time.
      Time to achieve renal response (CRR, ORR, aCRR) sustained to Weeks 52.
      Time to achieve renal response (CRR, ORR, aCRR) sustained to Weeks 52 and taper OCS to ≤2.5 mg/day prednisone-equivalent by Week 24 and maintained through Weeks 52.
   ii. To assess the time to first renal flare in subjects with active, proliferative LN.
      Time from achieving PRR or CRR maintained for at least one subsequent visit to the first renal flare in year 1. Renal flare is defined as an increase in spot UPCR and/or decline in renal function attributable to active LN in subjects who achieved PRR or CRR and then maintained it for at least one subsequent visit. Flares are characterized as either proteinuric or nephritic.
         a) Proteinuric flare:
      (1) ≤20% decrease in eGFR.
      (2) In subjects achieving CRR: Spot UPCR
      (3) 1.0 mg/mg on two urine samples obtained at least 2 weeks apart.
      (4) In subjects achieving PRR: ≥50% increase in spot UPCR from the average of the last two measurements when PRR criteria were met and spot UPCR ≥2.0 mg/mg on two urine samples obtained at least 2 weeks apart.
      b) Nephritic flare: ≥20% decrease in eGFR compared to the average of the previous two visits, not explained by change in comorbidities or concomitant medications. A decrease in eGFR must be confirmed on at least two samples at least 5 days apart after non-systemic lupus erythematosus (SLE) causes have been corrected or excluded. One measurement is acceptable if it leads to an increase in OCS or immunosuppressive therapy.
   iii. To evaluate the effect of daxdilimab versus placebo on SLE disease activity and damage in subjects with active, proliferative LN.
      Change over time in Systemic Lupus Erythematosus Disease Activity Index 2000 (SLEDAI-2K) scores.
      Change over time in non-renal components of SLEDAI-2K scores.
      Change over time in the Systemic Lupus International Collaborating Clinics/American College of Rheumatology (SLICC/ACR) Damage Index (SDI).
      Change over time in the Glucocorticoid Toxicity Index (GTI) score.
   iv. To assess the change from baseline in patient reported outcome scores with daxdilimab versus placebo in subjects with active, proliferative LN.
      Change over time in the Physician's Global Assessment (PGA) scores.
      Change over time in the Patient Global Assessment (PtGA) scores.
      Change over time in the Patient Global Impression of Change (PGI-C) scores.
   v. To characterize the pharmacodynamics (PD) of daxdilimab in subjects with active, proliferative LN.
      Change from baseline in PD biomarkers, including blood pDCs, IFN-alpha, myoxvirus protein A (MxA), and IFN gene signature levels.
      Association of proteinuria with serum concentration of daxdilimab.
   vi. To explore potential associations of genetic variations, gene expression, and profiles of cells and proteins in circulation and urine with daxdilimab response.
      Change over time in blood biomarkers.
      Change over time in urinary biomarkers.
      Change over time in whole blood gene expression.
   vii. To assess changes on renal biopsy tissue in subjects with active, proliferative LN.
      Change from baseline in the International Society of Nephrology (ISN)/Renal Pathology Society (RPS) [ISN/RPS] classification and National Institutes of Health (NIH) activity and chronicity indices.
      Change from baseline in pDCs and other tissue biomarkers.

### Study Design

An overview of the trial design is presented in the schematic provided in FIG. 7.

Approximately 210 subjects are randomized in a ratio of 1:1:1 (70 subjects per group) to receive either daxdilimab SC or placebo SC in addition to SOC background therapy as described below in Table 12. Randomization is stratified by:
Pre-randomization urine protein to creatinine ratio (UPCR)<3.0 mg/mg vs. ≥3.0 mg/mg
Screening eGFR<60 mL/min/1.73 m2 vs. ≥60 mL/min/1.73 m2

TABLE 12
First Year Treatment Assignment

| Group | Treatment |
|---|---|
| 1 | Day 1 through Week 52: Daxdilimab 100 mg SC at |
| | baseline, Week 2, and Week 4, followed by |
| | 100 mg SC Q4W through Week 52. |
| 2 | Day 1 through Week 52: Daxdilimab 300 mg SC at |
| | baseline, Week 2, and Week 4, followed by |
| | 300 mg SC Q4W through Week 52. |
| 3 | Day 1 through Week 52: Placebo SC at baseline, |
| | Week 2, and Week 4, followed by placebo SC |
| | Q4W through Week 52. |

At Week 64, subjects from all three Treatment Groups (above) are assigned to a quarterly dosing maintenance regimen based upon the renal response observed at Weeks 48 and 52, as described below in Table 13.

TABLE 13
Second Year Treatment Assignment

| Group | Treatment |
|---|---|
| | Week 64 through Week 104: |
| 1 | PRR (or CRR) at both Weeks 48 and 52: daxdilimab 100 mg SC Q12W through |
| | Week 104 (last dose administered at Week 100) OR |
| | No PRR (or CRR) at either Week 48 or Week 52: daxdilimab 300 mg SC Q12W |
| | through Week 104 (last dose administered at Week 100) |
| | Week 64 through Week 104: |
| 2 | PRR (or CRR) at both Weeks 48 and 52: daxdilimab 300 mg SC Q12W through |
| | Week 104 (last dose administered at Week 100) OR |
| | No PRR (or CRR) at either Week 48 or Week 52: daxdilimab 300 mg SC Q12W |
| | through Week 104 (last dose administered at Week 100) |
| | Week 64 through Week 104: |
| 3 | PRR (or CRR) at both Weeks 48 and 52: placebo SC Q12W through Week |
| | 104 (last dose administered at Week 100) OR |
| | No PRR (or CRR) at either Week 48 or Week 52: daxdilimab 300 mg SC Q12W |
| | through Week 104 (last dose administered at Week 100) |

Subjects may enter the study taking daily oral corticosteroids (OCS) at a maximum dose of 0.5 mg/kg/day of prednisone-equivalent, not to exceed 40 mg/day, and must be on a stable dose for at least 10 days prior to randomization. In addition, subjects will receive IV methylprednisolone pulse 500 mg on the day of Randomization, prior to receiving the study intervention, unless they received a methylprednisolone pulse of ≥500 mg within 10 days prior to randomization followed by a reducing taper of OCS for subjects already taking OCS prior to entry. For subjects who are not already taking prescribed mycophenolate mofetil (MMF) prior to Randomization, the dosing of MMF will start at 500 mg twice a day (BID) for a total daily dose of 1 gm/day for the first week, increasing to 1 gm BID for a total daily dose of 2 gm/day for the second and subsequent weeks (i.e., beginning on Day 8). An equivalent dose of mycophenolic acid (MPA) may be used as an alternative to MMF.

The trial will comprise a Screening Period of approximately 4 weeks (Days −28 to −1), a Treatment Period with Randomization on Day 1 followed by treatment through Week 104 (last dose administered at Week 100) totaling 19 administrations for all Treatment Groups with assignment of a quarterly dosing maintenance regimen at Week 64 based upon the renal response observed at Weeks 48 and 52, and a SFU Period of 8 weeks (through Week 112). Under exceptional circumstances such as delayed laboratory results, drug washout, or the impact of Coronavirus Disease 2019 (COVID-19), the Screening Period may be increased by 2 weeks, upon approval by the Medical Monitor.

Rescue therapy for worsening LN may be administered at the discretion of investigators as clinically indicated. Rescue therapy includes the initiation of any new treatment for lupus or LN or an increase from baseline in the SOC background therapy (MMF/MPA or corticosteroids).

The primary assessment of the efficacy endpoints is performed at Weeks 48 and 52. The final evaluation of the double-blind Treatment Period will occur at Week 104. The SFU Period aregin after completion of the Week 104 EOT Visit and will last for 8 weeks. All subjects will continue to receive SOC immunosuppressive background therapy with MMF/MPA and OCS, unless the subject has successfully discontinued OCS. Subjects will continue to follow all trial requirements and will return to the clinical site for the Week 112 End-of-Study Visit for final safety assessments.

### Inclusion Criteria

All subjects must meet/provide all of the following criteria to be eligible for trial participation:
1. Written informed consent and any locally required authorization prior to performing any protocol-related procedures and screening evaluations.
2. Willing and able to comply with the prescribed treatment protocol and evaluations for the duration of the trial.
3. Adult men or women ≥18 and ≤80 years of age.
4. Fulfill the 2019 European League Against Rheumatism/American College of Rheumatology Classification Criteria for systemic lupus erythematosus (SLE).
5. Have at least one of the following at Screening per central lab:
   Antinuclear antibodies (ANA)≥1:80.
   Anti-double stranded deoxyribonucleic acid (anti-dsDNA) antibodies elevated to above normal range as established by the central laboratory (i.e., positive results).
   Anti-Smith antibodies elevated to above normal (i.e., positive results).
6. Diagnosis of proliferative LN based on a renal biopsy obtained within 6 months prior to signing the informed consent form (ICF) or during the Screening Period:
   Class III (±class V) or class IV (±class V) LN. The submission of the Screening biopsy sample for adjudication is required to participate in the study.
7. Urine protein to creatinine ratio ≥1.5 mg/mg (113.17 mg/mmol), obtained via a 24-hour urine collection at both:
   The start of Screening, and
   Within 14 days of expected date of Randomization.
8. Estimated glomerular filtration rate ≥35 mL/min/1.73 m2.
9. Negative serum β human chorionic gonadotropin (β-hCG) test for female subjects.
10. Women of childbearing potential must have negative serum and urine pregnancy tests, and men must agree to use appropriate contraception so as to not impregnant a female partner and refrain from donating sperm during the trial.

### Exclusion Criteria

Subjects are ineligible for trial participation if they meet any of the following criteria at the Screening and/or Day 1 Visits, as applicable:
1. Weight ≥160 kg (352 pounds) at Screening.
2. History of allergy, hypersensitivity reaction, or anaphylaxis to any component of the IP or to a previous monoclonal antibody (mAb) or human immunoglobulin (Ig) therapy.
3. Known intolerance to ≤1.0 gm/day of MMF or equivalent dose of MPA.
4. Participation in another clinical study with an investigational drug within 4 weeks prior to Day 1 or within 5 published half-lives, whichever is longer.
5. Breastfeeding or pregnant women or women who intend to become pregnant anytime from signing the ICF through 6 months after receiving the last dose of IP.
6. History of drug or alcohol abuse
7. Major surgery within 8 weeks prior to Screening or elective surgery planned from Screening through the end of the trial.
8. Spontaneous or induced abortion, still or live birth, or pregnancy ≤4 weeks prior to Screening through Randomization.
9. A diagnosis of pure Class V membranous LN based on a renal biopsy obtained within 6 months prior to signing ICF or during the Screening Period.
10. History of dialysis within 12 months prior to signing the ICF or expected need for renal replacement therapy (dialysis or renal transplant) within a 12-month period after enrollment.
11. History of, or current renal diseases (other than LN) that in the opinion of the Investigator could interfere with the LN assessment and confound the disease activity assessment (e.g., diabetic nephropathy).
12. Known history of a primary immunodeficiency or an underlying condition such as known human immunodeficiency virus (HIV) infection, a positive result for HIV infection per central laboratory, splenectomy, or any underlying condition that in the opinion of the Investigator significantly predisposes the subject to infection.
13. During Screening, any of the following per central laboratory:
   Aspartate aminotransferase ≥2.5×upper limit of normal (ULN)
   Alanine aminotransferase ≥2.5×ULN
   Total bilirubin≥1.5×ULN (unless due to Gilbert's syndrome)
   Serum IgG <600 mg/dL (or <6 g/L)
   Neutrophil count <1000/μL (or <1.0×10⁹/L) or <500/μL (<0.5×10⁹/L) if due to active SLE
   Platelet count <50,000/μL (or <50×10⁹/L) or <25,000/μL (<25×10⁹/L) if due to active SLE
   Hemoglobin <8 g/dL (or <80 g/L) or <7 g/dL (<70 g/L) if due to active SLE
   Glycosylated hemoglobin ≥8% (or ≥0.08)
   Total lymphocyte count <200 cells/mm³
14. Confirmed positive test for hepatitis B serology defined as:
   Hepatitis B surface antigen, or
   Hepatitis B core antibody (HBcAb) and hepatitis B virus (HBV) DNA detected above the lower limit of quantitation (LLOQ) by reflex testing
15. Positive test for hepatitis C virus antibody.
16. Active tuberculosis (TB), or a positive IFN-gamma release assay (IGRA) test at Screening.
17. Any severe herpes virus family infection.
18. Any herpes zoster, CMV, or Epstein-Barr virus infection.
19. Any of the following within 30 days prior to signing the ICF and through Randomization:
   Clinically significant active infection requiring antibiotics or antiviral medication.
   Any infection requiring hospitalization or treatment with intravenous anti-infectives.
   A subject with a documented positive severe acute respiratory syndrome coronavirus 2 (SARS-COV-2) or carrying epidemiologic risk of COVID-19
20. Opportunistic infection requiring hospitalization or parenteral antimicrobial treatment within 2 years prior to Randomization.
21. Any acute illness or evidence of clinically significant active infection on Day 1.
22. Clinically significant cardiac disease including unstable angina, myocardial infarction, congestive heart failure within 6 months prior to Randomization, including inadequately controlled arrythmia and the presence of abnormality on electrocardiogram (ECG).
23. History of cancer within the past 5 years, except as follows:
   In situ carcinoma of the cervix treated with apparent success with curative therapy ≥12 months prior to Screening, or
   Cutaneous basal cell or squamous cell carcinoma treated with curative therapy.
24. Receipt of a live vaccine within 4 weeks prior to Day 1.
25. Active severe or unstable neuropsychiatric SLE including, but not limited to: aseptic meningitis, cerebral vasculitis, myelopathy, demyelination syndromes (ascending, transverse, acute inflammatory demyelinating polyradiculopathy), acute confusional state, impaired level of consciousness, psychosis, acute stroke or stroke syndrome, cranial neuropathy, status epilepticus, cerebellar ataxia, and mononeuritis multiplex:
   a) That might cause the subject to be unable to fully understand the ICF; or
   b) Protocol specified SOC is insufficient to control neurologic features of SLE and utilization of a more aggressive therapeutic approach, such as adding IV cyclophosphamide and/or high dose IV pulse corticosteroid therapy or other treatments not permitted in the protocol, is indicated or anticipated.
26. Documented history of systemic sclerosis or diagnosis of SLE with overlapping systemic sclerosis.
27. History of, or current diagnosis of, catastrophic anti-phospholipid syndrome (APS) or APS-related thromboembolic event or pregnancy loss within 1 year prior to signing the ICF. Subjects with APS adequately controlled by anticoagulants or aspirin for at least 12 weeks may be recruited to the study.
28. History of any non-SLE disease that has required treatment with oral or parenteral corticosteroids for more than a total of 2 weeks within the last 24 weeks prior to signing the ICF.
29. Prior and Concomitant Therapy Criteria: receipt of any of the following treatments within the following timeframes:
   6 weeks prior to Randomization:
      Opioid use above 40 mg/day morphine-equivalent, unstable dosing, or initiation of regular dosing
      IV corticosteroids ≥3.0 gm (cumulative dose)
   8 weeks prior to Randomization:
      Immunoglobulins (except anti-SARS-COV-2 therapeutic antibodies)
      Calcineurin inhibitors (e.g., cyclosporin, voclosporin, tacrolimus), mechanistic target of rapamycin inhibitors, retinoids, thalidomide, lenalidomide, or Janus kinase inhibitors
      Transfusion with blood, packed red blood cells, platelets or treatment with plasmapheresis, plasma exchange, or Therakos® photopheresis
   12 weeks (or 5 half-lives, whichever is longer) prior to Randomization:
      IV cyclophosphamide ≥2 pulses of high dose (≥0.5 gm/m2) or ≥4 doses of low dose (500 mg every 2 weeks)
      Alkylating agents other than cyclophosphamide (e.g., chlorambucil)
      Cytokine or cytokine receptor antagonists, including but not limited to interleukin (IL)-1, IL-6, IL 17, IL-12/23, IL-23, IFN, integrin, or TNFα antagonists (except for IFN-α kinoid, for which receipt at any time is exclusionary)
      Belimumab, abatacept, or eculizumab
      Other biologics used for immunosuppression or immunomodulation (eg, IFN therapy, IL-2)
      Investigational drugs
      IPP-201101 (Lupuzor™)
   24 weeks prior to Randomization:
      B cell-depleting therapies (eg, rituximab, ocrelizumab, ofatumumab, inebilizumab, telitacicept) other than atacicept or obinutuzumab
      Receipt of systemic glucocorticoids (ie, PO, rectal, IV or IM) for more than a total of 2 weeks for any concurrent illness, including asthma, inflammatory bowel disease, or drug-induced SLE
   40 weeks prior to Randomization:
      Atacicept
   1 year prior to Randomization:
      Bacille-Calmette-Guerin (BCG) vaccination
   1.5 years prior to Randomization:
      Obinutuzumab
   The following medications must be discontinued prior to the day of Randomization:
      Methotrexate, Azathioprine, Leflunomide, Mizoribine, Proton pump inhibitors (eg, omeprazole, esomeprazole, lansoprazole, pantoprazole, etc.), and Cholestyramine
      Corticosteroids with a long biologic half-life (i.e., dexamethasone, betamethasone)

### Concomitant Therapies

Permitted medications for standard-of-care (SOC) treatment for LN are described below. The SOC for LN will consist of the combination of MMF/MPA and corticosteroids.
1) Mycophenolate Mofetil (MMF)/Mycophenolic Acid (MPA): The target dose of MMF is 2 gm/day by mouth throughout the study, where the dose is titrated to the target dose between Randomization and Week 8. For subjects who are not already taking prescribed MMF prior to Randomization, the dosing of MMF will start at 500 mg BID for a total daily dose of 1 gm/day for the first week, increasing to 1 gm BID for a total daily dose of 2 gm/day for the second and subsequent weeks (i.e., beginning on Day 8). An equivalent dose of MPA may be used as an alternative to MMF. All subjects will take MMF/MPA BID (i.e., morning and evening), before meals (i.e., on an empty stomach), with a glass of water. A maximum dose of 3.0 gm/day is allowed up to Week 24 for subjects with suboptimal response between Weeks 8 and 24 with a reduction to ≤2.0 gm/day by Week 32. The dose of MMF must be stable from Week 40 to Week 52. After Week 52, MMF dose should be ≤2 gm/day. If MMF dose >2 gm/day at Week 52, taper to ≤2 gm/day is recommended by Week 60. The dose of MMF must be stable from Week 92 to Week 104. The use of IV forms of mycophenolate is prohibited.
   a) Initial adjustment of MMF/MPA dose to achieve or maintain the target dose (Randomization through Week 8)
      i) Subjects who are receiving MMF/MPA at a dose of 2 gm/day (or equivalent) at Week 0 (Day 1) will continue this dose without interruption.
      ii) Subjects who are not taking MMF/MPA at Week 0 (Day 1) or subjects who are taking less than 2 gm/day (or equivalent), the dose is titrated up with the goal of achieving a dose of 2 gm/day (or the therapeutic target dose if local treatment standards dictate a lower dose) by Week 1 (Day 8) (but not later than Week 8 (Day 57), if dose escalation is limited by intolerability).
      iii) Subjects who at Week 0 (Day 1) are taking MMF doses >2 gm/day will have their dose reduced to 2 gm/day by Week 8 (Day 57) unless they meet criteria for suboptimal response (see below for suboptimal response).
   b) MMF/MPA dose between Week 8 and 24
      i) The dose of MMF/MPA is kept stable from Week 8 through Week 24 unless dose escalation to a maximum of 3 gm/day is necessary for suboptimal response (defined below) or a dose reduction is necessary to manage toxicity or intolerability.
      ii) Criteria for suboptimal response: A suboptimal response is defined by UPCR values shown below at two independent measurements taken at least 2 weeks apart:
         (1) Spot UPCR>3 mg/mg and
         (2) Spot UPCR<15% decrease compared to baseline spot UPCR
      iii) If a subject meets the criteria at or after Week 8, the dose may be escalated or kept above 2 gm/day. After initial confirmation of suboptimal response, MMF/MPA can be increased up to 3 gm/day (or equivalent) until Week 16 without re-testing for the suboptimal response criteria. The dose of MMF/MPA must be kept stable from Week 16 unless dose reduction is necessary for intolerability or MMF/MPA-related AEs.
   c) MMF/MPA dose between Week 24 and Week 52:
      i) For subjects who are taking ≥2 gm/day of MMF (or MPA equivalent) at Week 24, the dose of MMF/MPA must be decreased to a maximum dose of 2 gm/day (or equivalent) by Week 32. The dose of MMF/MPA must be stable from Week 40 through Week 52 (unless a dose reduction is necessary to manage toxicity or intolerability).
   d) MMF/MPA dose from Week 52 to Week 104:
      i) The target dose of MMF/MPA is about ≤2 gm/day. If the MMF/MPA dose is ≥2 gm/day at Week 52, it should be tapered to ≤2 gm/day by Week 60. MMF/MPA dose must not be changed from Week 92 to Week 104.
2) Corticosteroids
   a) Initial corticosteroid treatment to control LN and SLE: Subjects may enter the study taking daily OCS at a maximum dose of 0.5 mg/kg/day of prednisone-equivalent, not to exceed 40 mg/day, and must be on a stable dose for at least 10 days prior to Randomization. In addition, subjects will receive IV methylprednisolone pulse 500 mg on the day of Randomization, prior to receiving the study intervention, unless they received a methylprednisolone pulse of ≥500 mg within 10 days prior to Randomization.
   b) Steroid tapering during the study: Oral corticosteroid dose tapering is required during the study with the goal of fractionally reduce OCS to a prednisone-equivalent dose of ≤7.5 mg/day by Week 12 and prednisone-equivalent dose of ≤2.5 mg/day by Week 24. Steroid tapering will not be permitted from Week 40 to Week 52. A recommended tapering schedule is provided below in Table 14.

TABLE 14
Exemplary Oral Prednisone Tapering Schedule

| Starting Prednisone | 10 | 20 | 30 | 40 |
|---|---|---|---|---|
| Dose (mg/day) | mg/day | mg/day | mg/day | mg/day |
| Week 2 (Day 15) | 10 | 15 | 25 | 35 |
| Week 4 (Day 29) | 10 | 15 | 25 | 30 |
| Week 6 (Day 43) | 7.5 | 10 | 20 | 25 |
| Week 8 (Day 57) | 7.5 | 10 | 15 | 20 |
| Week 10 (Day 71) | 5 | 7.5 | 10 | 15 |
| Week 12 (Day 85) | 5 | 7.5 | 7.5 | 7.5 |
| Week 16 (Day 113) | 2.5 | 5 | 5 | 5 |
| Week 20 (Day 141) | 2.5 | 2.5 | 2.5 | 2.5 |
| Week 24 (Day 169) | 2.5 | 2.5 | 2.5 | 2.5 |

c) Steroid “burst and taper”:
   i) A steroid burst is defined as one of the following:
      (1) OCS increase up to a maximum daily dose of 0.5 mg/kg/day (maximum 40 mg/day) prednisone-equivalent dose for up to a total of 14 days which must be fully administered and tapered to less than or equal to the pre-burst starting dose by the end of the 14th day. Any course of OCS burst must not extend beyond Week 40, regardless of when the course was started, or
      (2) A maximum of 1 instance of intra-articular, tendon sheath or bursal injections (for a total dose of methylprednisolone ≤80 mg or equivalent) can be given. Subjects who receive any intraarticular/tendon sheath/bursal injections should not receive OCS burst (and vice versa).
   ii) Steroid burst is allowed as follows:
      (1) From Randomization to Week 40: One burst and taper of corticosteroids for increased SLE disease activity or for non-SLE activity is allowed.
      (2) Increase in corticosteroids from Week 40 to Week 52: No increase in OCS, or the use of IV or intra-articular, tendon sheath or bursal injections is allowed from Week 40 until the Week 52 assessment.
      (3) One burst and taper is allowed between Week 52 and Week 92.
d) Increase in corticosteroids for the prevention of adrenal insufficiency
   i) For a severe illness, surgery, or symptoms of adrenal insufficiency, if clinically warranted, the following can be used, in addition to the “burst and taper” described above from Randomization to Week 40 without the subject being considered a non-responder:
      (1) Oral or IV hydrocortisone up to 100 mg every 8 hours on the first day followed by half of the previous dose for 2 days before returning to their usual dose OR
      (2) Subjects who are taking ≤7.5 mg/day prednisone or equivalent are allowed to receive up to an additional 7.5 mg/day to a total of 15 mg/day oral prednisone or equivalent for a total of up to 14 days.
   ii) Subjects who receive either of these corticosteroid regimens for the prevention of adrenal insufficiency between Week 40 and Week 52 are considered as a non-responder for the analyses at Week 52.

### Concomitant Medications

Medications other than the SOC immunosuppressive background therapy may be given: (i) Anti-Hypertensive Agents and HMG-COA Reductase Inhibitors (Statins) but chloestyramine must be discontinued prior to the day of Randomization, (ii) Sodium-Glucose Cotransport 2 Inhibitors, (iii) Anti-Malarial Agents, (iv) Non-Steroidal Anti-inflammatory Drugs, (v) Acetaminophen (Paracetamol), (vi) Narcotic Analgesics, and (vii) Topical Therapy.

### Criteria for Discontinuing Study Intervention for Worsening LN at any Time

Blinded study intervention is discontinued in subjects who meet pre-defined criteria for worsening LN or SLE: (i) >30% decrease in eGFR compared to baseline due to LN and eGFR <60 mL/min/1.73 m²; (ii) Increase in renal or extra-renal lupus activity requiring prohibited systemic immunosuppressive treatment (e.g., cyclophosphamide, rituximab, belimumab); (iii) Receipt >1 methylprednisolone pulse after the day of Randomization; (iv) Receipt of any methylprednisolone pulse after Week 8; or (v) if MMF is discontinued, and another immunosuppressant is initiated.

### Criteria for Discontinuing Study Intervention for Worsening LN at Weeks 12-24

Blinded study intervention is discontinued in subjects who meet pre-defined criteria for worsening LN or SLE at Weeks 12-24: (i) eGFR <75% of baseline and <60 mL/min/1.73 m2; (ii) nephrotic range UPCR for subjects with 24-hour UPCR ≤3 mg/mg at baseline showing increases at 24-hour by >50% from baseline to >3.5 mg/mg, and for subjects with 24-hour UPCR >3 mg/mg at baseline showing 3.5 mg/mg and there is <50% improvement from baseline at Week 24, or (iii) inability to adhere to corticosteroids requirements: inability to reduce OCS to ≤15 mg/day prednisone-equivalent at Week 12 or inability to reduce OCS to ≤10 mg/day prednisone-equivalent by Week 24.

### Early Discontinuation of Study

An individual subject will not receive any further study intervention if any of the following occur in the subject:
Receipt of Prohibited Medications, a Common Terminology for Adverse Events, a Grade 3 or higher allergic reaction to the study intervention, a CTCAE Grade 3 or higher infection considered related to the study intervention, or other AE that contraindicates further dosing in the opinion of the Investigator and/or the Sponsor or medical monitor.
Any of the following liver function abnormalities:
   alanine aminotransferase (ALT) or aspartate aminotransferase (AST)≥8× ULN.
   ALT or AST≥5×ULN for more than 2 weeks.
   ALT or AST≥3×ULN and total bilirubin≥2×ULN.
   ALT or AST≥3×ULN with the appearance of fatigue, nausea, vomiting, right upper quadrant pain or tenderness, fever, rash, and/or eosinophilia (≥5%).

### Daxdilimab Composition, Dosage, and Mode of Administration

Daxdilimab is supplied as a 2R vial with nominal 1 mL of 100 mg/mL daxdilimab containing 20 mM L-histidine/L-histidine HCl, 240 mM sucrose, 0.02% (w/v) polysorbate 80, pH 6.0. Randomized subjects will receive either daxdilimab 100 mg or 300 mg administered subcutaneously (SC) as two 1.5 mL injections at baseline, Week 2, and Week 4, followed by daxdilimab SC every 4 weeks (Q4W) through Week 52 in addition to standard-of-care (SOC) therapy. At Week 64, all subjects are assigned to a quarterly dosing maintenance regimen based upon the renal response observed at Weeks 48 and 52. All subjects, regardless of treatment assignment, who are unable to achieve at least a partial renal response (PRR) at either Week 48 or Week 52 are administered daxdilimab 300 mg SC as two 1.5 mL injections at Week 64 and every 12 weeks (Q12W) thereafter through Week 104 (last dose administered at Week 100) in addition to SOC therapy. Subjects who achieve at least a PRR (or Complete Renal Response [CRR]) at both Weeks 48 and 52 and were randomized to daxdilimab treatment with either 100 mg or 300 mg are assigned to a maintenance regimen with the same dose (ie, either 100 mg or 300 mg) at Week 64 and Q12W thereafter through Week 104 (last dose administered at Week 100) in addition to SOC therapy.

As a reference, placebo is procured as commercially available Normal Saline and is administered SC to randomized subjects at baseline, Week 2, and Week 4, followed by placebo SC Q4W through Week 52 in addition to SOC therapy. At Week 64, subjects who were originally assigned to treatment with placebo and achieved at least a PRR (or CRR) at both the Week 48 and Week 52 visits will continue to receive placebo SC as two 1.5 mL injections Q12W through Week 104 (last dose administered at Week 100) in addition to SOC therapy. Randomization is stratified by: (1) pre-randomization urine protein to creatinine ratio (UPCR)<3.0 mg/mg vs. ≥3.0 mg/mg, and (2) Screening estimated glomerular filtration rate (eGFR)<60 mL/min/1.73 m² vs. ≥60 mL/min/1.73 m².

Further details regarding the daxdilimab and placebo administration can be found in Table 15 below.

TABLE 15
Study Interventions Administered

| Intervention Label | Investigational Product | Placebo |
|---|---|---|
| Intervention Name | Daxdilimab | Placebo |
| Intervention Description | Nominal 1 mL of 100 mg/mL | Normal Saline |
| | daxdilimab containing 20 mM | |
| | L-histidine/L-histidine HCl, 240 | |
| | mM sucrose, 0.02% (w/v) | |
| | polysorbate 80, pH 6.0. | |
| Type | Active Drug | Placebo |
| Dose Formulation | 2R vial | Commercially available saline |
| Dosage Level(s) | 100 mg or 300 mg dose as 2 × 1.5 | 2 × 1.5 mL at baseline, Week 2, |
| | mL SC injections at baseline, | Week 4 and followed by Q4W for |
| | Week 2, Week 4 and followed by | 52 weeks followed by Q12W |
| | Q4W for 52 weeks followed by | starting at Week 64 through Week |
| | Q12W starting at Week 64 | 104 (last dose administered at |
| | through Week 104 (last dose | Week 100; total of 19 doses). |
| | administered at Week 100; total | |
| | of 19 doses). | |
| Route of Administration | SC injection | SC injection |
| Administration | Daxdilimab should be | Placebo should be |
| Instructions | administered by clinic staff | administered by clinic staff |
| | trained in best practices for SC | trained in best practices for SC |
| | administration of treatments. | administration of treatments. |

### Duration of Treatment and Follow-Up

Screening is completed within 28 days prior to the Day 1 Visit.

Double-Blind Treatment Period (Day 1 through Week 104): Study treatment (daxdilimab or placebo) is administered SC Q4W through Week 52 with an additional dose at Week 2; at Week 64, all subjects are assigned to a quarterly dosing maintenance regimen based upon the renal response observed at Weeks 48 and 52 and will either receive daxdilimab or placebo Q12W through Week 104 (last dose administered at Week 100). Final evaluation will occur at Week 104.

End-of-Study/Early Termination Visit: Week 112 or earlier if the subject withdraws consent to participate in the trial.

Safety Follow-Up: Subjects should be followed to assess AE/SAEs for at least 12 weeks after the last study intervention administration, approximately 4, 8, and 12 weeks after the last study intervention dose, unless the subject withdraws consent.

### Efficacy Assessment

Efficacy is assessed by renal responses, change in proteinuria, and GFR. Subject's experience is assessed by the Patient Global Assessment (PtGA) and the Patient Global Impression of Change (PGI-C) scales. Efficacy measurements are made at the times indicated in Tables 12 and 13A-C.

Analyses of the Primary Efficacy Endpoint: The proportion of subjects achieving CRR at Week 48 and sustained through Week 52 in the daxdilimab group is compared to that of the placebo group using a logistic regression model with treatment, baseline eGFR value and baseline UPCR value included in the model.

Analyses of the Secondary Efficacy Endpoints: ORR at Week 48 and sustained through Week 52 analyzed using a logistic regression model with treatment, baseline eGFR value and baseline UPCR value included in the model. Response in sustained reduction of OCS dose are analyzed similarly using a logistic regression model with treatment, randomization stratification factor and baseline OCS dose included in the model. Change from baseline in eGFR at Week 52 are analyzed using mixed models for repeated measures (MMRM) with treatment, visit, visit by treatment interaction, randomization stratification factor (UCPR only) and baseline eGFR value included in the model.

The efficacy assessments based on laboratory tests include:
1) Urine protein to creatinine ratio (UPCR): The 24-hour UPCR are determined via a 24-hour urine sample during the Screening period, Weeks 0, 12, 24, 36, 48, 52, 64, 76, 88, 100, 104 and at ET Visits. Starting from Week 0 (Day 1), the 24-hour urine sample must always be collected before the administration of study intervention. The sample collected at Week 0 (Day 1) are the baseline sample which must be provided on Day 1 prior to Randomization and administration of first dose of study intervention. The 24-hour UPCR are used for the primary and secondary endpoints, the PRR and CRR criteria at Weeks 48 and 52, and to determine withdrawal criteria at Weeks 12 and 24. The 24-hour UPCR may also be used for other exploratory endpoints and are detailed in the SAP. Spot UPCR are performed at all visits and are used for the calculation of the SLEDAI-2K and other exploratory endpoints including the CRR and PRR classification for flare assessments.
2) The efficacy assessments based on clinical evaluation include SLEDAI-2K, PGA, OCS reduction, SDI, and GTI.
   a) SLEDAI-2K scoring:
      i) Mild or moderate flare per expert clinician's opinion: change in SLEDAI-2K score of 3 points or more (but not to more than 12), new or worse in discoid, photosensitive, profundus, cutaneous vasculitis, bullous lupus, nasopharyngeal ulcers, pleuritis, pericarditis, arthritis, fever, equal or more than 1.0 increase in PGA score but not to more than 2.5.
      ii) Severe flare: change in SLEDAI-2K score to greater than 12, new or worse CNS-SLE, vasculitis, nephritis, myositis, platelets<60,000, hemolytic anemia: hemoglobin<70 g/L or decrease in hemoglobin>30 g/L, hospitalization for SLE Activity, increase in PGA to >2.5.
   b) A trained and certified investigator will complete the PGA, representing the physician's overall assessment of average disease severity on a Visual Analogue Scale (VAS) with 0 (none) to 3 (severe disease activity over the last 30 days.
   c) The SDI is used to assess irreversible damage in SLE subjects independently of its cause (ie, included damage due to SLE activity, SLE-related scarring, therapy, comorbidities) but occurring after disease onset.
   d) The GTI is used to assess glucocorticoid-related morbidity and glucocorticoid-sparing ability of other therapies.
   e) Subjects are asked to complete the PtGA and the patient lobal impression of change (PGI-C).
   f) Renal Biopsy if needed.

### Pharmacokinetics, Pharmacodynamics, and Immunogenicity

The PK of daxdilimab are assessed by serum PK concentrations. Daxdilimab immunogenicity are assessed by the rate and titer of ADA. Daxdilimab effect on pharmacodynamic (PD) biomarkers are assessed by measuring plasmacytoid dendritic cells (pDCs) in peripheral blood, IFN-alpha protein, myoxvirus protein A (MxA), and IFN gene signature levels. Change from baseline in blood pDC level, rate of positive ADA and ADA titer are evaluated by treatment and by visit. In consenting subjects, DNA are isolated from a blood sample collected at Baseline to test for single nucleotide polymorphisms (SNP) in genes that may be related to the development of lupus or to the mechanism of action of daxdilimab (eg, Fc gamma receptor). Daxdilimab immunogenicity are assessed by the incidence and titer of anti-drug antibodies prior to the daxdilimab administration and at specified time points.

Blood samples are collected in serum separator collection tubes to evaluate PK at the following visits: pre- and 6 hours (±1 hour) post-injection on Day 1, prior to SC administration on Weeks 2, 4, 8, 12, 16, 20, 24, 28, 32, 36, 40, 44, 48, 52, 64, 76, 88, 100, and 104.

### Safety

Safety are assessed and will include monitoring and recording of all AEs, whether or not drug-related, measurement of vital signs, physical examinations, and monitoring of hematology, blood chemistry, and urinalysis by performing clinical laboratory testing (Table 16). AEs are coded using the most recent version of Medical Dictionary for Regulatory Activities (MedDRA). The number and percentage of subjects reporting TEAEs are summarized for each treatment group by MedDRA System Organ Class and preferred terms (PT), by severity, and by relationship to the study intervention. Other safety parameters, including but not limiting to laboratory assessments and vital signs will also be measured as appropriate.

TABLE 16
Laboratory testing

| Laboratory | |
|---|---|
| Testing | Tests Included |
| Hematology | HCT, Hgb, MCH, MCHC, MCV, MPV, PLT, RBC, WBC, and |
| | differentials (neutrophils, lymphocytes, monocytes, eosinophils, and |
| | basophils relative and absolute) |
| Biochemistry | Albumin, alkaline phosphatase, ALT, AST, bicarbonate, calcium, |
| | chloride, creatinine (enzymatic), GGT, glomerular filtration rate |
| | (MDRD), glucose random, LDH, magnesium, potassium, sodium, |
| | total bilirubin (including direct and indirect), triglycerides, urea |
| | (BUN), uric acid |
| Urinalysis | Dipstick and microscopic analysis (as required) Spot UPCR |
| Urine pregnancy | For female subjects of childbearing potential (at each visit, except |
| test | Screening) |

### Example 5: A Phase 2 Randomized, Double-Blind, Placebo-Controlled Efficacy and Safety Study of Daxdilimab for Treating Moderate-to-Severe Primary Discoid Lupus Erythematosus

A Phase 2, multicenter, randomized, double-blind, placebo-controlled, parallel-group trial to investigate the efficacy and safety of daxdilimab subcutaneous injection in reducing disease activity in adult subjects with moderate-to-severe primary discoid lupus erythematosus.

The primary objective of the study is to evaluate the effect of daxdilimab compared with placebo in reducing active disease activity at Week 24 in participants with primary DLE.

The secondary objectives of the study are: (i) to evaluate the effect of daxdilimab compared with placebo in reducing DLE disease activity at Week 24 in subjects with primary DLE; (ii) to evaluate the effect of daxdilimab compared with placebo in disease activity and damage in subjects with primary DLE; (iii) to characterize the PK and immunogenicity of daxdilimab in subjects with primary DLE; and (iv) to evaluate the safety and tolerability of daxdilimab in subjects with primary DLE.

The exploratory objectives of the study include: (i) to evaluate the effect of daxdilimab compared with placebo on other measures of DLE disease activity; (ii) to characterize the PD of daxdilimab; (iii) to explore potential associations of genetic variations, gene expression, and profiles of cells and proteins in circulation and tissue with daxdilimab; and (iv) To evaluate the effect of daxdilimab on the subject's perceived burden and impact of DLE.

### Trial Design

This is a Phase 2, multicenter, double-blind, randomized, placebo-controlled, parallel-group trial with subjects aged ≥18 to ≤75 years who have moderate-to-severely active (i.e., recent flares or chronic active disease) primary DLE refractory to standard of care. Subjects are assigned to the trial intervention (either daxdilimab or placebo) by randomization, which will take place, at the latest, 28 days after successful Screening. Randomization are stratified by Baseline (Day 1) Cutaneous Lupus Erythematosus Disease Area and Severity Index-Activity (CLASI-A) score (<13 vs ≥13). Approximately 99 subjects are randomized to the following 3 treatment groups in a ratio of 1:1:1 (33 subjects per group):
Group 1: Placebo
Group 2: daxdilimab 150 mg
Group 3: daxdilimab 300 mg

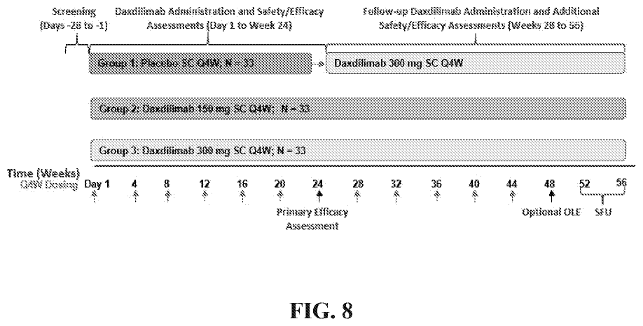

The estimated total trial duration (as shown in FIG. 8 and Tables 19A-C), from Screening to the end of the Safety Follow-up (SFU) period, are up to 60 weeks:
Screening period of approximately 4 weeks (Days −28 to −1)
Randomization (double-blinded) on Day 1
Treatment period (double-blinded) from Day 1 through Week 48
   Visit frequency: once every 4 weeks (Q4W)
   Administration of trial intervention Q4W from Day 1 through Week 44
   Efficacy and safety assessments Q4W through Week 48
   Primary assessment of the efficacy endpoints at Week 24 (after 6 administrations)
   After assessments at Week 24: Subjects in the placebo group (Group 1) are moved to a dose of 300 mg of daxdilimab for the rest of the trial. Subjects initially randomized to the daxdilimab 150 mg or 300 mg groups (Groups 2 and 3) will remain on the same dose for the rest of the trial. Although all subjects will receive active treatment from Week 24, this will not be open-label.
A SFU period of 8 weeks, from Week 48 through Week 56.
The IP are provided as a 2R vial with nominal 1 mL of 100 mg/mL daxdilimab.
The subjects are administered two 1.5 mL subcutaneous (SC) injections of the corresponding trial intervention.

All administrations of trial interventions are administered by site staff at the clinic. After the administration of trial intervention, all subjects are observed for 20 minutes, except on Day 1, Weeks 4, 24, and 28, when they are observed for at least 60 minutes.

Efficacy are assessed with:
The Cutaneous Lupus Erythematosus Disease Area and Severity Index-Activity (CLASI-A) score: A 4-point improvement in CLASI-A is considered clinically meaningful.
The Cutaneous Lupus Erythematosus Disease Area and Severity Index-Damage (CLASI-D) score: CLASI-D is not expected to improve as it represents non-reversible changes from resolved inflammation.
The Cutaneous Lupus Activity Investigator's Global Assessment (CLA-IGA) scale: The scale ranges from 0 (clear) to 4 (severe).
The Discoid Lupus Erythematosus Classification Criteria (DLECC) score (total possible score—10 points): Atrophic scarring—3 points; Location in the conchal bowl—2 points; Preference for head and neck—2 points; Dyspigmentation—1 point; Follicular hyperkeratosis/plugging—1 point; and Erythematous to violaceous color—1 point.
The Score of Activity and Damage in Discoid Lupus Erythematosus (SADDLE): Erythema, scale, induration, scarring/atrophy, and dyspigmentation are independently assessed with scores for each area in activity (0-117) and damage (0 to 78).
Digital photography to assess: i) The onset of new discoid lesions; ii) The change in characteristics (e.g., size, dyspigmentation, etc.) of the discoid lesions; and iii) Hair regrowth within the discoid lesions.

Safety are assessed by:
Measurement of physical examinations
Monitoring of vital signs
Electrocardiograms (ECGs)
Monitoring of hematology, blood chemistry, and urinalysis
Pregnancy testing
Assessment of local injection tolerability
Monitoring and recording of all adverse events (AEs)

Quality of life are assessed with:
The Patient Global Assessment (PGA) and the Patient Global Impression of Change (PGIC) scales
The Dermatology Life Quality Index (DLQI) questionnaire
The Cutaneous Lupus Erythematosus Quality of Life (CLE-QOL) questionnaire
The EQ-5D-5L questionnaire: questionnaire measuring health-related quality of life in 5 areas: mobility, self-care, usual activities, pain/discomfort, and anxiety/depression.

Blood samples are collected from all subjects to characterize the pharmacokinetics (PK), pharmacodynamics (PD), and immunogenicity of daxdilimab.
Pharmacokinetics: serum samples of approximately 3.5 mL (whole blood collection tube) are collected for measurement of serum concentrations of daxdilimab at the visits and timepoints specified in the SoA.
Pharmacodynamics: Levels of pDCs in blood (pDC flow cytometry) and tissue (e.g., immunohistochemistry [IHC] on discoid lesion biopsy [optional]), Serum IFN-α, Blood myxovirus resistance protein (MxA), and IFN gene signature levels.
Genetics: DNA are isolated from blood at Baseline to test for single nucleotide polymorphisms in genes that may be related to the development of DLE or to the mechanism of action of daxdilimab (e.g., Fc gamma receptor).
Biomarkers: i) serum samples are collected to assess changes in the levels of cytokines, chemokines, or other proteins (e.g., IFN-α, IL-17, and IFNγ) that may be related to DLE pathogenesis or the mechanism of action of daxdilimab; ii) peripheral blood mononuclear cells are collected to monitor changes in the frequency, and possibly gene expression, of immune cell populations in cryopreserved blood leukocytes; iii) whole blood samples are collected and RNA isolated to assess changes in the expression of genes or gene pathways over time that may be related to the mechanism of action of daxdilimab or DLE pathogenesis; iv) optional discoid lesion biopsies are performed to test whether daxdilimab alters levels of pDCs, inflammatory cells, and/or other biomarkers in tissue; and/or v) optional discoid lesion biopsies are performed to test whether daxdilimab alters levels of pDCs, inflammatory cells, and/or other biomarkers in tissue.
Immunogenicity: Antibodies to daxdilimab are assessed by the incidence and titer of anti-drug antibodies in serum samples collected from all subjects prior to the daxdilimab administration and according to the schedule of assessments.

### Administration

The IP daxdilimab is supplied as a 2R vial with nominal 1 mL of 100 mg/mL daxdilimab containing 20 mM L-histidine/L-histidine HCl, 240 mM sucrose, 0.02% (w/v) polysorbate 80, pH 6.0. Placebo is supplied as commercially available Normal Saline. Both trial interventions are to be administered by SC injection Q4W on Day 1 through Week 24 (Table 17). After Week 24 through Week 44, only the IP are administered to all subjects. To maintain the same number and volume of injections across all groups, the required dose are administered as 2 injections of 1.5 mL.

TABLE 17
Daxdilimab or Placebo Administration

| | | |
|---|---|---|
| Intervention Label | Investigational Product | Placebo |
| Intervention Name | daxdilimab | Placebo |
| Intervention Description | Nominal 1 mL of 100 | Normal Saline |
| | mg/mL daxdilimab | |
| | containing 20 mM L- | |
| | histidine/L-histidine HCl, | |
| | 240 mM sucrose, 0.02% | |
| | (w/v) | |
| | polysorbate 80, pH 6.0. | |
| Type | Drug | Placebo |
| Dose Formulation | 2R vial | vial |
| Unit Dose Strength(s) | 100 mg/mL daxdilimab | Normal Saline |
| Dosage Level(s) | 150 mg or 300 mg as | 2 × 1.5 mL Q4W |
| | 2 × 1.5 mL Q4W | |
| Route of Administration | SC injection | SC injection |
| Use | Experimental | Placebo |

2R = 2 mL injection vial; HCl = hydrochloric acid; Q4W = once every 4 weeks; SC = subcutaneous

### Table 18. Shows the Study Groups According to Study Design

TABLE 18
Study Groups

| | | Trial | Dose up | Dose Weeks | Intervention Form and |
|---|---|---|---|---|---|
| Group | Group type | Intervention | to Week 24 | 24 to 44 | Frequency |
| 1 | Placebo/ | Placebo/ | 0 mg (no | 300 mg | 2 × 1.5 mL SC injections |
| | Experimental | daxdilimab | drug) | | Q4W |
| 2 | Experimental | daxdilimab | 150 mg | 150 mg | 2 × 1.5 mL SC injections |
| | | | | | Q4W |
| 3 | Experimental | daxdilimab | 300 mg | 300 mg | 2 × 1.5 mL SC injections |
| | | | | | Q4W |

Q4W = once every 4 weeks; SC = subcutaneous

### Inclusion Criteria

All subjects must meet all of the following criteria to be eligible for trial participation, either at the screening and Day 1 visits, or only at one of the specified visits (screening or Day 1) as noted in the criterion:
1) Written informed consent and willing able to comply with the prescribed treatment protocol and evaluations for the duration of the trial.
2) Adult men or women ≥18 and ≤75 years of age.
3) A diagnosis of discoid lupus erythematosus for ≥6 months prior to screening supported by a history of: a) a biopsy and/or b) a clinical feature score of ≥7 on the DLECC scale if a biopsy is not available.
4) Currently active discoid lupus with all the following: a) digital photography adjudicated with central reading to confirm a currently active discoid disease lesion; and b) CLASI-A score ≥8 related to discoid lesions at Baseline.
5) Treatment refractory DLE defined as active disease despite current or historical treatment with a systemic antimalarial, methotrexate, mycophenolate, azathioprine, dapsone, corticosteroid, thalidomide, or lenalidomide, or documented history of intolerance to antimalarials and/or immunosuppressive medications.
6) Subjects with active disease who currently are on any of the following therapies must have been on a stable dosage prior to Screening and must remain on a stable dosage through Randomization and for the entire trial as described below:
   a. Antimalarials (e.g., hydroxychloroquine, chloroquine, quinacrine) must be at a stable dosage for at least 8 weeks prior to Screening and through Randomization.
   b. Methotrexate≤20 mg/week (oral or SC) at stable dosage and route of administration for at least 4 weeks prior to Screening and through Randomization.
   c. Mycophenolate mofetil≤2 g/day or mycophenolic acid≤1.44 g/day at stable dosage for at least 4 weeks prior to Screening and through Randomization.
   d. Azathioprine must be stable for at least 4 weeks prior to Screening and through Randomization.
   e. Corticosteroid equivalent to prednisone≤10 mg/day at stable dosage for at least 4 weeks prior to Screening and through Randomization.
   f. Topical corticosteroids and calcineurin inhibitors at stable dosage for at least 1 week prior to Screening and through Randomization.
7) Women of childbearing potential (WOCBP) must have a negative serum pregnancy test at screening and a negative urine pregnancy test at Day 1. Women must agree to use 2 approved forms of contraception, one of which is recommended to be hormonal contraceptives, from at least 4 weeks prior to Day 1 until at least 6 months (approximately 5 half-lives) after the last IP administration or the end of the trial, whichever is longer and should refrain from egg retrieval and egg donation during the trial and until at least 6 months (approximately 5 half-lives) after the last IP administration.
8) Men must agree to use one of the highly effective contraceptive methods from Day 1 until at least 3 months (approximately 5 half-lives) after the last IP administration and refrain from donating sperm during this period.

### Exclusion Criteria

### General Exclusion Criteria:

1. Individuals involved in the conduct of the trial, their employees, or immediate family members of such individuals.
2. Participation in another clinical trial with an investigational drug within 4 weeks prior to Randomization or within 5 published half-lives, whichever is longer.

### Medical Conditions:

3. Any condition that, in the opinion of the Investigator, would interfere with evaluation of the IP or interpretation of subject safety or trial results.
4. Weight ≥160 kg (352 pounds) at Screening.
5. History of allergy, hypersensitivity reaction, or anaphylaxis to any component of the IP or to a previous monoclonal antibody (mAb) or human immunoglobulin (Ig) therapy.
6. Breastfeeding or pregnant women or had spontaneous or induced abortion, still or live birth, or pregnancy ≤4 weeks prior to Screening through Randomization.
7. History of drug or alcohol abuse
8. Major surgery within 8 weeks prior to Screening or elective surgery planned from Screening through Week 48.
9. Splenectomy
10. History of clinically significant cardiac disease including unstable angina, myocardial infarction, congestive heart failure within 6 months prior to Randomization; arrhythmia requiring active therapy, except for clinically insignificant extra systoles, or minor conduction abnormalities; or presence of clinically significant abnormality on ECG
11. History of cancer within the past 5 years, except as follows: i) in situ carcinoma of the cervix treated >12 months prior to Screening; or ii) Cutaneous basal cell or squamous cell carcinoma treated with curative therapy.
12. Any underlying condition predisposes the subject to infection
13. Subject who has given >50 mL of blood or plasma within 30 days of Screening or >499 mL of blood or plasma within 56 days of Screening (during a clinical trial or at a blood bank donation) or plans to give blood or plasma during their participation in the trial or up to 6 months after the last IP administration, whichever is longer.
14. Transfusion with blood, packed red blood cells, platelets or treatment with plasmapheresis, or plasma exchange within 8 weeks prior to Randomization and for the total duration of the trial participation.

### Laboratory Criteria:

15. Known history of a primary immunodeficiency such as human immunodeficiency virus (HIV) infection, or a positive result for HIV infection.
16. At Screening, any of the following per central laboratory tests:
   a. Aspartate aminotransferase (AST)>2.5×upper limit of normal (ULN)
   b. Alanine aminotransferase (ALT)>2.5×ULN
   c. Total bilirubin (TBL)>1.5×ULN (unless due to Gilbert's syndrome)
   d. Neutrophil count<1500/μL (or <1.5×10⁹/L)
   e. Platelet count<135,000/μL (or <135×10⁹/L)
   f. Hemoglobin<10 g/dL (or <100 g/L)
   g. Total lymphocyte count<800/μL (or <0.8×10⁹/L)
   h. Antinuclear antibody titer ≥1:320
17. Confirmed positive test for hepatitis B virus serology defined as: i) Hepatitis B surface antigen, or ii) Hepatitis B core antibody
18. Positive test for hepatitis C virus antibody unless documented as having had successful treatment of active hepatitis C infection.
19. Active tuberculosis (TB), or a positive IFNγ release assay (IGRA) test at Screening.
20. Any severe herpes virus family infection, such as, but not limited to, disseminated herpes, herpes encephalitis, recent recurrent herpes zoster (defined as 2 episodes within the last 2 years), or ophthalmic herpes.
21. Any herpes zoster, CMV, or Epstein-Barr virus infection
22. Any of the following within 30 days prior to signing the ICF and through Randomization
   a. Clinically significant active infection in the opinion of the Investigator, including ongoing, and chronic infection requiring antibiotics or antiviral medication.
   b. Any infection requiring hospitalization or treatment with intravenous anti-infectives.
   c. A participant with a documented positive severe acute respiratory syndrome-coronavirus-2 (SARS-COV-2) test may be rescreened at least 2 weeks after a positive test if the participant is asymptomatic and at least 3 weeks after symptomatic coronavirus disease 2019 (COVID-19) illness.
23. Opportunistic infection requiring hospitalization or parenteral antimicrobial treatment within 2 years prior to Randomization.
24. Any acute illness or evidence of clinically significant active infection on Day 1.
25. Participants who have COVID-19 or other significant infection, or in the judgment of the Investigator, may be at a high risk of COVID-19 or its complications should not be randomized.

### Disease-Related Criteria:

26. Systemic lupus erythematosus.
27. Current diagnosis of a systemic connective tissue disease.
28. Current inflammatory skin disease other than DLE

### Prior/Concomitant Therapy

29. Use of systemic (≥10 mg/day of prednisone equivalent) or intralesional steroids, or nonbiologic immunomodulatory or immunosuppressive agents (e.g., dapsone, danazol, calcineurin inhibitors, sulfasalazine, mizoribine, mechanistic target of rapamycin inhibitors, retinoids, thalidomide, lenalidomide, adrenocorticotropic hormone analogs, dehydroepiandrosterone), or Janus kinase inhibitors within 4 weeks prior Randomization
30. Previous treatment with any biologic B-cell-depleting therapy (e.g., rituximab, ocrelizumab, or ofatumumab) within 12 months or other B-cell targeting therapy (e.g., belimumab) within 3 months
31. Received previous treatment with pDC inhibiting therapies (e.g., daxdilimab, anti-BDCA2 [BIIB059].
32. Any biologic or antiproliferative agents within 4 weeks
33. Received experimental drug either 30 days, 5 half-lives of the agent, or twice the duration of the biological effect of the agent, whichever is longer
34. Received a live-attenuated vaccine within 4 weeks.

### Permitted Therapy

The following therapies are permitted: Antimalarials (e.g., hydroxychloroquine, chloroquine, quinacrine) must be at a stable dosage for at least 8 weeks prior to Screening and through Randomization.
Methotrexate ≤20 mg/week (oral or SC) at stable dosage and route of administration for at least 4 weeks prior to Screening and through Randomization.
Mycophenolate mofetil ≤2 g/day or mycophenolic acid ≤1.44 g/day at stable dosage for at least 4 weeks prior to Screening and through Randomization.
Azathioprine must be stable for at least 4 weeks prior to Screening and through Randomization.
Corticosteroids equivalent to prednisone ≤10 mg/day at stable dose for at least 4 weeks prior to Screening and through Randomization. Investigators may taper prednisone as clinically indicated.
Topical corticosteroids and calcineurin inhibitors at stable dosage for at least 1 week prior to Screening and through Randomization.
Non-immunomodulatory medication used for routing treatment of other medical conditions such as hypertension or diabetes.

### Prohibited Therapy

Table 19 lists prohibited medications that are not to be used from the defined washout periods before the first administration of trial intervention at the Day 1 visit through the last trial visit.

TABLE 19
Prohibited Therapies and Procedures

| | Washout period prior to |
|---|---|
| Prohibited medication, products, and procedures | first dose (Day 1) |
| Systemic (>10 mg/day prednisone equivalent) or intralesional | 4 weeks |
| steroids, or nonbiologic immunomodulatory or immunosuppressive | |
| agents (eg, dapsone, danazol, calcineurin inhibitors, sulfasalazine, | |
| mizoribine, mechanistic target of rapamycin inhibitors, retinoids, | |
| thalidomide, lenalidomide, adrenocorticotropic hormone analogs, | |
| dehydroepiandrosterone), or JAK inhibitors. | |
| B-cell targeting therapy (eg, belimumab) | 3 months |
| B-cell-depleting therapy (eg, rituximab, ocrelizumab, or ofatumumab) | 12 months |
| Any pDC inhibiting therapies (eg, daxdilimab, anti-BDCA2 | Lifetime |
| [BIIB059]). | |
| Any biologic or antiproliferative agents | 4 weeks or 5 half-lives, |
| | whichever is longer |
| Experimental drugs | 30 days, 5 half-lives, or |
| | twice the duration of the |
| | biological effect of the |
| | agent, whichever is |
| | longer. |
| Live-attenuated vaccine | 4 weeks |

BDCA2 = blood dendritic cell antigen 2; JAK, Janus kinase; pDC = plasmacytoid dendritic cell

### Discontinuation of Study Intervention

An individual subject will not receive any further administration if any of the following occur in the subject:
A CTCAE (Common Terminology for Adverse Events) Grade 3 or higher allergic reaction to the trial intervention.
A CTCAE Grade 3 or higher infection considered related to the trial intervention.
Other AE that contraindicates further dosing in the opinion of the Investigator and/or the Sponsor, or medical monitor.
Withdrawal of consent from further treatment with trial intervention.
Receipt of prohibited therapy (see Section 6.9.2).
Subject is determined to have met one or more of the exclusion criteria or failed to meet all the inclusion criteria, such as systemic lupus or lupus nephritis requiring more aggressive treatment, for trial participation and there is a potential safety risk associated with continuation identified upon consultation with the medical monitor.
Pregnancy or a decision to become pregnant.
Liver function abnormalities:
   ALT or AST≥8×ULN
   ALT or AST≥5×ULN for more than 2 weeks
   ALT or AST≥3×ULN with the appearance of fatigue, nausea, vomiting, right upper quadrant pain or tenderness, fever, rash, and/or eosinophilia (≥5%).
Clinically significant changes from baseline in corrected QT interval using Bazett's [QTcB] or Fridericia's [QTcF] formulas.

### INCORPORATION BY REFERENCE

This patent application incorporates by reference in their entireties for all purposes the following patent publication: WO2021113702. All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is from about 100-350 mg.

2. The method of claim 1, wherein the autoimmune disease is selected from the group consisting of discoid lupus erythematosus (DLE), systemic lupus erythematosus (SLE), lupus nephritis, Dermatomyositis, Anti-Synthetase Inflammatory Myositis, and alopecia areata.

3. (canceled)

4. The method of claim 1, wherein the administering is effective in reducing:
a) a level of plasmacytoid dendritic cells (pDCs) in a tissue of the subject;
b) a type I interferon gene signature (IFNGS); or
c) the level of plasmacytoid dendritic cells (pDCs) in the tissue of the subject and the type I IFNGS, each of each is as compared to a baseline level of the subject before the administering.

5-12. (canceled)

13. The method of claim 1, wherein the ILT7-binding protein is administered with one or more additional therapies.

14. The method of claim 13, wherein the one or more additional therapies comprises a corticosteroid.

15. The method of claim 14, wherein the corticosteroid is prednisone.

16. The method of claim 13, wherein the administration of the one or more additional therapies is tapered.

17. The method of claim 1, wherein the pharmaceutically effective amount of the ILT7-binding protein is from about 150-300 mg.

18. The method of claim 1, wherein the pharmaceutically effective amount of the ILT7-binding protein is from about 200-300 mg.

19. The method of claim 1, wherein the ILT7-binding protein is administered once about every four weeks or once about every twelve weeks.

20. The method of claim 1, wherein the pharmaceutically effective amount of the ILT7-binding protein is about 100 mg, 150 mg, 200 mg, or 300 mg.

21. A method of treating discoid lupus erythematosus (DLE) in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 100 mg to about 300 mg.

22. A method of treating systemic lupus erythematosus (SLE) in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 100 mg to about 300 mg.

23. A method of treating lupus nephritis in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 100 mg to about 300 mg.

24. A method of treating alopecia areata in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 100 mg to about 300 mg.

25. A method of treating dermatomyositis in a subject in need thereof, the method comprising administering to the subject a pharmaceutically effective amount of an immunoglobulin-like transcript 7 (ILT7)-binding protein.

26. A method of treating Anti-Synthetase Inflammatory Myositis in a subject in need thereof, the method comprising administering to the subject a pharmaceutically effective amount of an immunoglobulin-like transcript 7 (ILT7)-binding protein.

27. The method of claim 25, wherein the pharmaceutically effective amount of the ILT7-binding protein is from about 100 mg to about 300 mg.

28. The method of claim 1, wherein the ILT7 binding protein is an antibody that comprises heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOs: 3, 4, 5, 6, 7, and 8, respectively.

29. The method of claim 28, wherein the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2.

30. The method of claim 28, wherein the ILT7-binding protein is an antibody comprising a heavy chain variable region (VH) of SEQ ID NO: 1 and a light chain variable region (VL) of SEQ ID NO:2.

31. The method of claim 1, wherein the ILT7 binding protein is afucosylated.

32. The method of claim 1, wherein the ILT7 binding protein comprises a heavy chain comprising SEQ ID NO: 10; and a light chain comprising SEQ ID NO: 9.

33. The method of claim 1, wherein the administration is subcutaneous.

34. The method of claim 1, wherein the subject is administered the ILT7-binding protein every 4 weeks.

35. The method of claim 1, wherein the subject is administered the ILT7-binding protein every 12 weeks.

36. The method of claim 1, wherein prior to the administering, the subject is administered at least one initial dose of the ILT7-binding protein.

37. The method of claim 36, wherein the at least one initial dose is administered every 2 weeks for 1, 2, 3, 4, 5, or more doses.

38. The method of claim 36, wherein the at least one initial dose is about 100-300 mg.

39. A method of treating lupus nephritis in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 100 mg every 2 weeks for up to 4 weeks followed by 100 mg every 4 weeks thereafter.

40-41. (canceled)

42. A method of treating lupus nephritis in a subject in need thereof, the method comprising administering to the subject a pharmaceutically effective amount of immunoglobulin-like transcript 7 (ILT7)-binding protein, wherein the pharmaceutically effective amount of the ILT7-binding protein is about 300 mg every 2 weeks for up to 4 weeks followed by 300 mg every 12 weeks thereafter.

43. The method of claim 19, comprising a first dose, a second dose two weeks after the first dose, and subsequent doses every 4 weeks following the first initial dose.

44. The method of claim 19, comprising a first dose, a second dose four weeks after the first dose, and subsequent doses every 12 weeks following the first initial dose.

45. The method of claim 26, wherein the pharmaceutically effective amount of the ILT7-binding protein is from about 100 mg to about 300 mg.


SEQUENCE LISTING (10 sequences)

SEQ ID NO: 1 - protein, 122 aa, Artificial Sequence
  Synthetic polypeptide
QVQLQQSGAEVKKPGASVKVSCKASGYTFTSYGISWVRQAPGQGLEWMGWISAYNGNTNYAQKLQGRVTMTTDTSTSTAYMELRSLRSDDTAVYYCARNGLWGWDSDAFDIWGRGTLVTVSS

SEQ ID NO: 2 - protein, 110 aa, Artificial Sequence
  Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTVVFGGGTKVTVL

SEQ ID NO: 3 - protein, 5 aa, Artificial Sequence
  Synthetic peptide
SYGIS

SEQ ID NO: 4 - protein, 17 aa, Artificial Sequence
  Synthetic peptide
WISAYNGNTNYAQKLQG

SEQ ID NO: 5 - protein, 13 aa, Artificial Sequence
  Synthetic peptide
NGLWGWDSDAFDI

SEQ ID NO: 6 - protein, 14 aa, Artificial Sequence
  Synthetic peptide
TGTSSDVGGYNYVS

SEQ ID NO: 7 - protein, 7 aa, Artificial Sequence
  Synthetic peptide
DVSNRPS

SEQ ID NO: 8 - protein, 10 aa, Artificial Sequence
  Synthetic peptide
SSYTSSSTVV

SEQ ID NO: 9 - protein, 452 aa, Artificial Sequence
  Synthetic polypeptide
QVQLQQSGAEVKKPGASVKVSCKASGYTFTSYGISWVRQAPGQGLEWMGWISAYNGNTNYAQKLQGRVTMTTDTSTSTAYMELRSLRSDDTAVYYCARNGLWGWDSDAFDIWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 216 aa, Artificial Sequence
  Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTVVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS